(19) United States
(12) Patent Application Publication
(10) Pub. No.: US 2025/0120997 A1
(43) Pub. Date: Apr. 17, 2025

(54) INTRASPINAL DELIVERY OF THERAPEUTIC AGENTS

(71) Applicants: The Trustees of the University of Pennsylvania, Philadelphia, PA (US); University of Szeged, Szeged (HU)

(72) Inventors: Norbert Pardi, Philadelphia, PA (US); Drew Weissman, Philadephia, PA (US); László Gal, Szeged (HU); Krisztián Pajer, Szeged (HU); Antal Nogradi, Szeged (HU)

(21) Appl. No.: 18/708,792

(22) PCT Filed: Nov. 9, 2022
(86) PCT No.: PCT/US2022/079557

§ 371 Date: May 9, 2024

Related U.S. Application Data
(60) Provisional application No. 63/277,415, filed on Nov. 9, 2021.

(51) Int. Cl. A61K 31/712; A61K 9/00; A61K 9/127; A61K 48/00; A61P 25/02

(52) U.S. Cl. CPC A61K 31/712; A61K 9/0085; A61K 9/127; A61K 48/0008; A61K 48/0058; A61P 25/02

(57) ABSTRACT

Disclosed herein is a composition including a nucleoside-modified mRNA encapsulated in a lipid nanoparticle wherein the nucleoside-modified mRNA encodes IL10, IL-6, MIP1a, GDNF (glial cell line-derived neurotrophic factor), or a combination thereof. A method of treated spinal cord injury is also described.

### CROSS-REFERENCE TO RELATED APPLICATIONS

This application claims benefit to U.S. Provisional Application No. 63/277,415, filed Nov. 9, 2021, and the entirety of its contents are herein incorporated by reference.

### STATEMENT REGARDING FEDERALLY SPONSORED RESEARCH OR DEVELOPMENT

This invention was made with government support under AI146101 awarded by the National Institutes of Health. The government has certain rights in the invention.

### BACKGROUND OF THE INVENTION

Traumatic spinal cord injuries (SCIs) represent a heterogenous and complex pathophysiology that affects 1 million North Americans, a disproportionate number of whom are younger than 30 years (Ahuja et al., F1000Research, 2016 May 27; F1000 Faculty Review 5:1017). Injury of the spinal cord results in loss of both grey and white matter, i.e., produces a segmental spinal cord lesion as well as disconnects the brain and rostral spinal cord from the caudal parts of the cord (Ulndreaj et al., F1000Research, 2017 Oct. 30; 6:1907). SCIs in humans and other mammals are not followed by the replacement of lost neurons or regrowth of injured axons, but they lead to permanent, incurable functional deficits. Many of these injuries affect the long ascending and descending tracts thus separating the lower spinal cord segments from the higher motor and sensory centres (Alizadeh et al., Frontiers in Neurology, 2019 Mar. 22; 10:282). The primary physical injury is followed by a cascade of events, called secondary injury, the extent of which is always larger than that of the primary injury (Ulndreaj et al., F1000Research, 2017 Oct. 30; 6:1907). This phase is associated with a second set of symptoms including inflammation, neuronal and glial apoptosis, glutamate excitotoxicity, potential disruption of the blood-brain barrier, axonal demyelination, and reactive astrogliosis, leading to the formation of a large cavity (Alizadeh et al., Frontiers in Neurology, 2019 Mar. 22; 10:282). Together with scar formation, axonal demyelination and Wallerian degeneration give rise to axonal retraction (Alizadeh et al., Frontiers in Neurology, 2019 Mar. 22; 10:282, Ulndreaj et al. F1000Research, 2017 Oct. 30; 6:1907).

Administration of interleukin-10 (IL-10) protein has shown promise in the treatment/cure of SCI but safe and efficient drug delivery to the injured spinal cord represents an elusive goal (Kabu et al., Journal of Controlled Release, 2015 Dec. 10; 219:141-154, Hoshino et al., Scientific Reports, 2019 Jul. 8; 9(1):9844, Margul et al., Bioengineering and Translational Medicine, 2016 June; 1(2):136-148, Park et al., Molecular Therapy, 2018 July 5; 26(7):1756-1770). Several studies have demonstrated that administration of IL-10 protein following experimental spinal cord injury induced neuronal survival and a certain level of functional recovery (Jackson et al., Virology, 2005 June 5; 336(2):173-83, Thompson et al., Journal of Neurotrauma, 2013 Aug. 1; 30(15):1311-24, Pajer et al., Experimental Neurology Journal, 2014 November; 261:180-9, Pajer et al., Experimental Neurology Journal, 2015 July; 269:188-201, Park et al., Molecular Therapy, 2018 Jul. 5; 26(7):1756-1770, Pajer et al., Journal of Neurotrauma, 2019 Nov. 1; 36(21):2977-2990, Pajer et al., Neural Regeneration Research, 2020 October; 15(10):1844-1845, Smith et al., Tissue Engineering Part A, 2020 June; 26(11-12):672-682).

The short half-life of IL-10 and its instability in circulation represents a major challenge, necessitating repeated administration (Margul et al. Bioengineering and Translational Medicine, 2016 June; 1(2):136-148). Several strategies have been developed for the safe and efficient delivery of neurotrophic factors or cytokines to injured spinal cord but these approaches are often invasive and associated with adverse effects (Blits et al., Experimental Neurology Journal, 1999 November; 160(1):256-67, Kumar et al., Current Pharmaceutical Design, 2015; 21(12):1517-28). Therefore, development of a safe and well-controlled tool for cytokine delivery where the combined action of the identified biomolecules points towards definite morphological and functional improvement is critically important.

Thus there is a need in the art for improved compositions and methods for the delivery of therapeutic agents for the treatment of spinal cord injuries.

### SUMMARY OF THE INVENTION

In one aspect, the present invention relates to compositions for modulating a microenvironment of an injured spinal cord in a subject. In one embodiment, the composition comprises a nucleoside-modified mRNA encapsulated in a lipid nanoparticle, wherein the nucleoside-modified mRNA encodes IL10, IL-6, MIP1a, GDNF (glial cell line-derived neurotrophic factor), or a combination thereof.

In one embodiment, the composition comprises a nucleoside-modified mRNA sequence encoding IL10 and a nucleoside-modified mRNA sequence encoding IL-6. In one embodiment, the composition comprises a nucleoside-modified mRNA sequence encoding IL10 and a nucleoside-modified mRNA sequence encoding MIP1a. In one embodiment, the composition comprises a nucleoside-modified mRNA sequence encoding IL10 and a nucleoside-modified mRNA sequence encoding GDNF. In one embodiment, the composition comprises a nucleoside-modified mRNA sequence encoding IL10, a nucleoside-modified mRNA sequence encoding IL-6, a nucleoside-modified mRNA sequence encoding MIP1a, and a nucleoside-modified mRNA sequence encoding GDNF. In one embodiment, the nucleoside-modified mRNA comprises m1Ψ.

In one embodiment, the nucleoside-modified mRNA encodes human IL10, wherein the nucleoside-modified mRNA is encoded by an optimized DNA sequence comprising SEQ ID NO: 5. In one embodiment, the nucleoside-modified mRNA encodes for human IL10, comprising the amino acid sequence of SEQ ID NO:1.

In one embodiment, the nucleoside-modified mRNA encodes for human IL6, wherein the nucleoside-modified mRNA is encoded by an optimized DNA sequence comprising SEQ ID NO: 6. In one embodiment, the nucleoside-modified mRNA encodes for human IL6, comprising the amino acid sequence of SEQ ID NO:2.

In one embodiment, the nucleoside-modified mRNA encodes for human MIP1a, wherein the nucleoside-modified mRNA is encoded by an optimized DNA sequence comprising SEQ ID NO: 7. In one embodiment, the nucleoside-modified mRNA encodes for human MIP1a, comprising the amino acid sequence of SEQ ID NO:3.

In one embodiment, the nucleoside-modified mRNA encodes for human GDNF, wherein the nucleoside-modified mRNA is encoded by an optimized DNA sequence comprising SEQ ID NO: 8. In one embodiment, the nucleoside-modified mRNA encodes for human GDNF, comprising the amino acid sequence of SEQ ID NO:4.

In one embodiment, the composition comprises a nucleoside-modified mRNA sequence encoding human IL10, wherein the nucleoside-modified mRNA is encoded by the optimized DNA sequence comprising SEQ ID NO: 5, a nucleoside-modified mRNA sequence encoding human IL-6, wherein the nucleoside-modified mRNA is encoded by the optimized DNA sequence comprising SEQ ID NO: 6, a nucleoside-modified mRNA sequence encoding human MIP1a, wherein the nucleoside-modified mRNA is encoded by the optimized DNA sequence comprising SEQ ID NO: 7, and a nucleoside-modified mRNA sequence encoding human GDNF, wherein the nucleoside-modified mRNA is encoded by the optimized DNA sequence comprising SEQ ID NO: 8.

In one embodiment, the composition comprises a nucleoside-modified mRNA sequence encoding human IL10 comprising the amino acid sequence of SEQ ID NO:1, a nucleoside-modified mRNA sequence encoding human IL-6 comprising the amino acid sequence of SEQ ID NO:2, a nucleoside-modified mRNA sequence encoding human MIP1a comprising the amino acid sequence of SEQ ID NO:3, and a nucleoside-modified mRNA sequence encoding human GDNF comprising the amino acid sequence of SEQ ID NO:4.

In one aspect, the present invention relates to methods of treating a spinal cord injury. In one embodiment, the method comprises intra spinally applying a composition comprising a nucleoside-modified mRNA encapsulated in a lipid nanoparticle, wherein the nucleoside-modified mRNA encodes IL10, IL-6, MIP1a, GDNF (glial cell line-derived neurotrophic factor), or a combination thereof. In one embodiment, the composition comprises a nucleoside-modified mRNA sequence encoding IL10 and a nucleoside-modified mRNA sequence encoding IL-6. In one embodiment, the composition comprises a nucleoside-modified mRNA sequence encoding IL10 and a nucleoside-modified mRNA sequence encoding MIP1a. In one embodiment, the composition comprises a nucleoside-modified mRNA sequence encoding IL10 and a nucleoside-modified mRNA sequence encoding GDNF. In one embodiment, the composition comprises a nucleoside-modified mRNA sequence encoding IL10, a nucleoside-modified mRNA sequence encoding IL-6, a nucleoside-modified mRNA sequence encoding MIP1a, and a nucleoside-modified mRNA sequence encoding GDNF. In one embodiment, the nucleoside-modified mRNA comprises m1Ψ.

In one embodiment, the method comprises intra spinally applying a composition comprising a nucleoside-modified mRNA encoding IL10 encapsulated in a lipid nanoparticle. In one embodiment, the nucleoside-modified mRNA encodes human IL10, wherein the nucleoside-modified mRNA is encoded by an optimized DNA sequence comprising SEQ ID NO: 5. In one embodiment, the nucleoside-modified mRNA encodes for human IL10, comprising the amino acid sequence of SEQ ID NO:1.

In one embodiment, the method comprises intra spinally applying a composition comprising a nucleoside-modified mRNA encoding IL6 encapsulated in a lipid nanoparticle. In one embodiment, the nucleoside-modified mRNA encodes for human IL6, wherein the nucleoside-modified mRNA is encoded by an optimized DNA sequence comprising SEQ ID NO: 6. In one embodiment, the nucleoside-modified mRNA encodes for human IL6, comprising the amino acid sequence of SEQ ID NO:2.

In one embodiment, the method comprises intra spinally applying a composition comprising a nucleoside-modified mRNA encoding MIP1a encapsulated in a lipid nanoparticle. In one embodiment, the nucleoside-modified mRNA encodes for human MIP1a, wherein the nucleoside-modified mRNA is encoded by an optimized DNA sequence comprising SEQ ID NO: 7. In one embodiment, the nucleoside-modified mRNA encodes for human MIP1a, comprising the amino acid sequence of SEQ ID NO:3.

In one embodiment, the method comprises intra spinally applying a composition comprising a nucleoside-modified mRNA encoding GDNF encapsulated in a lipid nanoparticle. In one embodiment, the nucleoside-modified mRNA encodes for human GDNF, wherein the nucleoside-modified mRNA is encoded by an optimized DNA sequence comprising SEQ ID NO: 8. In one embodiment, the nucleoside-modified mRNA encodes for human GDNF, comprising the amino acid sequence of SEQ ID NO:4.

In one embodiment, the method comprises intra spinally applying a composition comprising a nucleoside-modified mRNA encoding IL10, a nucleoside-modified mRNA encoding IL-6, a nucleoside-modified mRNA encoding MIP1a, and a nucleoside-modified mRNA encoding GDNF encapsulated in a lipid nanoparticle. In one embodiment, the composition comprises a nucleoside-modified mRNA sequence encoding human IL10, wherein the nucleoside-modified mRNA is encoded by the optimized DNA sequence comprising SEQ ID NO: 5, a nucleoside-modified mRNA sequence encoding human IL-6, wherein the nucleoside-modified mRNA is encoded by the optimized DNA sequence comprising SEQ ID NO: 6, a nucleoside-modified mRNA sequence encoding human MIP1a, wherein the nucleoside-modified mRNA is encoded by the optimized DNA sequence comprising SEQ ID NO: 7, and a nucleoside-modified mRNA sequence encoding human GDNF, wherein the nucleoside-modified mRNA is encoded by the optimized DNA sequence comprising SEQ ID NO: 8.

In one embodiment, the method comprises intra spinally applying a composition comprising a nucleoside-modified mRNA encoding IL10, a nucleoside-modified mRNA encoding IL-6, a nucleoside-modified mRNA encoding MIP1a, and a nucleoside-modified mRNA encoding GDNF encapsulated in a lipid nanoparticle. In one embodiment, the composition comprises a nucleoside-modified mRNA sequence encoding human IL10 comprising the amino acid sequence of SEQ ID NO:1, a nucleoside-modified mRNA sequence encoding human IL-6 comprising the amino acid sequence of SEQ ID NO:2, a nucleoside-modified mRNA sequence encoding human MIP1a comprising the amino acid sequence of SEQ ID NO:3, and a nucleoside-modified mRNA sequence encoding human GDNF comprising the amino acid sequence of SEQ ID NO:4.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1F, depicts the results of representative experiments showing eGFP expression in intact rat spinal cord following intraspinal delivery of mRNA-LNPs encoding eGFP. FIG. 1A depicts a representative image demonstrating a lack of eGFP expression in intact rat spinal cord without intraspinal administration of mRNA-LNP encoding eGFP. FIG. 1B depicts a representative image depicting eGFP expression in parasagittal section of rat spinal cord 1, 5 and 21 days after the intraspinal application of mRNA-LNPs. FIG. 1C depicts representative quantification of the fluorescence signal of eGFP in rat spinal cord injected with 3.0 μg mRNA-LNPs. Data presented as mean±s.e.m.; n=4. FIG. 1D depicts representative images of spinal cord cross-sections demonstrating immunohistochemical detection of eGFP in astrocytes 1 day after intraspinal mRNA-LNP administration. FIG. 1E depicts representative images of spinal cord cross-sections demonstrating immunohistochemical detection of eGFP in microglia/macrophages 1 day after intraspinal mRNA-LNP administration. FIG. 1F depicts representative images of spinal cord cross-sections demonstrating immunohistochemical detection of eGFP in neurons 1 day after intraspinal mRNA-LNP administration. In FIGS. 1D-1F, arrowheads show the co-localized cells. For FIGS. 1A and 1B, scale bar=1 mm. For FIGS. 1D-1F, scale bar=200 μm.

FIG. 2, comprising FIG. 2A through FIG. 2L, depicts representative eGFP expression in injured rat spinal cord following intraspinal delivery of mRNA-LNPs encoding eGFP. FIG. 2A depicts representative images demonstrating a lack of eGFP expression in SCI group. FIG. 2B depicts representative images of eGFP expression in mRNA-GFP group up to 21 days after intraspinal mRNA-LNP administration. FIG. 2C depicts representative quantification of eGFP expression in the parasagittal sections of spinal cord in various time points in mRNA-GFP group. Data presented as mean±s.e.m.; n=4. FIG. 2D depicts representative imaging of astrocytes expressing eGFP 1 day after intraspinal delivery of mRNA-LNP encoding eGFP. FIG. 2E depicts higher magnification of the indicated region of FIG. 2D. FIG. 2F depicts further magnification of FIG. 2E. FIG. 2G depicts representative imaging of neurons expressing eGFP 1 day after intraspinal delivery of mRNA-LNP encoding eGFP. FIG. 2H depicts higher magnification of the indicated region of FIG. 2G. FIG. 2I depicts further magnification of FIG. 2H. FIG. 2J depicts representative imaging of microglia/macrophages expressing eGFP 1 day after intraspinal delivery of mRNA-LNP encoding eGFP. FIG. 2K depicts higher magnification of the indicated region of FIG. 2J. FIG. 2L depicts further magnification of FIG. 2J. The arrows of FIGS. 2E-2F, 2H-2I, and 2K-2J show the presence of GFAP, TUBB3, and GSA-B4-positive cells co-localizing with GFP. For FIGS. 2A and 2B, scale bar=1 mm. For FIGS. 2D, 2G and 2J, scale bar=500 μm. For FIGS. 2E, 2H, and 2K, scale bar=100 μm. For FIGS. 2F, 2I, and 2L, scale bar=50 μm.

FIG. 3, comprising FIG. 3A through FIG. 3M, depicts representative hIL-10 expression in injured spinal cord following intraspinal delivery of mRNA-LNPs encoding hIL-10. FIG. 3A depict representative images demonstrating a lack of hIL-10 expression detected in injured rat spinal cord (SCI group). FIG. 3B depicts representative images demonstrating hIL-10 expression in parasagittal section of rat spinal cord 1, 2 and 5 days after intraspinal delivery of mRNA-LNP encoding hIL-10. FIG. 3C depicts representative quantification of ELISA-evaluated production of hIL-10 in injured spinal cords of control animals (SCI) and animals injected with mRNA-LNP encoding hIL-10 (mRNA-hIL-10). FIG. 3D depicts representative quantification of ELISA-evaluated production of hIL-10 in serum of control animals and animals injected with mRNA-LNP encoding hIL-10. FIG. 3E depicts representative imaging of rat neurons expressing hIL-10 1 day after intraspinal mRNA-LNP administration. FIG. 3F depicts a magnified region of FIG. 3F. FIG. 3G depicts a magnified region of FIG. 3G. FIG. 3H depicts representative imaging of rat astrocytes around the lesion area expressing hIL-10 1 day after intraspinal mRNA-LNP administration. FIG. 3I depicts a magnified region of FIG. 3H. FIG. 3J depicts a magnified region of FIG. 3I. FIG. 3K depicts representative imaging of co-localization of GSA-B4 positive cells with hIL-10 1 day after intraspinal mRNA-LNP administration in the lesion area. FIG. 3L depicts a magnified region of FIG. 3K. FIG. 3M depicts a magnified region of FIG. 3K. For FIGS. 3C and 3D, data presented as mean±s.e.m.; n=4. For FIGS. 3G, 3J, and 3M, arrows show the co-localized cells. For FIGS. 3A and 3B, scale bar=800 μm. For FIGS. 3E, 3H, and 3K, scale bar=750 μm. For FIGS. 3F and 3L, scale bar=30 μm. For FIG. 3I, scale bar=25 μm. For FIGS. 3G, 3J, and 3M, scale bar=20 μm.

FIG. 4, comprising FIG. 4A through FIG. 4E, depicts representative improvement in locomotor function after delayed intraspinal administration of mRNA-LNP encoding hIL-10. FIG. 4A depicts representative results of open field locomotor testing (BBB), demonstrating significant improvement of hIL-10 treated animals (mRNA-hIL-10 and hIL-10 osmotic pump group) compared with their controls. Data analyzed using a two-way ANOVA; *, p<0.05 between hIL-10 treated group (mRNA-hIL-10 and hIL-10 osmotic pump group) group and SCI/mRNA-GFP; n=8. FIG. 4B depicts a representative image showing every position of the measured bones during one intact step cycles from the lateral aspect. The step cycle can be divided into stance phase (black) and swing phase (red). FIG. 4C depicts representative measurement of the rear-view parameters based on the angle enclosed by a selected bone and the floor plate. The intact value is displayed in blue, while green and red angles are representing the deviations followed by contusion injury, respectively. White arrows show the deviation in both directions. FIG. 4D depicts representative quantification and images of kinematic analysis of animals, from the side, of the various groups 9 weeks after injury. FIG. 4E depicts representative kinematic analysis and images, from the rear, of the animals in the various groups 9 weeks after injury. Significant improvement is observed in the parameters of the hIL-10 treated animals (mRNA-hIL-10 and hIL-10 osmotic pump group) compared with SCI and mRNA-GFP groups. For FIGS. 4D and 4E, data analyzed using one-way ANOVA with LSD multiple comparisons test; data presented as mean s.e.m.; n=8; * p<0.05 between SCI/mRNA-GFP vs. mRNA-hIL-10/hIL-10 osmotic pump group; #, p<0.05 between SCI/mRNA-GFP vs. mRNA-IL10 group.

FIG. 5, comprising FIG. 5A through FIG. 5H, depicts representative promoted tissue sparing by delayed intraspinal administration of mRNA-LNPs encoding hIL-10. FIG. 5A depicts representative images of cresyl-violet stained sections taken at 100 μm rostrally from the lesion epicentre. Scale bar=500 μm. FIG. 5B depicts representative quantification of lesion area, demonstrating that hIL-10 treatment resulted in significantly reduced size of injury following SCI. FIG. 5C depicts representative quantification of improved tissue sparing rostral and caudal to lesion epicentre in hIL-10 treated groups (mRNA-hIL-10 and hIL-10 osmotic pump) compared with SCI and mRNA-GFP animals. FIG. 5D depicts a schematic image demonstrating the method of retrograde labelling. Fast Blue crystal was placed into the L3 spinal segment after the right hemisection gap. FIG. 5E depicts representative imaging of retrogradely labelled neurons in brainstems of mRNA-GFP and mRNA-IL10 animals. Scale bar=100 μm. FIG. 5F depicts representative quantification of retrogradely labelled neurons in the brainstem. FIG. 5G depicts representative quantification of retrogradely labelled neurons in the motor cortex. FIG. 5H depicts representative quantification of retrogradely labelled neurons in various spinal segments rostrally from the contusion injury. For FIGS. 5B, 5C and 5F-5G, data analyzed using one-way ANOVA with LSD multiple comparisons test; data presented as mean±s.e.m.; *, p<0.005 between SCI/mRNA-GFP compared to mRNA-hIL/hIL-10 osmotic pump groups; n=4.

FIG. 6, comprising FIG. 6A through FIG. 6J, depicts representative microglia/macrophage and cytokine changes after treatment with mRNA LNP encoding hIL-10 in injured spinal cords. FIG. 6A depicts representative images of parasagittal spinal cord sections 1 day after intraspinal delivery of saline, mRNA-LNP encoding eGFP, or mRNA-LNP encoding hIL-10 in the lesion area. FIG. 6B depicts representative images of parasagittal spinal cord sections 2 days after intraspinal delivery of saline, mRNA-LNP encoding eGFP, or mRNA-LNP encoding hIL-10 in the lesion area. FIG. 6A depicts representative images of parasagittal spinal cord sections 5 days after intraspinal delivery of saline, mRNA-LNP encoding eGFP, or mRNA-LNP encoding hIL-10 in the lesion area. FIG. 6D depicts representative quantification of GSA-B4 reactivity in the sagittal sections of the spinal cord 1 day after injection, demonstrating a significantly decreased level of GSA-B4 in the hIL-10 mRNA treated group compared with SCI and mRNA-GFP groups. FIG. 6E depicts representative quantification of GSA-B4 reactivity in the sagittal sections of the spinal cord 2 days after injection, demonstrating a significantly decreased level of GSA-B4 in the hIL-10 mRNA treated group compared with SCI and mRNA-GFP groups. FIG. 6F depicts representative quantification of GSA-B4 reactivity in the sagittal sections of the spinal cord 5 days after injection, demonstrating a significantly decreased level of GSA-B4 in the hIL-10 mRNA treated group compared with SCI and mRNA-GFP groups. FIG. 6G depicts representative quantification of sICAM-1 cytokine changes in injured spinal cords. FIG. 6H depicts representative quantification of CXCL7 cytokine changes in injured spinal cords. FIG. 6I depicts representative quantification of TIMP-1 cytokine changes in injured spinal cords. FIG. 6J depicts representative quantification of CNTF cytokine changes in injured spinal cords. Changes were assessed by Proteome Profiler array (ARY008), which compares the relative levels of 29 cytokines. The protein levels were normalized to time-matched injured (SCI) group. Data presented as mean s.e.m.; *p<0.05 between SCI/mRNA-GFP vs. mRNA-hIL-10; n=4. Abbreviations are as follow: REF, reference control; sICAM-1, soluble intercellular adhesion molecule-1; CXCL7, Chemokine (C—X—C motif) ligand 7; TIMP1, tissue inhibitor of matrix metalloproteinase 1; CNTF, ciliary neurotrophic factor. Grey lines (in FIG. 6G, FIG. 6H, FIG. 6I and FIG. 6J) show the injured (SCI) group value. For FIGS. 6A-6C, scale bar=300 μm.

FIG. 7, comprising FIG. 7A through FIG. 7D, depicts a schematic depicting the short and long experiments and protocols applied. Intact adult female rats (220 to 250 g) were injected with mRNA-LNP encoding eGFP or received contusion injury at the T10 vertebral level. At 7 days after the injury, a single dose of mRNA-LNPs (3.0 μg), or control saline, were administered to the injured rat spinal cords. FIG. 7A depicts the timeline for intact rats, with spinal cords collected at 2, 5, 9, 14, and 21 days after the injection. FIG. 7B depicts the timeline for injured rats that received eGFP mRNA-LNP, with spinal cords collected at 1, 2, 5, 9, 14, and 21 days after the injection. Immunohistochemistry (IHC) was performed to study eGFP expression at various timepoints. FIG. 7C depicts a timeline for another group of rats which received mRNA-LNP-encoding hIL-10 1 week after the injury. The hIL-10 expression was analyzed 1, 2, 5 and 9 days after mRNA-LNP injection. Other rats were used for various assays to analyze the acute inflammatory response and hIL-10 expression. FIG. 7D depicts a timeline for the long term study, where open field locomotor test (BBB) was applied 3 days post-SCI, followed by weekly assessment up to 9 weeks (SCI, mRNA-GFP, mRNA-hIL-10 and hIL-10 osmotic pump group). Gait parameters were measured with a plexiglass runway equipped with a mirror system to be able to record the position of the hind limb from both lateral and rear-view aspects at 9 weeks after the injury. Retrograde labelling from the L3 spinal segment and morphological analysis were performed to identify the number the retrogradely labelled neurons rostral to the injury and quantify the tissue preservation at the lesion site.

FIG. 8, comprising FIG. 8A through FIG. 8O, depicts representative imaging of eGFP expression in intact rat spinal cord. FIG. 8A depicts representative imaging of eGFP expression and rat astrocytes 2 days after injection. FIG. 8B depicts representative imaging of eGFP expression and rat microglia/macrophages 2 days after injection. FIG. 8C depicts representative imaging of eGFP expression and rat neurons 5 days after injection. FIG. 8D depicts representative imaging of eGFP expression and rat astrocytes 5 days after injection. FIG. 8E depicts representative imaging of eGFP expression and rat microglia/macrophages 5 days after injection. FIG. 8F depicts representative imaging of eGFP expression and rat neurons 5 days after injection. FIG. 8G depicts representative imaging of eGFP expression and rat astrocytes 9 days after injection. FIG. 8H depicts representative imaging of eGFP expression and rat microglia/macrophages 9 days after injection. FIG. 8I depicts representative imaging of eGFP expression and rat neurons 9 days after injection. FIG. 8J depicts representative imaging of eGFP expression and rat astrocytes 14 days after injection. FIG. 8K depicts representative imaging of eGFP expression and rat microglia/macrophages 14 days after injection. FIG. 8L depicts representative imaging of eGFP expression and rat neurons 14 days after injection. FIG. 8M depicts representative imaging of eGFP expression and rat astrocytes 21 days after injection. FIG. 8N depicts representative imaging of eGFP expression and rat microglia/macrophages 21 days after injection. FIG. 8O depicts representative imaging of eGFP expression and rat neurons 21 days after injection. Arrows show cells co-localized with eGFP. Scale bar=50 μm.

FIG. 9, comprising FIG. 9A through FIG. 9O, depicts representative imaging of eGFP expression in injured rat spinal cord. FIG. 9A depicts representative imaging of eGFP expression and rat astrocytes 2 days after injection. FIG. 9B depicts representative imaging of eGFP expression and rat microglia/macrophages 2 days after injection. FIG. 9C depicts representative imaging of eGFP expression and rat neurons 5 days after injection. FIG. 99D depicts representative imaging of eGFP expression and rat astrocytes 5 days after injection. FIG. 9E depicts representative imaging of eGFP expression and rat microglia/macrophages 5 days after injection. FIG. 9F depicts representative imaging of eGFP expression and rat neurons 5 days after injection. FIG. 9G depicts representative imaging of eGFP expression and rat astrocytes 9 days after injection. FIG. 9H depicts representative imaging of eGFP expression and rat microglia/macrophages 9 days after injection. FIG. 9I depicts representative imaging of eGFP expression and rat neurons 9 days after injection. FIG. 9J depicts representative imaging of eGFP expression and rat astrocytes 14 days after injection. FIG. 9K depicts representative imaging of eGFP expression and rat microglia/macrophages 14 days after injection. FIG. 9L depicts representative imaging of eGFP expression and rat neurons 14 days after injection. FIG. 9M depicts representative imaging of eGFP expression and rat astrocytes 21 days after injection. FIG. 9N depicts representative imaging of eGFP expression and rat microglia/macrophages 21 days after injection. FIG. 9O depicts representative imaging of eGFP expression and rat neurons 21 days after injection. Arrows show cells co-localized with eGFP. Scale bar=50 μm.

FIG. 10, comprising FIG. 10A and FIG. 10B, depicts representative hIL-10 expression in injured rat spinal cord 2 and 5 days after intraspinal administration of mRNA-LNP encoding hIL-10. FIG. 10A depicts representative confocal images show hIL-10 expression 2 days after intraspinal LNP administration in astrocytes, in GSA-B4-positive cells and in neurons. FIG. 10B depicts representative confocal images five days after the intraspinal LNP delivery hIL-10-positive neurons and astrocytes in rat spinal cord. Arrows show co-localized cells. Scale bar=25 μm.

FIG. 11, comprising FIG. 11A though FIG. 111B, depicts representative circulating cytokine changes in serum after intraspinal administration of mRNA-LNPs. Rat plasma cytokine changes were assessed with Proteome Profiler array, which compares the relative levels of 29 cytokines. The protein levels were normalized to a time-matched injured (SCI) group. FIG. 11A depicts representative imaging and quantification of CINC-1, which showed a marked decrease on day 2. FIG. 10B depicts representative quantification of other evaluated cytokines. In FIGS. 11A and 11B, grey lines show the injured (SCI) group value.

FIG. 12, comprising FIG. 12A through FIG. 12F, depicts representative expression of hGDNF, hIL-6, hIL-10, and hMIP-1α expression in injured spinal cords following intralesional delivery of mRNA-LNP. FIG. 12A depicts representative images demonstrating that none of the four factors were detected in control injured rat spinal cord (SCI). FIG. 12B depicts representative imaging demonstrating that hGDNF, hIL-6, hIL-10, and hMIP-1α protein expression is detected in paramedian sagittal sections of rat spinal cords 1 day after intraspinal delivery of mRNA-LNP. FIG. 12C depicts representative images of CCL3/MIPM-1α expression in astrocytes. FIG. 12D depicts representative images of IL-10 expression in neurons. FIG. 12E depicts representative imaging demonstrating IL-6 expression in neurons. FIG. 12F depicts representative images of GDNF expression in microglia/macrophages. For FIGS. 12C-12F, arrows show co-localized cells. For FIGS. 12A and 12B, scale bar=1 mm. For FIGS. 12C, 12D, and 12F, scale bar=50 μm. For FIG. 12E, scale bar=25 μm.

FIG. 13, comprising FIG. 13A through FIG. 13D, depicts representative improvement in locomotor function upon delayed intraspinal administration of mRNA-LNP encoding hGDNF, hIL-10, hIL-6, and hMIP-1α. FIG. 13A depicts representative open field locomotor test (BBB) results demonstrating significant improvement of the 4-factor treated animals compared with controls. FIG. 13B depicts representative quantification of toe-off angle (TOA) kinematic analysis of the animals in the various groups 9 weeks after injury. FIG. 13C depicts representative quantification of flexion and lifting kinematic analysis of the animals in the various groups 9 weeks after injury. FIG. 13D depicts representative quantification of metatarsus- (MSA) and tibia-surface angles (TSA) kinematic analysis of the animals in the various groups 9 weeks after injury.

### DETAILED DESCRIPTION

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the application, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translation by translational machinery in a cell. For example, an mRNA where all of the uridines have been replaced with pseudouridine, 1-methyl psuedouridine, or another modified nucleoside.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

In certain embodiments, “pseudouridine” refers, in another embodiment, to m1acp3Y (1-methyl-3-(3-amino-3-carboxypropyl) pseudouridine. In another embodiment, the term refers to m1Y (1-methylpseudouridine). In another embodiment, the term refers to Ym (2′-O-methylpseudouridine). In another embodiment, the term refers to m5D (5-methyldihydrouridine). In another embodiment, the term refers to m3Y (3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

The term “therapeutic” as used herein means a treatment and/or prophylaxis. A therapeutic effect is obtained by suppression, diminution, remission, or eradication of at least one sign or symptom of a disease or disorder.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

“Alkyl” refers to a straight or branched hydrocarbon chain radical consisting solely of carbon and hydrogen atoms, which is saturated or unsaturated (i.e., contains one or more double and/or triple bonds), having from one to twenty-four carbon atoms (C1-C24 alkyl), one to twelve carbon atoms (C1-C12 alkyl), one to eight carbon atoms (C1-C8 alkyl) or one to six carbon atoms (C1-C6 alkyl) and which is attached to the rest of the molecule by a single bond, e.g., methyl, ethyl, n propyl, 1-methylethyl (iso propyl), n butyl, n pentyl, 1,1 dimethylethyl (t butyl), 3 methylhexyl, 2 methylhexyl, ethenyl, prop 1 enyl, but-1-enyl, pent-1-enyl, penta-1,4-dienyl, ethynyl, propynyl, butynyl, pentynyl, hexynyl, and the like. Unless specifically stated otherwise, an alkyl group is optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double (alkenylene) and/or triple bonds (alkynylene)), and having, for example, from one to twenty-four carbon atoms (C1-C24 alkylene), one to fifteen carbon atoms (C1-C15 alkylene), one to twelve carbon atoms (C1-C12 alkylene), one to eight carbon atoms (C1-C8 alkylene), one to six carbon atoms (C1-C6 alkylene), two to four carbon atoms (C2-C4 alkylene), one to two carbon atoms (C1-C2 alkylene), e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7 dimethyl bicyclo[2.2.1]heptanyl, and the like. Unless specifically stated otherwise, a cycloalkyl group is optionally substituted.

“Cycloalkylene” is a divalent cycloalkyl group. Unless otherwise stated specifically in the specification, a cycloalkylene group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless specifically stated otherwise, a heterocyclyl group may be optionally substituted.

The term “substituted” used herein means any of the above groups (e.g., alkyl, cycloalkyl or heterocyclyl) wherein at least one hydrogen atom is replaced by a bond to a non-hydrogen atoms such as, but not limited to: a halogen atom such as F, Cl, Br, and I; oxo groups (═O); hydroxyl groups (—OH); alkoxy groups (—ORa, where Ra is C1-C12 alkyl or cycloalkyl); carboxyl groups (—OC(═O)Ra or —C(═O)ORa, where Ra is H, C1-C12 alkyl or cycloalkyl); amine groups (—NRaRb, where Ra and Rb are each independently H, C1-C12 alkyl or cycloalkyl); C1-C12 alkyl groups; and cycloalkyl groups. In some embodiments the substituent is a C1-C12 alkyl group. In other embodiments, the substituent is a cycloalkyl group. In other embodiments, the substituent is a halo group, such as fluoro. In other embodiments, the substituent is a oxo group. In other embodiments, the substituent is a hydroxyl group. In other embodiments, the substituent is an alkoxy group. In other embodiments, the substituent is a carboxyl group. In other embodiments, the substituent is an amine group.

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Messenger RNA (mRNA)-based therapy has recently emerged as a safe and very efficient approach that has wide applicability ranging from vaccination through protein replacement to gene editing (Sahin et al., 2014, Pardi et al., 2017a, Pardi et al., 2017b, Mulligan et al., 2020, Corbett et al., 2020, Laczkó et al., 2020). mRNA-based therapy has several conceptual advantages over protein or other nucleic acid-based approaches. The most advanced mRNA delivery platform utilizes lipid nanoparticle (LNP)-encapsulated nucleoside-modified mRNA. Modification of the mRNA reduces inflammatory responses after mRNA delivery and increases protein production from mRNA. LNP serves as an efficient carrier molecule for in vivo mRNA delivery (Pardi 2015) that protects mRNA from rapid degradation and facilitates its cellular uptake (Cullis 2017).

Disclosed herein are compositions and methods for modulating a microenvironment of an injured spinal cord in a subject. For example, in some embodiments, the compositions and methods described herein are useful for promoting neuroprotection and functional recovery after spinal cord injury in a subject.

In some embodiments, a composition comprises a nucleic acid molecule encoding an anti-inflammatory protein, where the neuroprotective protein induces neuroprotection and addresses issues associated with secondary spinal cord injury. In one embodiment, the composition of the invention comprises in vitro transcribed (IVT) RNA. For example, in some embodiments, the composition comprises IVT RNA which encodes a neuroprotective protein, where the neuroprotective protein induces neuroprotection of the injured spinal cord in the subject.

In some embodiments, the neuroprotective protein-encoding nucleic acid is a nucleoside-modified RNA. The invention is based in part on the finding that nucleoside-modified RNA encoding a neuroprotective protein (e.g., IL-10) can induce neuroprotection and promote functional recovery after spinal cord injury in a subject.

In some embodiments, the neuroprotective protein-encoding nucleic acid of the present composition is a purified nucleoside-modified RNA. For example, in some embodiments, the composition is purified such that it is free of double-stranded contaminants.

The composition further comprises a lipid nanoparticle (LNP). For example, in one embodiment, the composition comprises a neuroprotective protein-encoding nucleic acid molecule encapsulated within a LNP. In some instances, the LNP enhances cellular uptake of the nucleic acid molecule.

In one embodiment, the present invention provides a method for inducing neuroprotection and promoting functional recovery after spinal cord injury in a subject. In one embodiment, the method comprises treating or preventing spinal cord injury in a subject. In some embodiments, the method comprises administering to the subject a composition comprising one or more nucleoside-modified RNA encoding one or more neuroprotective proteins.

In some embodiments, the method comprises administering a plurality of doses to the subject. In another embodiment, the method comprises administering a single dose of the composition, where the single dose is effective in inducing neuroprotection and promoting functional recovery after spinal cord injury in a subject.

In some embodiments a composition comprises a nucleoside-modified mRNA encapsulated in a lipid nanoparticle wherein the nucleoside-modified mRNA encodes for a neuroprotective protein such as IL10, IL-6, MIP1a, GDNF (glial cell line-derived neurotrophic factor), or a combination thereof.

A nucleoside-modified mRNA such as the human (h)IL10-encoding mRNA, when encapsulated in a lipid nanoparticle (LNP) and intraspinally applied, was shown to translate transiently in the injured spinal cord and induce significant neuroprotection and locomotor function recovery. This opens new avenues for mRNA-mediated gene transfer for tissue engineering in the central nervous system by precisely modulating cell behaviour and function after spinal cord injury.

### Neuroprotective Protein

Exemplary neuroprotective proteins include IL10 (IL-10), IL-6 (IL-6), MIP1a, GDNF (glial cell line-derived neurotrophic factor), a combination of one or more of the foregoing, or fragment or variant thereof. Specifically contemplated combinations include human IL-10 (hIL-10) and human IL-6 (hIL-6), human IL-10 and human MIP1a, as well as human IL-10 and GDNF.

In one embodiment, the composition comprises a nucleoside-modified RNA comprising a nucleic acid sequence encoding hIL-10 comprising an amino acid sequence comprising SEQ ID NO: 1, or a fragment or variant thereof, wherein the nucleic acid sequence comprises at least one modified nucleoside.

In one embodiment, the amino acid sequence of human hIL-10 comprises:

(SEQ ID NO: 1)
MHSSALLCCLVLLTGVRASPGQGTQSENSCTHFPGNLPNMLRDLRDAFSR
VKTFFQMKDQLDNLLLKESLLEDFKGYLGCQALSEMIQFYLEEVMPQAEN
QDPDIKAHVNSLGENLKTLRLRLRRCHRFLPCENKSKAVEQVKNAFNKLQ
EKGIYKAMSEFDIFINYIEAYMTMKIRN.

In one embodiment, the composition comprises a nucleoside-modified RNA comprising a nucleic acid sequence encoding hIL-6 comprising an amino acid sequence comprising SEQ ID NO: 2, or a fragment or variant thereof, wherein the nucleic acid sequence comprises at least one modified nucleoside.

In one embodiment, the amino acid sequence of human hIL-6 comprises:

(SEQ ID NO: 2)
MNSFSTSAFGPVAFSLGLLLVLPAAFPAPVPPGEDSKDVAAPHRQPLTSS
ERIDKQIRYILDGISALRKETCNKSNMCESSKEALAENNLNLPKMAEKDG
CFQSGFNEETCLVKIITGLLEFEVYLEYLQNRFESSEEQARAVQMSTKVL
IQFLQKKAKNLDAITTPDPTTNASLLTKLQAQNQWLQDMTTHLILRSFKE
FLQSSLRALRQM.

In one embodiment, the composition comprises a nucleoside-modified RNA comprising a nucleic acid sequence encoding MIP1a comprising an amino acid sequence comprising SEQ ID NO: 3, or a fragment or variant thereof, wherein the nucleic acid sequence comprises at least one modified nucleoside.

In one embodiment, the amino acid sequence of human MIP1a comprises:

(SEQ ID NO: 3)
MQVSTAALAVLLCTMALCNQFSASLAADTPTACCFSYTSRQIPQNFIADY
FETSSQCSKPGVIFLTKRSRQVCADPSEEWVQKYVSDLELSA.

In one embodiment, the composition comprises a nucleoside-modified RNA comprising a nucleic acid sequence encoding GDNF comprising an amino acid sequence comprising SEQ ID NO: 4, or a fragment or variant thereof, wherein the nucleic acid sequence comprises at least one modified nucleoside.

In one embodiment, the amino acid sequence of GDNF comprises:

(SEQ ID NO: 4)
MKLWDVVAVCLVLLHTASAFPLPAGKRPPEAPAEDRSLGRRRAPFALSSD
SNMPEDYPDQFDDVMDFIQATIKRLKRSPDKQMAVLPRRERNRQAAAANP
ENSRGKGRRGQRGKNRGCVLTAIHLNVTDLGLGYETKEELIFRYCSGSCD
AAETTYDKILKNLSRNRRLVSDKVGQACCRPIAFDDDLSFLDDNLVYHIL
RKHSAKRCGCI.

In one embodiment, the composition comprises a nucleoside-modified RNA comprising a nucleic acid sequence encoding hIL-10, wherein the nucleic acid sequence is encoded by a DNA sequence comprising SEQ ID NO: 5, or a fragment or variant thereof, wherein the nucleic acid sequence comprises at least one modified nucleoside.

In certain embodiments, the nucleoside-modified RNA encoding hIL-10 is encoded by an optimized DNA sequence. In one embodiment, the DNA sequence encoding the nucleoside-modified RNA encoding hIL-10, comprises the nucleotide sequence of:

(SEQ ID NO: 5)
ATGCACTCCTCCGCCCTGCTGTGCTGCCTGGTGCTGCTGACCGGCGTGCG
CGCCTCCCCCGGCCAGGGCACCCAGTCCGAGAACTCCTGCACCCACTTCC
CCGGCAACCTGCCCAACATGCTGCGCGACCTGCGCGACGCCTTCTCCCGC
GTGAAGACCTTCTTCCAGATGAAGGACCAGCTGGACAACCTGCTGCTGAA
GGAGTCCCTGCTGGAGGACTTCAAGGGCTACCTGGGCTGCCAGGCCCTGT
CCGAGATGATCCAGTTCTACCTGGAGGAGGTGATGCCCCAGGCCGAGAAC
CAGGACCCCGACATCAAGGCCCACGTGAACTCCCTGGGCGAGAACCTGAA
GACCCTGCGCCTGCGCCTGCGCCGCTGCCACCGCTTCCTGCCCTGCGAGA
ACAAGTCCAAGGCCGTGGAGCAGGTGAAGAACGCCTTCAACAAGCTGCAG
GAGAAGGGCATCTACAAGGCCATGTCCGAGTTCGACATCTTCATCAACTA
CATCGAGGCCTACATGACCATGAAGATCCGCAAC.

In one embodiment, the composition comprises a nucleoside-modified RNA comprising a nucleic acid sequence encoding hIL-6, wherein the nucleic acid sequence is encoded by a DNA sequence comprising SEQ ID NO: 6, or a fragment or variant thereof, wherein the nucleic acid sequence comprises at least one modified nucleoside.

In certain embodiments, the nucleoside-modified RNA encoding hIL-6 is encoded by an optimized DNA sequence. In one embodiment, the DNA sequence encoding the nucleoside-modified RNA encoding hIL-6, comprises the nucleotide sequence of:

(SEQ ID NO: 6)
ATGAACTCCTTCTCCACCTCCGCCTTCGGCCCCGTGGCCTTCTCCCTGGG
CCTGCTGCTGGTGCTGCCCGCCGCCTTCCCCGCCCCCGTGCCCCCCGGCG
AGGACTCCAAGGACGTGGCCGCCCCCCACCGCCAGCCCCTGACCTCCTCC
GAGCGCATCGACAAGCAGATCCGCTACATCCTGGACGGCATCTCCGCCCT
GCGCAAGGAGACCTGCAACAAGTCCAACATGTGCGAGTCCTCCAAGGAGG
CCCTGGCCGAGAACAACCTGAACCTGCCCAAGATGGCCGAGAAGGACGGC
TGCTTCCAGTCCGGCTTCAACGAGGAGACCTGCCTGGTGAAGATCATCAC
CGGCCTGCTGGAGTTCGAGGTGTACCTGGAGTACCTGCAGAACCGCTTCG
AGTCCTCCGAGGAGCAGGCCCGCGCCGTGCAGATGTCCACCAAGGTGCTG
ATCCAGTTCCTGCAGAAGAAGGCCAAGAACCTGGACGCCATCACCACCCC
CGACCCCACCACCAACGCCTCCCTGCTGACCAAGCTGCAGGCCCAGAACC
AGTGGCTGCAGGACATGACCACCCACCTGATCCTGCGCTCCTTCAAGGAG
TTCCTGCAGTCCTCCCTGCGCGCCCTGCGCCAGATG

In one embodiment, the composition comprises a nucleoside-modified RNA comprising a nucleic acid sequence encoding human MIP1a, wherein the nucleic acid sequence is encoded by a DNA sequence comprising SEQ ID NO: 7, or a fragment or variant thereof, wherein the nucleic acid sequence comprises at least one modified nucleoside.

In certain embodiments, the nucleoside-modified RNA encoding human MIP1a is encoded by an optimized DNA sequence. In one embodiment, the DNA sequence encoding the nucleoside-modified RNA encoding human MIP1a, comprises the nucleotide sequence of:

(SEQ ID NO: 7)
ATGCAGGTGTCCACCGCCGCCCTGGCCGTGCTGCTGTGCACCATGGCCCT
GTGCAACCAGTTCTCCGCCTCCCTGGCCGCCGACACCCCCACCGCCTGCT
GCTTCTCCTACACCTCCCGCCAGATCCCCCAGAACTTCATCGCCGACTAC
TTCGAGACCTCCTCCCAGTGCTCCAAGCCCGGCGTGATCTTCCTGACCAA
GCGCTCCCGCCAGGTGTGCGCCGACCCCTCCGAGGAGTGGGTGCAGAAGT
ACGTGTCCGACCTGGAGCTGTCCGCC.

In one embodiment, the composition comprises a nucleoside-modified RNA comprising a nucleic acid sequence encoding GDNF, wherein the nucleic acid sequence is encoded by a DNA sequence comprising SEQ ID NO: 8, or a fragment or variant thereof, wherein the nucleic acid sequence comprises at least one modified nucleoside.

In certain embodiments, the nucleoside-modified RNA encoding GDNF is encoded by an optimized DNA sequence. In one embodiment, the DNA sequence encoding the nucleoside-modified RNA encoding GDNF, comprises the nucleotide sequence of.

(SEQ ID NO: 8)
ATGAAGCTGTGGGACGTGGTGGCCGTGTGCCTGGTGCTGCTGCACACCGC
CTCCGCCTTCCCCCTGCCCGCCGGCAAGCGCCCCCCCGAGGCCCCCGCCG
AGGACCGCTCCCTGGGCCGCCGCCGCGCCCCCTTCGCCCTGTCCTCCGAC
TCCAACATGCCCGAGGACTACCCCGACCAGTTCGACGACGTGATGGACTT
CATCCAGGCCACCATCAAGCGCCTGAAGCGCTCCCCCGACAAGCAGATGG
CCGTGCTGCCCCGCCGCGAGCGCAACCGCCAGGCCGCCGCCGCCAACCCC
GAGAACTCCCGCGGCAAGGGCCGCCGCGGCCAGCGCGGCAAGAACCGCGG
CTGCGTGCTGACCGCCATCCACCTGAACGTGACCGACCTGGGCCTGGGCT
ACGAGACCAAGGAGGAGCTGATCTTCCGCTACTGCTCCGGCTCCTGCGAC
GCCGCCGAGACCACCTACGACAAGATCCTGAAGAACCTGTCCCGCAACCG
CCGCCTGGTGTCCGACAAGGTGGGCCAGGCCTGCTGCCGCCCCATCGCCT
TCGACGACGACCTGTCCTTCCTGGACGACAACCTGGTGTACCACATCCTG
CGCAAGCACTCCGCCAAGCGCTGCGGCTGCATC.

In some embodiments, the neuroprotective protein comprises an amino acid sequence that is substantially homologous to the amino acid sequence of a neuroprotective protein described herein and retains the function of the original amino acid sequence. For example, in some embodiments, the amino acid sequence of the neuroprotective protein has a degree of identity with respect to the original neuroprotective protein sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In one embodiment, the neuroprotective protein is encoded by a nucleic acid sequence of a nucleic acid molecule. In some embodiments, the nucleic acid sequence comprises DNA, RNA, cDNA, viral DNA, a variant thereof, a fragment thereof, or a combination thereof. In one embodiment, the nucleic acid sequence comprises a modified nucleic acid sequence. For example, in one embodiment the neuroprotective protein-encoding nucleic acid sequence comprises nucleoside-modified RNA, as described in detail elsewhere herein.

### Nucleic Acids

In one embodiment, the invention includes a nucleic acid molecule encoding a neuroprotective protein, or a fragment or variant thereof. In one embodiment, the invention includes a nucleoside-modified nucleic acid molecule. In one embodiment, the nucleoside-modified nucleic acid molecule encodes neuroprotective protein, or a fragment or variant thereof. In one embodiment, the nucleoside-modified nucleic acid molecule encodes a plurality of neuroprotective proteins, or a fragments or variants thereof.

In one embodiment, the invention includes one or more nucleic acid molecules encoding a neuroprotective protein, fragment, or variant thereof. In one embodiment, the one or more nucleic acid molecules comprise one or more nucleotide sequence selected from the group consisting of SEQ ID NOs: 5-8, sequences substantially homologous to SEQ ID NOs: 5-8, variants of SEQ ID NOs: 5-8, and fragments of SEQ ID NOs:5-8. In one embodiment, the nucleic acid molecule comprises a nucleotide sequence which encodes one or more amino acid sequence selected from the group consisting of SEQ ID NOs: 1-4, sequences substantially homologous to SEQ ID NOs: 1-4, variants of SEQ ID NOs:1-4, and fragments of SEQ ID NOs:1-4.

The nucleic acid molecule can be made using any methodology in the art, including, but not limited to, in vitro transcription, chemical synthesis, or the like.

The nucleotide sequences encoding a neuroprotective protein, or a fragment or variant thereof, as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous to the nucleotide sequences recited herein and encode a neuroprotective protein of interest, or a fragment or variant thereof.

As used herein, a nucleotide sequence is “substantially homologous” to any of the nucleotide sequences described herein when its nucleotide sequence has a degree of identity with respect to the original nucleotide sequence at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. A nucleotide sequence that is substantially homologous to a nucleotide sequence encoding a neuroprotective protein can typically be isolated from a producer organism of the neuroprotective protein based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art.

Further, the scope of the invention includes nucleotide sequences that encode amino acid sequences that are substantially homologous to the amino acid sequences recited herein and preserve the function of the original amino acid sequence.

As used herein, an amino acid sequence is “substantially homologous” to any of the amino acid sequences described herein when its amino acid sequence has a degree of identity with respect to the original amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. The identity between two amino acid sequences can be determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

In one embodiment, the invention relates to a construct, comprising a nucleotide sequence encoding a neuroprotective protein, or a fragment or variant thereof. In one embodiment, the construct comprises a plurality of nucleotide sequences encoding a plurality of neuroprotective proteins, or a fragments or variants thereof. For example, in some embodiments, the construct encodes 1 or more, 2 or more, 3 or more, 4 or more, 5 or more, or 10 or more neuroprotective proteins, or a fragments or variants thereof. In one embodiment, the invention relates to a construct, comprising a nucleotide sequence encoding an adjuvant.

In one embodiment, the composition comprises a plurality of constructs, each construct encoding one or more a neuroprotective proteins, or a fragments or variants thereof. In some embodiments, the composition comprises 1 or more, 2 or more, 3 or more, 4 or more, 5 or more, 6 or more, 7 or more, 8 or more, 9 or more, 10 or more, 15 or more, or 20 or more constructs. In one embodiment, the composition comprises a first construct, comprising a nucleotide sequence encoding a first neuroprotective protein; and a second construct, comprising a nucleotide sequence encoding a second neuroprotective protein.

In another particular embodiment, the construct is operatively bound to a translational control element. The construct can incorporate an operatively bound regulatory sequence for the expression of the nucleotide sequence of the invention, thus forming an expression cassette.

### In Vitro Transcribed RNA

In one embodiment, the composition of the invention comprises in vitro transcribed (IVT) RNA encoding a neuroprotective protein, or a fragment or variant thereof. In one embodiment, the composition of the invention comprises IVT RNA encoding a plurality of neuroprotective proteins, or fragments or variants thereof.

In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In one embodiment, the desired template for in vitro transcription is a neuroprotective protein, or a fragment or variant thereof.

In one embodiment, the DNA to be used for PCR contains an open reading frame. The DNA can be from a naturally occurring DNA sequence from the genome of an organism. In one embodiment, the DNA is a full-length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. In another embodiment, the DNA to be used for PCR is a gene from a pathogenic or commensal organism, including bacteria, viruses, parasites, and fungi. In another embodiment, the DNA to be used for PCR is from a pathogenic or commensal organism, including bacteria, viruses, parasites, and fungi, including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that prevent inflammation in an organism. In some instances, the genes are useful for a short term treatment. In some instances, the genes have limited safety concerns regarding dosage of the expressed gene.

In various embodiments, a plasmid is used to generate a template for in vitro transcription of mRNA, which is used for transfection.

Chemical structures with the ability to promote stability and/or translation efficiency may also be used. In some embodiments, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability of mRNA in the cell. On a circular DNA template, for instance, plasmid DNA, RNA polymerase produces a long concatameric product, which is not suitable for expression in eukaryotic cells. The transcription of plasmid DNA linearized at the end of the 3′ UTR results in normal sized mRNA, which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003)).

The conventional method of integration of polyA/T stretches into a DNA template is molecular cloning. However, polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to mRNA molecules. In one embodiment, RNAs produced by the methods to include a 5′ cap1 structure. Such cap1 structure can be generated using Vaccinia capping enzyme and 2′-O-methyltransferase enzymes (CellScript, Madison, WI). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)). In some embodiments RNA of the invention is introduced to a cell with a method comprising the use of TransIT®-mRNA transfection Kit (Mirus, Madison WI), which, in some instances, provides high efficiency, low toxicity, transfection.

### Nucleoside-Modified RNA

The composition disclosed herein comprises a nucleoside-modified nucleic acid encoding a neuroprotective protein, or a fragment or variant thereof as described herein. In one embodiment, the composition of the present invention comprises a nucleoside-modified nucleic acid encoding a plurality of neuroprotective proteins, or fragments or variants thereof.

For example, in one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. Nos. 8,278,036, 8,691,966, and 8,835,108, each of which is incorporated by reference herein in its entirety.

In some embodiments, nucleoside-modified mRNA does not activate any pathophysiologic pathways, translates very efficiently and almost immediately following delivery, and serve as templates for continuous protein production in vivo lasting for several days to weeks (Karikó et al., 2008, Mol Ther 16:1833-1840; Karikó et al., 2012, Mol Ther 20:948-953). The amount of mRNA required to exert a physiological effect is small, making it applicable for human therapy. For example, as described herein, nucleoside-modified mRNA encoding a neuroprotective protein (e.g., PDGF-BB and BMP-2) has demonstrated the ability to induce neuroprotection and functional recovery in spinal cord injuries. For example, in some instances, a neuroprotective protein encoded by nucleoside-modified mRNA induces greater effect as compared to a neuroprotective protein encoded by non-modified mRNA.

In some instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In some embodiments, using mRNA rather than the protein also has many advantages. Half-lives of proteins in the circulation or in tissues are often short, thus protein treatment would need frequent dosing, while mRNA provides a template for continuous protein production for several days to weeks. Purification of proteins is problematic and they can contain aggregates and other impurities that cause adverse effects (Kromminga and Schellekens, 2005, Ann NY Acad Sci 1050:257-265).

In some embodiments, the nucleoside-modified RNA comprises the naturally occurring modified-nucleoside pseudouridine. In some embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953; Karikó et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892).

Similar effects as described for pseudouridine have also been observed for RNA containing 1-methyl-pseudouridine.

In some embodiments, the nucleoside-modified nucleic acid molecule is a purified nucleoside-modified nucleic acid molecule. For example, in some embodiments, the composition is purified to remove double-stranded contaminants. In some instances, a preparative high-performance liquid chromatography (HPLC) purification procedure is used to obtain pseudouridine-containing RNA that has superior translational potential and no innate immunogenicity (Karikó et al., 2011, Nucleic Acids Research 39:e142). Administering HPLC-purified, pseudouridine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy. In some embodiments, the nucleoside-modified nucleic acid molecule is purified using non-HPLC methods. In some instances, the nucleoside-modified nucleic acid molecule is purified using chromatography methods, including but not limited to HPLC and fast protein liquid chromatography (FPLC). An exemplary FPLC-based purification procedure is described in Weissman et al., 2013, Methods Mol Biol, 969: 43-54. Exemplary purification procedures are also described in U.S. Patent Application Publication No. US2016/0032316, which is hereby incorporated by reference in its entirety.

The present invention encompasses RNA, oligoribonucleotide, and polyribonucleotide molecules comprising pseudouridine or a modified nucleoside. In some embodiments, the composition comprises an isolated nucleic acid encoding a neuroprotective protein, wherein the nucleic acid comprises a pseudouridine or a modified nucleoside. In some embodiments, the composition comprises a vector, comprising an isolated nucleic acid encoding neuroprotective protein, wherein the nucleic acid comprises a pseudouridine or a modified nucleoside.

In one embodiment, the nucleoside-modified RNA of the invention is IVT RNA, as described elsewhere herein. For example, in some embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In one embodiment, the modified nucleoside is m1acp3Ψ (1-methyl-3-(3-amino-3-carboxypropyl) pseudouridine. In another embodiment, the modified nucleoside is m1Ψ (1-methylpseudouridine). In another embodiment, the modified nucleoside is Ψm (2′-O-methylpseudouridine). In another embodiment, the modified nucleoside is m5D (5-methyldihydrouridine). In another embodiment, the modified nucleoside is m3Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the nucleoside that is modified in the nucleoside-modified RNA the present invention is uridine (U). In another embodiment, the modified nucleoside is cytidine (C). In another embodiment, the modified nucleoside is adenosine (A). In another embodiment, the modified nucleoside is guanosine (G).

In another embodiment, the modified nucleoside of the present invention is m5C (5-methylcytidine). In another embodiment, the modified nucleoside is m5U (5-methyluridine). In another embodiment, the modified nucleoside is m6A (N6-methyladenosine). In another embodiment, the modified nucleoside is s2U (2-thiouridine). In another embodiment, the modified nucleoside is Ψ (pseudouridine). In another embodiment, the modified nucleoside is Um (2′-O-methyluridine).

In other embodiments, the modified nucleoside is m1A (1-methyladenosine); m2A (2-methyladenosine); Am (2′-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonylcarbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyladenosine); ms2hn6A (2-methylthio-N6-hydroxynorvalyl carbamoyladenosine); Ar(p) (2′-O-ribosyladenosine (phosphate)); I (inosine); m1I (1-methylinosine); m1Im (1,2′-O-dimethylinosine); m3C (3-methylcytidine); Cm (2′-O-methylcytidine); s2C (2-thiocytidine); ac4C (N4-acetylcytidine); f5C (5-formylcytidine); m5Cm (5,2′-O-dimethylcytidine); ac4Cm (N4-acetyl-2′-O-methylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQO (7-cyano-7-deazaguanosine); preQl (7-aminomethyl-7-deazaguanosine); G+(archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonylmethyluridine); mcm5Um (5-methoxycarbonylmethyl-2′-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyluridine); ncm5Um (5-carbamoylmethyl-2′-O-methyluridine); cmnm5U (5-carboxymethylaminomethyluridine); cmnm5Um (5-carboxymethylaminomethyl-2′-O-methyluridine); cmnm5s2U (5-carboxymethylaminomethyl-2-thiouridine); m62A (N6,N6-dimethyladenosine); Im (2′-O-methylinosine); m4C (N4-methylcytidine); m4Cm (N4,2′-O-dimethylcytidine); hm5C (5-hydroxymethylcytidine); m3U (3-methyluridine); cm5U (5-carboxymethyluridine); m6Am (N6,2′-O-dimethyladenosine); m62Am (N6,N6,O-2′-trimethyladenosine); m2,7G (N2,7-dimethylguanosine); m2,2,7G (N2,N2,7-trimethylguanosine); m3Um (3,2′-O-dimethyluridine); m5D (5-methyldihydrouridine); f5Cm (5-formyl-2′-O-methylcytidine); m1Gm (1,2′-O-dimethylguanosine); m1Am (1,2′-O-dimethyladenosine); rm5U (5-taurinomethyluridine); τm5s2U (5-taurinomethyl-2-thiouridine)); imG-14 (4-demethylwyosine); imG2 (isowyosine); or ac6A (N6-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In various embodiments, between 0.1% and 100% of the residues in the nucleoside-modified RNA of the present invention are modified (e.g., either by the presence of pseudouridine, 1-methyl-pseudouridine, or another modified nucleoside base). In one embodiment, the fraction of modified residues is 0.1%. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%. In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In some embodiments, the composition comprises a purified preparation of single-stranded nucleoside modified RNA. For example, in some embodiments, the purified preparation of single-stranded nucleoside modified RNA is substantially free of double stranded RNA (dsRNA). In some embodiments, the purified preparation is at least 90%, or at least 91%, or at least 92%, or at least 93% or at least 94%, or at least 95%, or at least 96%, or at least 97%, or at least 98%, or at least 99%, or at least 99.5%, or at least 99.9% single stranded nucleoside modified RNA, relative to all other nucleic acid molecules (DNA, dsRNA, etc.).

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 4-fold factor. In another embodiment, translation is enhanced by a 5-fold factor. In another embodiment, translation is enhanced by a 6-fold factor. In another embodiment, translation is enhanced by a 7-fold factor. In another embodiment, translation is enhanced by an 8-fold factor. In another embodiment, translation is enhanced by a 9-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified RNA of the present invention exhibits significantly less innate immunogenicity than an unmodified in vitro-synthesized RNA molecule of the same sequence. In another embodiment, the modified RNA molecule exhibits an innate immune response that is 2-fold less than its unmodified counterpart. In another embodiment, innate immunogenicity is reduced by a 3-fold factor. In another embodiment, innate immunogenicity is reduced by a 4-fold factor. In another embodiment, innate immunogenicity is reduced by a 5-fold factor. In another embodiment, innate immunogenicity is reduced by a 6-fold factor. In another embodiment, innate immunogenicity is reduced by a 7-fold factor. In another embodiment, innate immunogenicity is reduced by a 8-fold factor. In another embodiment, innate immunogenicity is reduced by a 9-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the protein encoded by the modified RNA. In another embodiment, the decrease is such that the nucleoside-modified RNA can be repeatedly administered without eliciting an innate immune response sufficient to eliminate detectable production of the protein encoded by the modified RNA.

### Lipid Nanoparticle

In one embodiment, delivery of nucleoside-modified RNA comprises any suitable delivery method, including exemplary RNA transfection methods described elsewhere herein. In some embodiments, delivery of a nucleoside-modified RNA to a subject comprises mixing the nucleoside-modified RNA with a transfection reagent prior to the step of contacting. In another embodiment, a method of present invention further comprises administering nucleoside-modified RNA together with the transfection reagent. In another embodiment, the transfection reagent is a cationic lipid reagent. In another embodiment, the transfection reagent is a cationic polymer reagent.

In another embodiment, the transfection reagent is a lipid-based transfection reagent. In another embodiment, the transfection reagent is a protein-based transfection reagent. In another embodiment, the transfection reagent is a carbohydrate-based transfection reagent. In another embodiment, the transfection reagent is a cationic lipid-based transfection reagent. In another embodiment, the transfection reagent is a cationic polymer-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids, which make up the cell membrane. They have, in another embodiment, an internal aqueous space for entrapping water-soluble compounds and range in size from 0.05 to several microns in diameter. In another embodiment, liposomes can deliver RNA to cells in a biologically active form.

In one embodiment, the composition comprises a lipid nanoparticle (LNP) and one or more nucleic acid molecules described herein. For example, in one embodiment, the composition comprises an LNP and one or more nucleoside-modified RNA molecules encoding one or more neuroprotective proteins, or fragments or variants thereof.

The term “lipid nanoparticle” refers to a particle having at least one dimension on the order of nanometers (e.g., 1-1,000 nm), which includes one or more lipids, for example a lipid of Formula (I), (II) or (III). In some embodiments, lipid nanoparticles are included in a formulation comprising a nucleoside-modified RNA as described herein. In some embodiments, such lipid nanoparticles comprise a cationic lipid (e.g., a lipid of Formula (I), (II) or (III)) and one or more excipient selected from neutral lipids, charged lipids, steroids and polymer conjugated lipids (e.g., a pegylated lipid such as a pegylated lipid of structure (IV), such as compound Iva). In some embodiments, the nucleoside-modified RNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In some embodiments, the nucleoside-modified RNA, when present in the lipid nanoparticles, is resistant in aqueous solution to degradation with a nuclease.

The LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In one embodiment, the LNP comprises one or more cationic lipids, and one or more stabilizing lipids. Stabilizing lipids include neutral lipids and pegylated lipids.

In one embodiment, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In some embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

Suitable amino lipids include those having the formula:

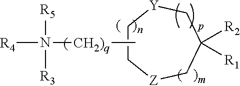

wherein R₁ and R₂ are either the same or different and independently optionally substituted C10-C24 alkyl, optionally substituted C10-C24 alkenyl, optionally substituted C10-C24 alkynyl, or optionally substituted C10-C24 acyl;
R₃ and R₄ are either the same or different and independently optionally substituted C1-C6 alkyl, optionally substituted C2-C6 alkenyl, or optionally substituted C2-C6 alkynyl or R₃ and R₄ may join to form an optionally substituted heterocyclic ring of 4 to 6 carbon atoms and 1 or 2 heteroatoms chosen from nitrogen and oxygen;
R₅ is either absent or present and when present is hydrogen or C1-C6 alkyl;
m, n, and p are either the same or different and independently either 0 or 1 with the proviso that m, n, and p are not simultaneously 0;
q is 0, 1, 2, 3, or 4; and
Y and Z are either the same or different and independently O, S, or NH.

In one embodiment, R₁ and R₂ are each linoleyl, and the amino lipid is a dilinoleyl amino lipid. In one embodiment, the amino lipid is a dilinoleyl amino lipid. A representative useful dilinoleyl amino lipid has the formula:

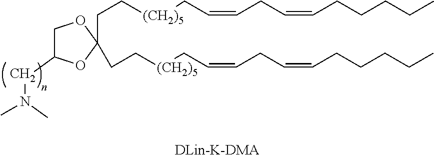

wherein n is 0, 1, 2, 3, or 4.

In one embodiment, the cationic lipid is a DLin-K-DMA. In one embodiment, the cationic lipid is DLin-KC2-DMA (DLin-K-DMA above, wherein n is 2).

In one embodiment, the cationic lipid component of the LNPs has the structure of Formula (I):

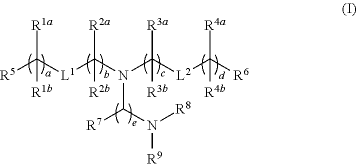

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C1-C12 alkyl, or (b) R^{1a} is H or C1-C12 alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C1-C12 alkyl, or (b) R^{2a} is H or C1-C12 alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C1-C12 alkyl, or (b) R^{3a} is H or C1-C12 alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C1-C12 alkyl, or (b) R^{4a} is H or C1-C12 alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C1-C12 alkyl;
R⁸ and R⁹ are each independently C1-C12 alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2.

In some embodiments of Formula (I), at least one of R^{1a}, R^{2a}, R^{3a} or R^{4a} is C1-C12 alkyl, or at least one of L1 or L2 is —O(C═O)— or —(C═O)O—. In other embodiments, R1a and R1b are not isopropyl when a is 6 or n-butyl when a is 8.

In still further embodiments of Formula (I), at least one of R^{1a}, R^{2a}, R^{3a} or R^{4a} is C1-C12 alkyl, or at least one of L1 or L2 is —O(C═O)— or —(C═O)O—; and

R^{1a} and R^{1b} are not isopropyl when a is 6 or n-butyl when a is 8.

In other embodiments of Formula (I), R8 and R9 are each independently unsubstituted C1-C12 alkyl; or R8 and R9, together with the nitrogen atom to which they are attached, form a 5, 6 or 7-membered heterocyclic ring comprising one nitrogen atom;

In some embodiments of Formula (I), any one of L1 or L2 may be —O(C═O)— or a carbon-carbon double bond. L1 and L2 may each be —O(C═O)— or may each be a carbon-carbon double bond.

In some embodiments of Formula (I), one of L1 or L2 is —O(C═O)—. In other embodiments, both L1 and L2 are —O(C═O)—.

In some embodiments of Formula (I), one of L1 or L2 is —(C═O)O—. In other embodiments, both L1 and L2 are —(C═O)O—.

In some other embodiments of Formula (I), one of L1 or L2 is a carbon-carbon double bond. In other embodiments, both L1 and L2 are a carbon-carbon double bond.

In still other embodiments of Formula (I), one of L1 or L2 is —O(C═O)— and the other of L1 or L2 is —(C═O)O—. In more embodiments, one of L1 or L2 is —O(C═O)— and the other of L1 or L2 is a carbon-carbon double bond. In yet more embodiments, one of L1 or L2 is —(C═O)O— and the other of L1 or L2 is a carbon-carbon double bond.

It is understood that “carbon-carbon” double bond, as used throughout the specification, refers to one of the following structures:

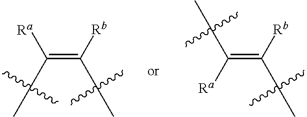

wherein Ra and Rb are, at each occurrence, independently H or a substituent. For example, in some embodiments Ra and Rb are, at each occurrence, independently H, C1-C12 alkyl or cycloalkyl, for example H or C1-C12 alkyl.

In other embodiments, the lipid compounds of Formula (I) have the following structure (Ia):

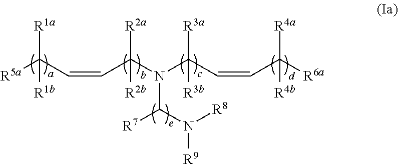

In other embodiments, the lipid compounds of Formula (I) have the following structure (1b):

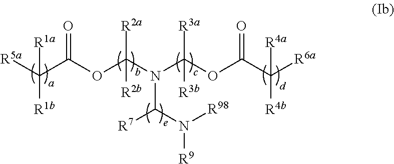

In yet other embodiments, the lipid compounds of Formula (I) have the following structure (Ic):

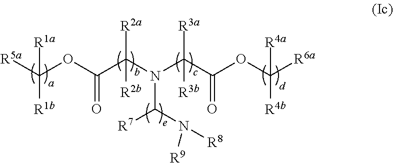

In some embodiments of the lipid compound of Formula (I), a, b, c and d are each independently an integer from 2 to 12 or an integer from 4 to 12. In other embodiments, a, b, c and d are each independently an integer from 8 to 12 or 5 to 9. In some embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some other embodiments of Formula (I), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some more embodiments of Formula (I), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some other embodiments of Formula (I), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some other various embodiments of Formula (I), a and d are the same. In some other embodiments, b and c are the same. In some other specific embodiments, a and d are the same and b and c are the same.

The sum of a and b and the sum of c and d in Formula (I) are factors which may be varied to obtain a lipid of Formula (I) having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a, b, c and d are selected such the sum of a and b and the sum of c and d is 12 or greater. In some embodiments of Formula (I), e is 1. In other embodiments, e is 2.

The substituents at R1a, R2a, R3a and R4a of Formula (I) are not particularly limited. In some embodiments R1a, R2a, R3a and R4a are H at each occurrence. In some other embodiments at least one of R1a, R2a, R3a and R4a is C1-C12 alkyl. In some other embodiments at least one of R1a, R2a, R3a and R4a is C1-C8 alkyl. In some other embodiments at least one of R1a, R2a, R3a and R4a is C1-C6 alkyl. In some of the foregoing embodiments, the C1-C8 alkyl is methyl, ethyl, n-propyl, iso-propyl, n-butyl, iso-butyl, tert-butyl, n-hexyl or n-octyl.

In some embodiments of Formula (I), R1a, R1b, R4a and R4b are C1-C12 alkyl at each occurrence.

In further embodiments of Formula (I), at least one of R1b, R2b, R3b and R4b is H or R1b, R2b, R3b and R4b are H at each occurrence.

In some embodiments of Formula (I), R1b together with the carbon atom to which it is bound is taken together with an adjacent R1b and the carbon atom to which it is bound to form a carbon-carbon double bond. In other embodiments of the foregoing R4b together with the carbon atom to which it is bound is taken together with an adjacent R4b and the carbon atom to which it is bound to form a carbon-carbon double bond.

The substituents at R5 and R6 of Formula (I) are not particularly limited in the foregoing embodiments. In some embodiments one or both of R5 or R6 is methyl. In some other embodiments one or both of R5 or R6 is cycloalkyl for example cyclohexyl. In these embodiments the cycloalkyl may be substituted or not substituted. In some other embodiments the cycloalkyl is substituted with C1-C12 alkyl, for example tert-butyl.

The substituents at R7 are not particularly limited in the foregoing embodiments of Formula (I). In some embodiments at least one R7 is H. In some other embodiments, R7 is H at each occurrence. In some other embodiments R7 is C1-C12 alkyl.

In some other of the foregoing embodiments of Formula (I), one of R8 or R9 is methyl. In other embodiments, both R8 and R9 are methyl.

In some different embodiments of Formula (I), R8 and R9, together with the nitrogen atom to which they are attached, form a 5, 6 or 7-membered heterocyclic ring. In some embodiments of the foregoing, R8 and R9, together with the nitrogen atom to which they are attached, form a 5-membered heterocyclic ring, for example a pyrrolidinyl ring.

In various different embodiments, the lipid of Formula (I) has one of the structures set forth in Table 1 below.

TABLE 1
Representative Lipids of Formula (I)

| | | Prep. |
|---|---|---|
| No. | Structure | Method |
| I-1 | 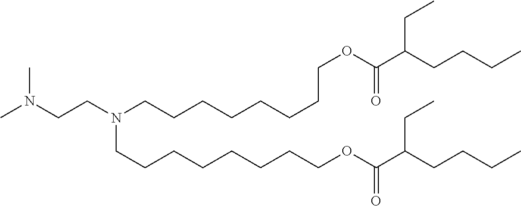 [SMILES: CCCCC(CC)C(=O)OCCCCCCCCN(CCCCCCCCOC(=O)C(CC)CCCC)CCN(C)C] | B |
| I-2 | 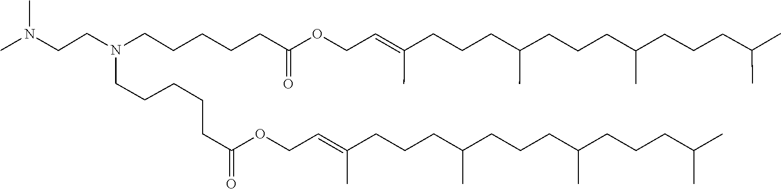 [SMILES: C/C(=C\COC(=O)CCCCCN(CCCCCC(=O)OC/C=C(\C)CCCC(C)CCCC(C)CCCC(C)C)CCN(C)C)CCCC(C)CCCC(C)CCCC(C)C] | A |
| I-3 | 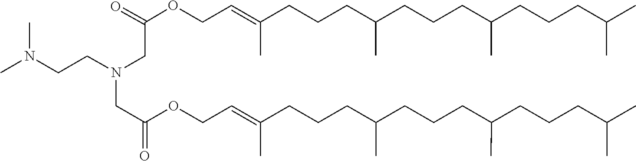 [SMILES: C/C(=C\COC(=O)CN(CCN(C)C)CC(=O)OC/C=C(\C)CCCC(C)CCCC(C)CCCC(C)C)CCCC(C)CCCC(C)CCCC(C)C] | A |
| I-4 | 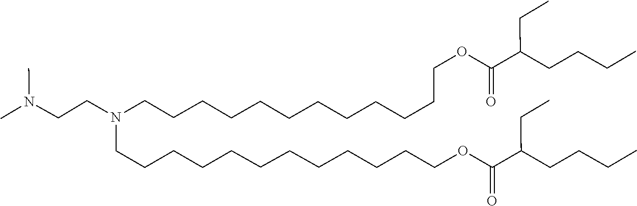 [SMILES: CCCCC(CC)C(=O)OCCCCCCCCCCCCN(CCCCCCCCCCCCOC(=O)C(CC)CCCC)CCN(C)C] | B |
| I-5 | 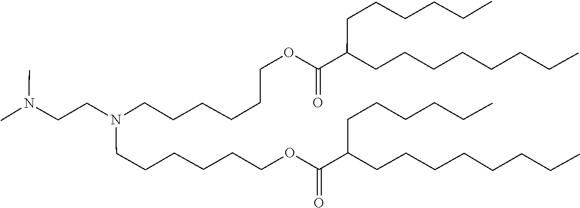 [SMILES: CCCCCCCCC(CCCCCC)C(=O)OCCCCCCN(CCCCCCOC(=O)C(CCCCCC)CCCCCCCC)CCN(C)C] | B |
| I-6 | 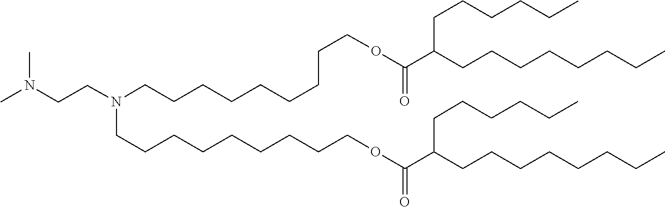 [SMILES: CCCCCCCCC(CCCCCC)C(=O)OCCCCCCCCCN(CCCCCCCCCOC(=O)C(CCCCCC)CCCCCCCC)CCN(C)C] | B |
| I-7 | 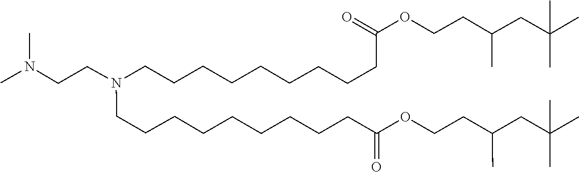 [SMILES: CC(CCOC(=O)CCCCCCCCCN(CCCCCCCCCC(=O)OCCC(C)CC(C)(C)C)CCN(C)C)CC(C)(C)C] | A |
| I-8 | 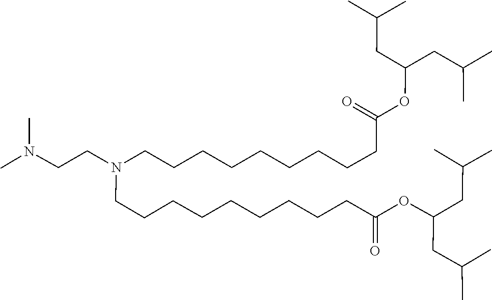 [SMILES: CC(C)CC(CC(C)C)OC(=O)CCCCCCCCCN(CCCCCCCCCC(=O)OC(CC(C)C)CC(C)C)CCN(C)C] | A |
| I-9 | 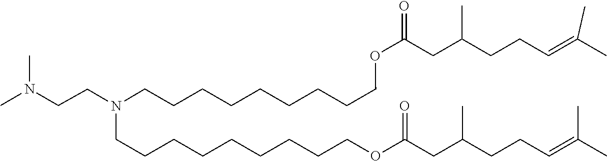 [SMILES: CC(C)=CCCC(C)CC(=O)OCCCCCCCCCN(CCCCCCCCCOC(=O)CC(C)CCC=C(C)C)CCN(C)C] | B |
| I-10 | 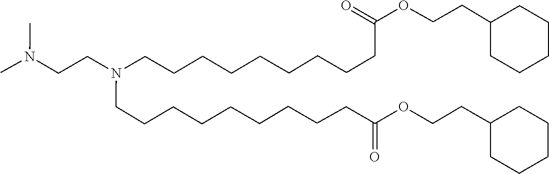 [SMILES: CN(C)CCN(CCCCCCCCCC(=O)OCCC1CCCCC1)CCCCCCCCCC(=O)OCCC1CCCCC1] | A |
| I-11 | 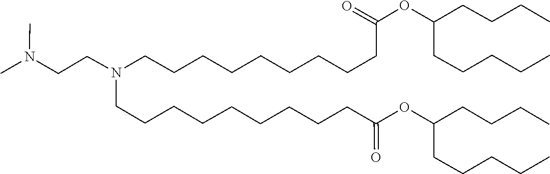 [SMILES: CCCCCC(CCCC)OC(=O)CCCCCCCCCN(CCCCCCCCCC(=O)OC(CCCC)CCCCC)CCN(C)C] | A |
| I-12 | 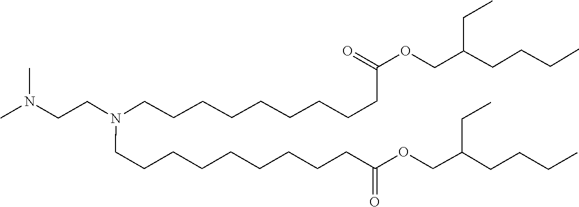 [SMILES: CCCCC(CC)COC(=O)CCCCCCCCCN(CCCCCCCCCC(=O)OCC(CC)CCCC)CCN(C)C] | A |
| I-13 | 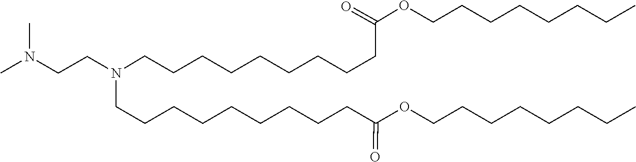 [SMILES: CCCCCCCCOC(=O)CCCCCCCCCN(CCCCCCCCCC(=O)OCCCCCCCC)CCN(C)C] | A |
| I-14 | 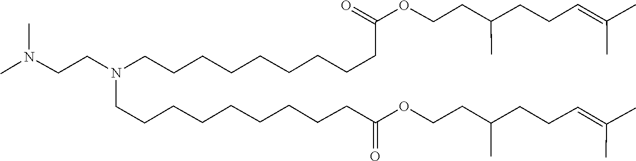 [SMILES: CC(C)=CCCC(C)CCOC(=O)CCCCCCCCCN(CCCCCCCCCC(=O)OCCC(C)CCC=C(C)C)CCN(C)C] | A |
| I-15 | 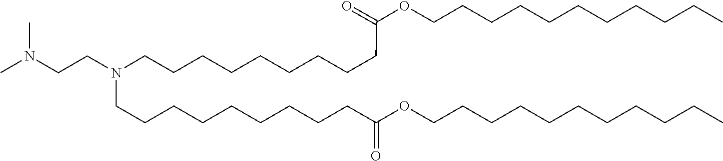 [SMILES: CCCCCCCCCCCOC(=O)CCCCCCCCCN(CCCCCCCCCC(=O)OCCCCCCCCCCC)CCN(C)C] | A |
| I-16 | 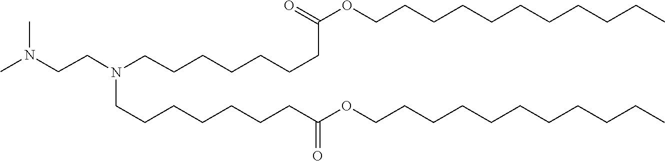 [SMILES: CCCCCCCCCCCOC(=O)CCCCCCCN(CCCCCCCC(=O)OCCCCCCCCCCC)CCN(C)C] | A |
| I-17 | 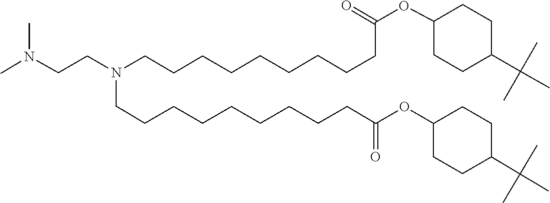 [SMILES: CN(C)CCN(CCCCCCCCCC(=O)OC1CCC(C(C)(C)C)CC1)CCCCCCCCCC(=O)OC1CCC(C(C)(C)C)CC1] | A |
| I-18 | 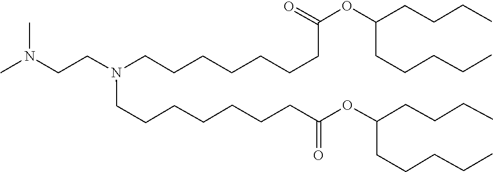 [SMILES: CCCCCC(CCCC)OC(=O)CCCCCCCN(CCCCCCCC(=O)OC(CCCC)CCCCC)CCN(C)C] | A |
| I-19 | 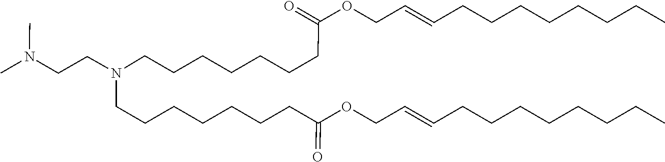 [SMILES: CCCCCCCC/C=C/COC(=O)CCCCCCCN(CCCCCCCC(=O)OC/C=C/CCCCCCCC)CCN(C)C] | A |
| I-20 | 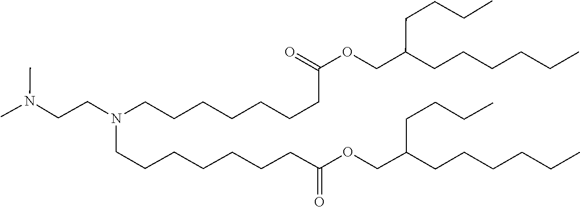 [SMILES: CCCCCCC(CCCC)COC(=O)CCCCCCCN(CCCCCCCC(=O)OCC(CCCC)CCCCCC)CCN(C)C] | A |
| I-21 | 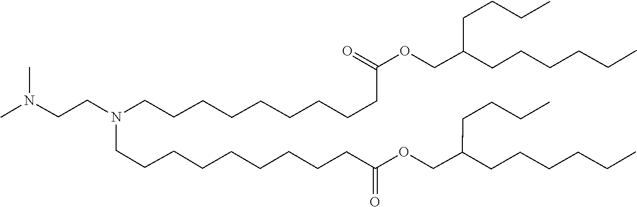 [SMILES: CCCCCCC(CCCC)COC(=O)CCCCCCCCCN(CCCCCCCCCC(=O)OCC(CCCC)CCCCCC)CCN(C)C] | A |
| I-22 | 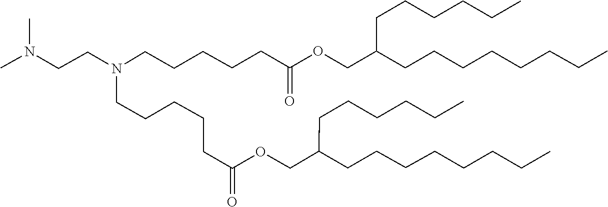 [SMILES: CCCCCCCCC(CCCCCC)COC(=O)CCCCCN(CCCCCC(=O)OCC(CCCCCC)CCCCCCCC)CCN(C)C] | A |
| I-23 | 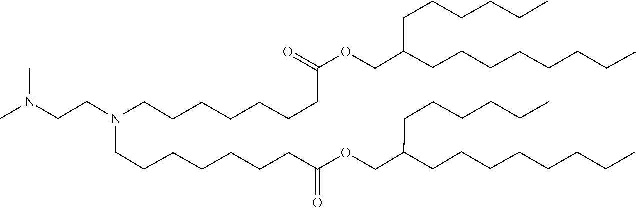 [SMILES: CCCCCCCCC(CCCCCC)COC(=O)CCCCCCCN(CCCCCCCC(=O)OCC(CCCCCC)CCCCCCCC)CCN(C)C] | A |
| I-24 | 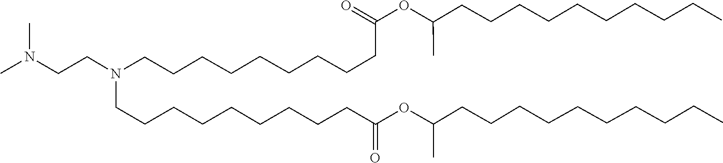 [SMILES: CCCCCCCCCCC(C)OC(=O)CCCCCCCCCN(CCCCCCCCCC(=O)OC(C)CCCCCCCCCC)CCN(C)C] | A |
| I-25 | 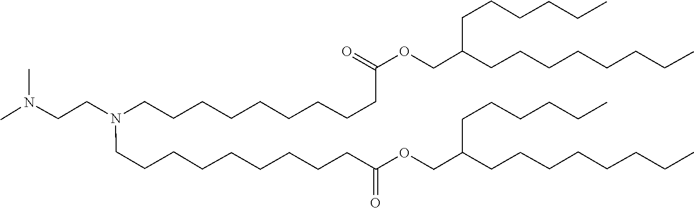 [SMILES: CCCCCCCCC(CCCCCC)COC(=O)CCCCCCCCCN(CCCCCCCCCC(=O)OCC(CCCCCC)CCCCCCCC)CCN(C)C] | A |
| I-26 | 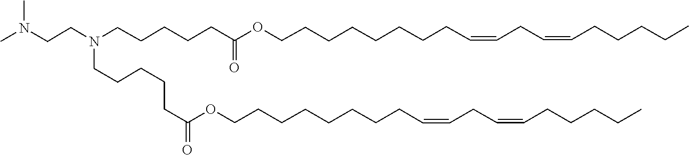 [SMILES: CCCCC/C=C\C/C=C\CCCCCCCCOC(=O)CCCCCN(CCCCCC(=O)OCCCCCCCC/C=C\C/C=C\CCCCC)CCN(C)C] | A |
| I-27 | 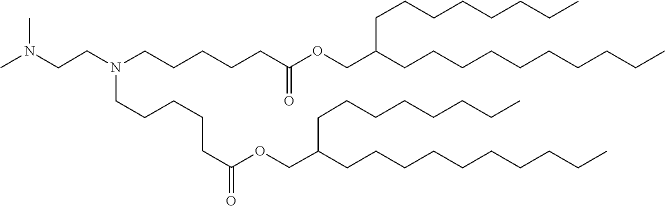 [SMILES: CCCCCCCCCCC(CCCCCCCC)COC(=O)CCCCCN(CCCCCC(=O)OCC(CCCCCCCC)CCCCCCCCCC)CCN(C)C] | A |
| I-28 | 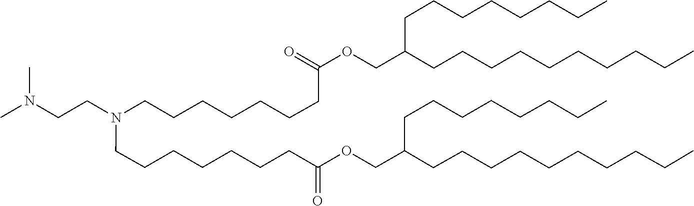 [SMILES: CCCCCCCCCCC(CCCCCCCC)COC(=O)CCCCCCCN(CCCCCCCC(=O)OCC(CCCCCCCC)CCCCCCCCCC)CCN(C)C] | A |
| I-29 | 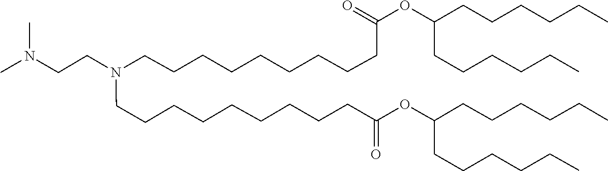 [SMILES: CCCCCCC(CCCCCC)OC(=O)CCCCCCCCCN(CCCCCCCCCC(=O)OC(CCCCCC)CCCCCC)CCN(C)C] | A |
| I-30 | 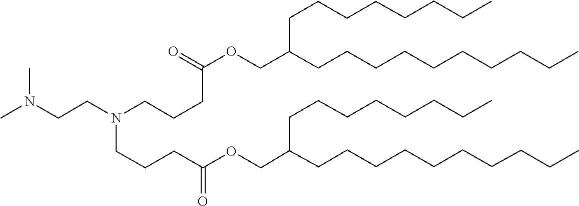 [SMILES: CCCCCCCCCCC(CCCCCCCC)COC(=O)CCCN(CCCC(=O)OCC(CCCCCCCC)CCCCCCCCCC)CCN(C)C] | A |
| I-31 | 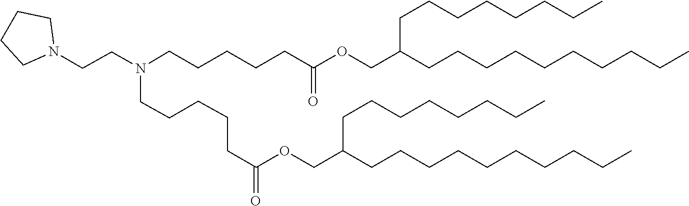 [SMILES: CCCCCCCCCCC(CCCCCCCC)COC(=O)CCCCCN(CCCCCC(=O)OCC(CCCCCCCC)CCCCCCCCCC)CCN1CCCC1] | C |
| I-32 | 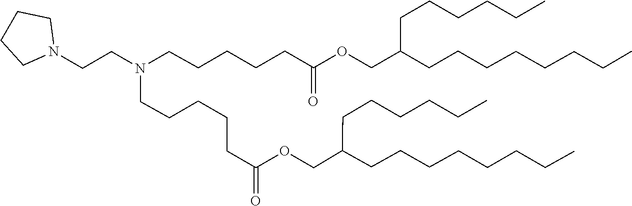 [SMILES: CCCCCCCCC(CCCCCC)COC(=O)CCCCCN(CCCCCC(=O)OCC(CCCCCC)CCCCCCCC)CCN1CCCC1] | |
| I-33 | 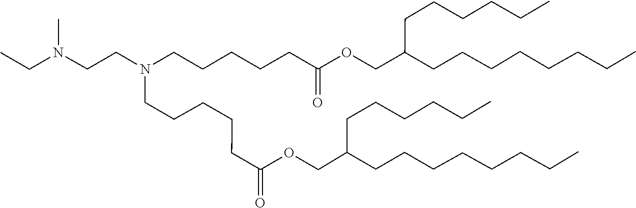 [SMILES: CCCCCCCCC(CCCCCC)COC(=O)CCCCCN(CCCCCC(=O)OCC(CCCCCC)CCCCCCCC)CCN(C)CC] | C |
| I-34 | 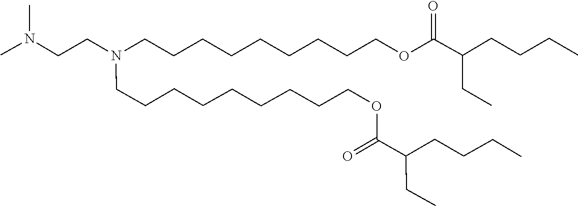 [SMILES: CCCCC(CC)C(=O)OCCCCCCCCCN(CCCCCCCCCOC(=O)C(CC)CCCC)CCN(C)C] | B |
| I-35 | 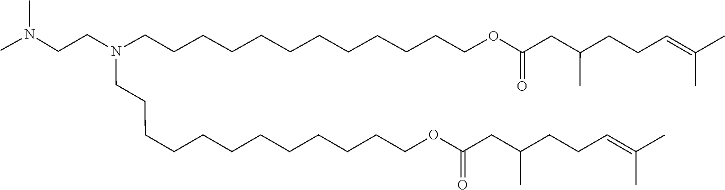 [SMILES: CC(C)=CCCC(C)CC(=O)OCCCCCCCCCCCCN(CCCCCCCCCCCCOC(=O)CC(C)CCC=C(C)C)CCN(C)C] | B |
| I-36 | 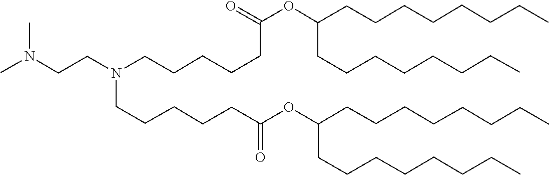 [SMILES: CCCCCCCCC(CCCCCCCC)OC(=O)CCCCCN(CCCCCC(=O)OC(CCCCCCCC)CCCCCCCC)CCN(C)C] | C |
| I-37 | 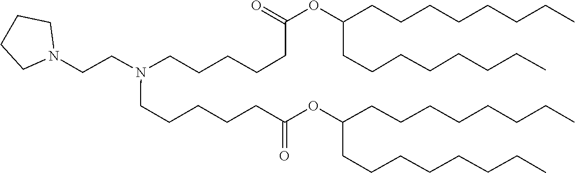 [SMILES: CCCCCCCCC(CCCCCCCC)OC(=O)CCCCCN(CCCCCC(=O)OC(CCCCCCCC)CCCCCCCC)CCN1CCCC1] | C |
| I-38 | 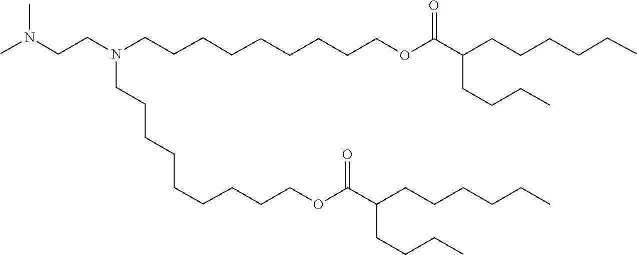 [SMILES: CCCCCCC(CCCC)C(=O)OCCCCCCCCCN(CCCCCCCCCOC(=O)C(CCCC)CCCCCC)CCN(C)C] | B |
| I-39 | 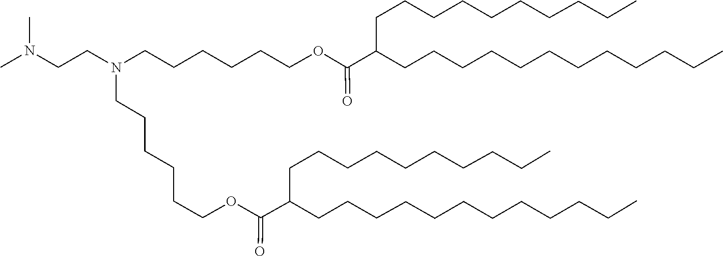 [SMILES: CCCCCCCCCCCCC(CCCCCCCCCC)C(=O)OCCCCCCN(CCCCCCOC(=O)C(CCCCCCCCCC)CCCCCCCCCCCC)CCN(C)C] | B |
| I-40 | 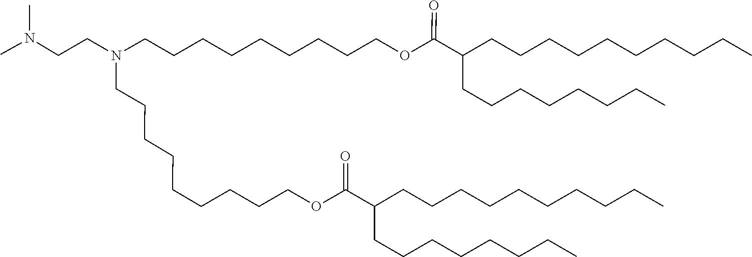 [SMILES: CCCCCCCCCCC(CCCCCCCC)C(=O)OCCCCCCCCCN(CCCCCCCCCOC(=O)C(CCCCCCCC)CCCCCCCCCC)CCN(C)C] | B |
| I-41 | 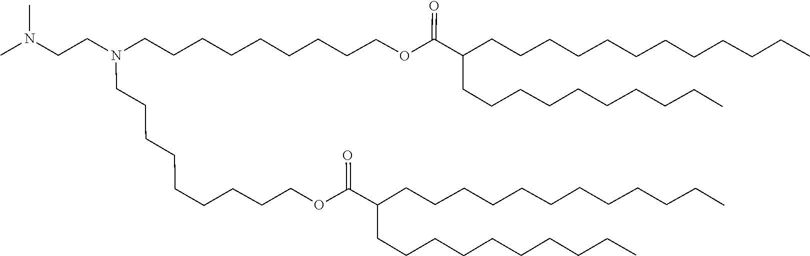 [SMILES: CCCCCCCCCCCCC(CCCCCCCCCC)C(=O)OCCCCCCCCCN(CCCCCCCCCOC(=O)C(CCCCCCCCCC)CCCCCCCCCCCC)CCN(C)C] | B |

In some embodiments, the LNPs comprise a lipid of Formula (I), a nucleoside-modified RNA and one or more excipients selected from neutral lipids, steroids and pegylated lipids. In some embodiments the lipid of Formula (I) is compound 1-5. In some embodiments the lipid of Formula (I) is compound 1-6.

In some other embodiments, the cationic lipid component of the LNPs has the structure of Formula (II):

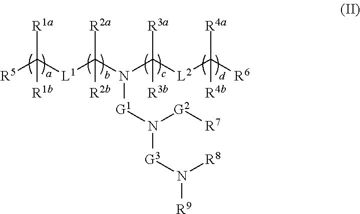

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}, —OC(═O)NR^{a}—, —NR^{a}C(═O)O—, or a direct bond;
G¹ is C1-C2 alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a} or a direct bond;
G³ is C1-C6 alkylene;
R^{a} is H or C1-C12 alkyl;
R^{1a} and R^{1b} are, at each occurrence, independently either: (a) H or C1-C12 alkyl; or (b) R^{1a} is H or C1-C12 alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C1-C12 alkyl; or (b) R^{2a} is H or C1-C12 alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either: (a) H or C1-C12 alkyl; or (b) R^{3a} is H or C1-C12 alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C1-C12 alkyl; or (b) R^{4a} is H or C1-C12 alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁵ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In some embodiments of Formula (II), L¹ and L² are each independently —O(C═O)—, —(C═O)O— or a direct bond. In other embodiments, G¹ and G² are each independently —(C═O)— or a direct bond. In some different embodiments, L¹ and L² are each independently —O(C═O)—, —(C═O)O— or a direct bond; and G¹ and G² are each independently —(C═O)— or a direct bond.

In some different embodiments of Formula (II), L¹ and L² are each independently —C(═O)—, —O—, —S(O)ₓ—, —S—S—, —C(═O)S—, —SC(═O)—, —NR^{a}—, —NR^{a}C(═O)—, —C(═O)NR^{a}—, —NR^{a}C(═O)NR^{a}, —OC(═O)NR^{a}—, —NR^{a}C(═O)O—, —NR^{a}S(O)ₓNR^{a}—, —NR^{a}S(O)ₓ— or —S(O)ₓNR^{a}—.

In other of the foregoing embodiments of Formula (II), the lipid compound has one of the following structures (IIA) or (IIB):

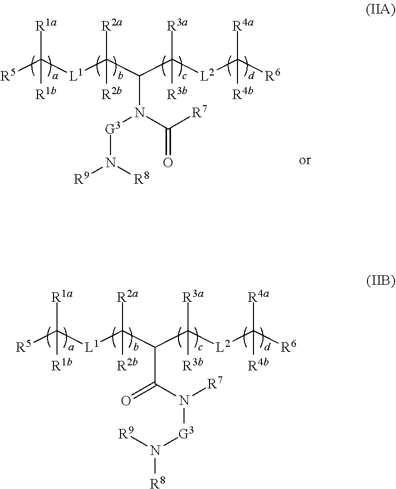

In some embodiments of Formula (II), the lipid compound has structure (IIA). In other embodiments, the lipid compound has structure (IIB).

In any of the foregoing embodiments of Formula (II), one of L1 or L2 is —O(C═O)—. For example, in some embodiments each of L1 and L2 are —O(C═O)—.

In some different embodiments of Formula (II), one of L1 or L2 is —(C═O)O—. For example, in some embodiments each of L1 and L2 is —(C═O)O—.

In different embodiments of Formula (II), one of L1 or L2 is a direct bond. As used herein, a “direct bond” means the group (e.g., L1 or L2) is absent. For example, in some embodiments each of L1 and L2 is a direct bond.

In other different embodiments of Formula (II), for at least one occurrence of R^{1a} and R^{1b}, R^{1a} is H or C1-C12 alkyl, and R^{1b} together with the carbon atom to which it is bound is taken together with an adjacent R^{1b} and the carbon atom to which it is bound to form a carbon-carbon double bond.

In still other different embodiments of Formula (II), for at least one occurrence of R4a and R4b, R4a is H or C1-C12 alkyl, and R4b together with the carbon atom to which it is bound is taken together with an adjacent R4b and the carbon atom to which it is bound to form a carbon-carbon double bond.

In more embodiments of Formula (II), for at least one occurrence of R2a and R2b, R2a is H or C1-C12 alkyl, and R2b together with the carbon atom to which it is bound is taken together with an adjacent R2b and the carbon atom to which it is bound to form a carbon-carbon double bond.

In other different embodiments of Formula (II), for at least one occurrence of R3a and R3b, R3a is H or C1-C12 alkyl, and R3b together with the carbon atom to which it is bound is taken together with an adjacent R3b and the carbon atom to which it is bound to form a carbon-carbon double bond.

In various other embodiments of Formula (II), the lipid compound has one of the following structures (IIC) or (IID):

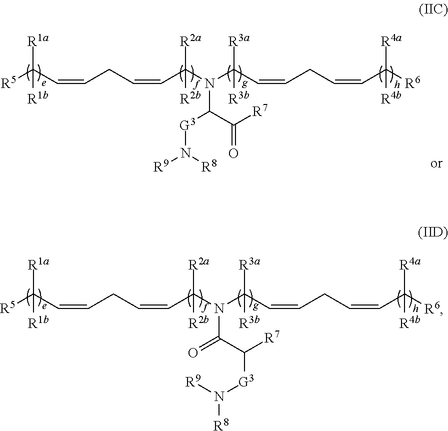

wherein e, f, g and h are each independently an integer from 1 to 12.

In some embodiments of Formula (II), the lipid compound has structure (IIC). In other embodiments, the lipid compound has structure (IID).

In various embodiments of structures (IIC) or (IID), e, f, g and h are each independently an integer from 4 to 10.

In some embodiments of Formula (II), a, b, c and d are each independently an integer from 2 to 12 or an integer from 4 to 12. In other embodiments, a, b, c and d are each independently an integer from 8 to 12 or 5 to 9. In some embodiments, a is 0. In some embodiments, a is 1. In other embodiments, a is 2. In more embodiments, a is 3. In yet other embodiments, a is 4. In some embodiments, a is 5. In other embodiments, a is 6. In more embodiments, a is 7. In yet other embodiments, a is 8. In some embodiments, a is 9. In other embodiments, a is 10. In more embodiments, a is 11. In yet other embodiments, a is 12. In some embodiments, a is 13. In other embodiments, a is 14. In more embodiments, a is 15. In yet other embodiments, a is 16.

In some embodiments of Formula (II), b is 1. In other embodiments, b is 2. In more embodiments, b is 3. In yet other embodiments, b is 4. In some embodiments, b is 5. In other embodiments, b is 6. In more embodiments, b is 7. In yet other embodiments, b is 8. In some embodiments, b is 9. In other embodiments, b is 10. In more embodiments, b is 11. In yet other embodiments, b is 12. In some embodiments, b is 13. In other embodiments, b is 14. In more embodiments, b is 15. In yet other embodiments, b is 16.

In some embodiments of Formula (II), c is 1. In other embodiments, c is 2. In more embodiments, c is 3. In yet other embodiments, c is 4. In some embodiments, c is 5. In other embodiments, c is 6. In more embodiments, c is 7. In yet other embodiments, c is 8. In some embodiments, c is 9. In other embodiments, c is 10. In more embodiments, c is 11. In yet other embodiments, c is 12. In some embodiments, c is 13. In other embodiments, c is 14. In more embodiments, c is 15. In yet other embodiments, c is 16.

In some embodiments of Formula (II), d is 0. In some embodiments, d is 1. In other embodiments, d is 2. In more embodiments, d is 3. In yet other embodiments, d is 4. In some embodiments, d is 5. In other embodiments, d is 6. In more embodiments, d is 7. In yet other embodiments, d is 8. In some embodiments, d is 9. In other embodiments, d is 10. In more embodiments, d is 11. In yet other embodiments, d is 12. In some embodiments, d is 13. In other embodiments, d is 14. In more embodiments, d is 15. In yet other embodiments, d is 16.

In some embodiments of Formula (II), e is 1. In other embodiments, e is 2. In more embodiments, e is 3. In yet other embodiments, e is 4. In some embodiments, e is 5. In other embodiments, e is 6. In more embodiments, e is 7. In yet other embodiments, e is 8. In some embodiments, e is 9. In other embodiments, e is 10. In more embodiments, e is 11. In yet other embodiments, e is 12.

In some embodiments of Formula (II), f is 1. In other embodiments, f is 2. In more embodiments, f is 3. In yet other embodiments, f is 4. In some embodiments, f is 5. In other embodiments, f is 6. In more embodiments, f is 7. In yet other embodiments, f is 8. In some embodiments, f is 9. In other embodiments, f is 10. In more embodiments, f is 11. In yet other embodiments, f is 12.

In some embodiments of Formula (II), g is 1. In other embodiments, g is 2. In more embodiments, g is 3. In yet other embodiments, g is 4. In some embodiments, g is 5. In other embodiments, g is 6. In more embodiments, g is 7. In yet other embodiments, g is 8. In some embodiments, g is 9. In other embodiments, g is 10. In more embodiments, g is 11. In yet other embodiments, g is 12.

In some embodiments of Formula (II), h is 1. In other embodiments, e is 2. In more embodiments, h is 3. In yet other embodiments, h is 4. In some embodiments, e is 5. In other embodiments, h is 6. In more embodiments, h is 7. In yet other embodiments, h is 8. In some embodiments, h is 9. In other embodiments, h is 10. In more embodiments, h is 11. In yet other embodiments, h is 12.

In some other various embodiments of Formula (II), a and d are the same. In some other embodiments, b and c are the same. In some other specific embodiments and a and d are the same and b and c are the same.

The sum of a and b and the sum of c and d of Formula (II) are factors which may be varied to obtain a lipid having the desired properties. In one embodiment, a and b are chosen such that their sum is an integer ranging from 14 to 24. In other embodiments, c and d are chosen such that their sum is an integer ranging from 14 to 24. In further embodiment, the sum of a and b and the sum of c and d are the same. For example, in some embodiments the sum of a and b and the sum of c and d are both the same integer which may range from 14 to 24. In still more embodiments, a, b, c and d are selected such that the sum of a and b and the sum of c and d is 12 or greater.

The substituents at R1a, R2a, R3a and R4a of Formula (II) are not particularly limited. In some embodiments, at least one of R1a, R2a, R3a and R4a is H. In some embodiments R1a, R2a, R3a and R4a are H at each occurrence. In some other embodiments at least one of R1a, R2a, R3a and R4a is C1-C12 alkyl. In some other embodiments at least one of R1a, R2a, R3a and R4a is C1-C8 alkyl. In some other embodiments at least one of R1a, R2a, R3a and R4a is C1-C6 alkyl. In some of the foregoing embodiments, the C1-C8 alkyl is methyl, ethyl, n-propyl, iso-propyl, n-butyl, iso-butyl, tert-butyl, n-hexyl or n-octyl.

In some embodiments of Formula (II), R1a, R1b, R4a and R4b are C1-C12 alkyl at each occurrence.

In further embodiments of Formula (II), at least one of R1b, R2b, R3b and R4b is H or R1b, R2b, R3b and R4b are H at each occurrence.

In some embodiments of Formula (II), R1b together with the carbon atom to which it is bound is taken together with an adjacent R1b and the carbon atom to which it is bound to form a carbon-carbon double bond. In other embodiments of the foregoing R4b together with the carbon atom to which it is bound is taken together with an adjacent R4b and the carbon atom to which it is bound to form a carbon-carbon double bond.

The substituents at R5 and R6 of Formula (II) are not particularly limited in the foregoing embodiments. In some embodiments one of R5 or R6 is methyl. In other embodiments each of R5 or R6 is methyl.

The substituents at R7 of Formula (II) are not particularly limited in the foregoing embodiments. In some embodiments R7 is C6-C16 alkyl. In some other embodiments, R7 is C6-C9 alkyl. In some of these embodiments, R7 is substituted with —(C═O)ORb, —O(C═O)Rb, —C(═O)Rb, —ORb, —S(O)xRb, —S—SRb, —C(═O)SRb, [0201]—SC(═O)Rb, —NRaRb, —NRaC(═O)Rb, —C(═O)NRaRb, —NRaC(═O)NRaRb, [0202]—OC(═O)NRaRb, —NRaC(═O)ORb, —NRaS(O)xNRaRb, —NRaS(O)xRb or —S(O)xNRaRb, wherein: Ra is H or C1-C12 alkyl; Rb is C1-C15 alkyl; and x is 0, 1 or 2. For example, in some embodiments R7 is substituted with —(C═O)ORb or —O(C═O)Rb.

In various of the foregoing embodiments of Formula (II), Rb is branched C1-C15 alkyl. For example, in some embodiments R^{b} has one of the following structures:

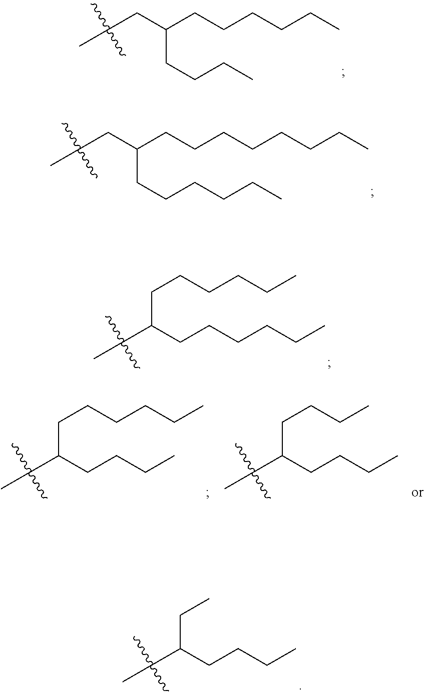

In some other of the foregoing embodiments of Formula (II), one of R8 or R9 is methyl. In other embodiments, both R8 and R9 are methyl.

In some different embodiments of Formula (II), R8 and R9, together with the nitrogen atom to which they are attached, form a 5, 6 or 7-membered heterocyclic ring. In some embodiments of the foregoing, R⁵ and R⁹, together with the nitrogen atom to which they are attached, form a 5-membered heterocyclic ring, for example a pyrrolidinyl ring. In some different embodiments of the foregoing, R⁵ and R⁹, together with the nitrogen atom to which they are attached, form a 6-membered heterocyclic ring, for example a piperazinyl ring.

In still other embodiments of the foregoing lipids of Formula (II), G3 is C2-C4 alkylene, for example C3 alkylene.

In various different embodiments, the lipid compound has one of the structures set forth in Table 2 below.

TABLE 2
Representative Lipids of Formula (II)

| | | Prep. |
|---|---|---|
| No. | Structure | Method |
| II-1 | 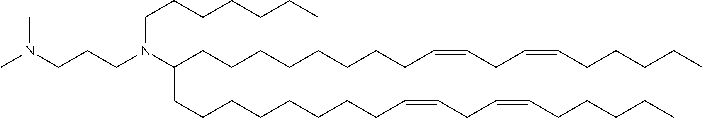 [SMILES: CCCCC/C=C\C/C=C\CCCCCCCCC(CCCCCCCC/C=C\C/C=C\CCCCC)N(CCCCCCC)CCCN(C)C] | D |
| II-2 | 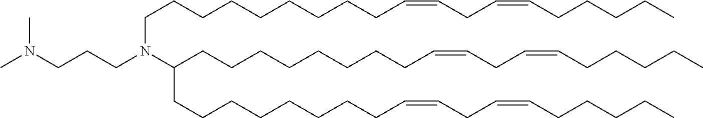 [SMILES: CCCCC/C=C\C/C=C\CCCCCCCCCN(CCCN(C)C)C(CCCCCCCC/C=C\C/C=C\CCCCC)CCCCCCCC/C=C\C/C=C\CCCCC] | D |
| II-3 | 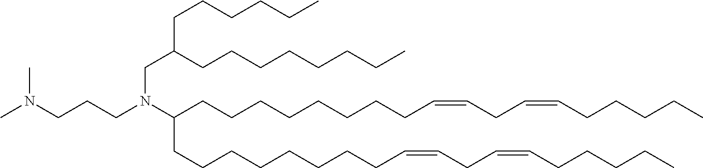 [SMILES: CCCCC/C=C\C/C=C\CCCCCCCCC(CCCCCCCC/C=C\C/C=C\CCCCC)N(CCCN(C)C)CC(CCCCCC)CCCCCCCC] | D |
| II-4 | 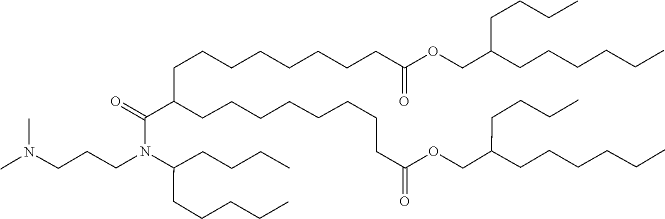 [SMILES: CCCCCCC(CCCC)COC(=O)CCCCCCCCC(CCCCCCCCC(=O)OCC(CCCC)CCCCCC)C(=O)N(CCCN(C)C)C(CCCC)CCCCC] | E |
| II-5 | 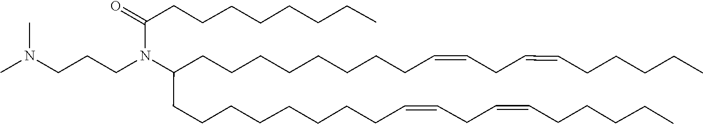 [SMILES: CCCCC/C=C\C/C=C\CCCCCCCCC(CCCCCCCC/C=C\C/C=C\CCCCC)N(CCCN(C)C)C(=O)CCCCCCCC] | D |
| II-6 | 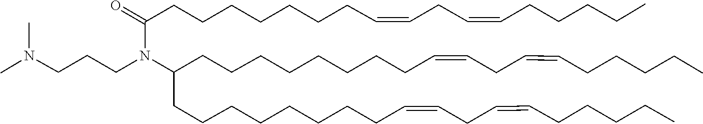 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC] | D |
| II-7 | 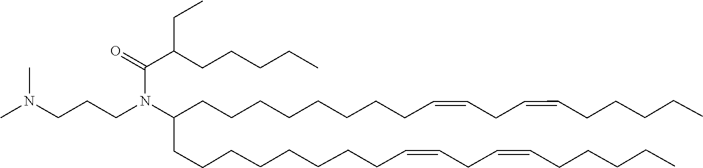 [SMILES: CCCCC/C=C\C/C=C\CCCCCCCCC(CCCCCCCC/C=C\C/C=C\CCCCC)N(CCCN(C)C)C(=O)C(CC)CCCCC] | D |
| II-8 | 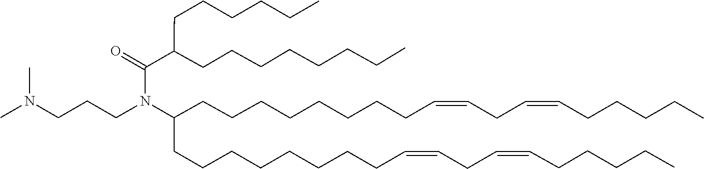 [SMILES: CCCCC/C=C\C/C=C\CCCCCCCCC(CCCCCCCC/C=C\C/C=C\CCCCC)N(CCCN(C)C)C(=O)C(CCCCCC)CCCCCCCC] | D |
| II-9 | 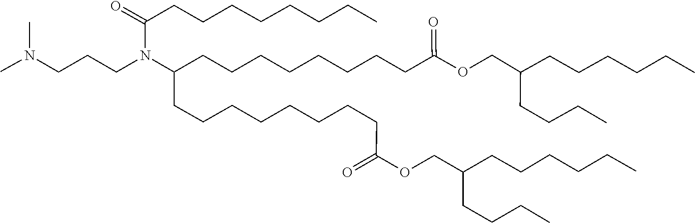 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] | D |
| II-10 | 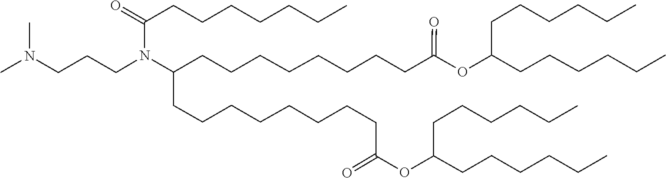 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-11 | 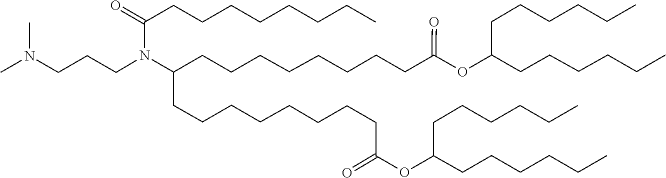 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] | D |
| II-12 | 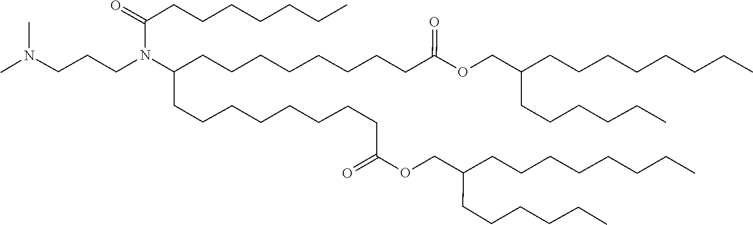 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] | D |
| II-13 | 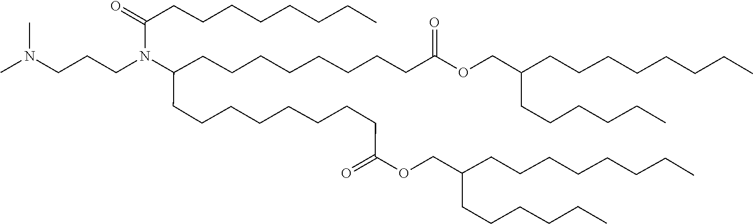 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] | D |
| II-14 | 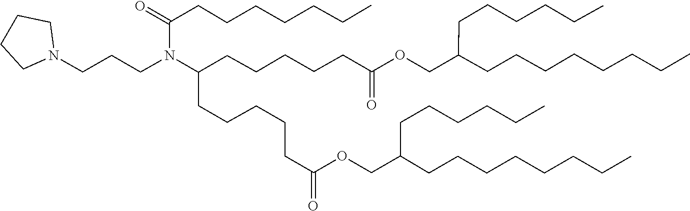 [SMILES: CCCCCCCCC(CCCCCC)COC(=O)CCCCCC(CCCCCC(=O)OCC(CCCCCC)CCCCCCCC)N(CCCN1CCCC1)C(=O)CCCCCCC] | D |
| II-15 | 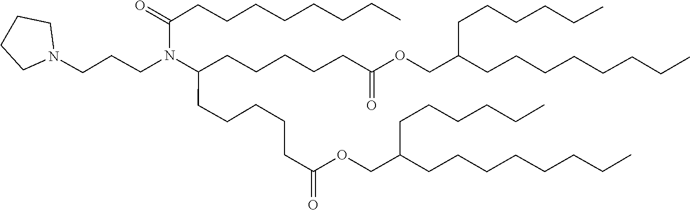 [SMILES: CCCCCCCCC(=O)N(CCCN1CCCC1)C(CCCCCC(=O)OCC(CCCCCC)CCCCCCCC)CCCCCC(=O)OCC(CCCCCC)CCCCCCCC] | A |
| II-16 | 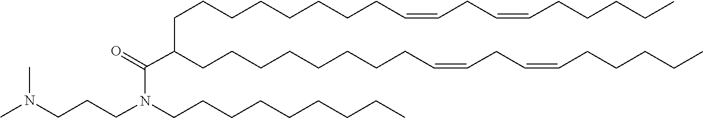 [SMILES: CCCCC/C=C\C/C=C\CCCCCCCCC(CCCCCCCC/C=C\C/C=C\CCCCC)C(=O)N(CCCCCCCCC)CCCN(C)C] | E |
| II-17 | 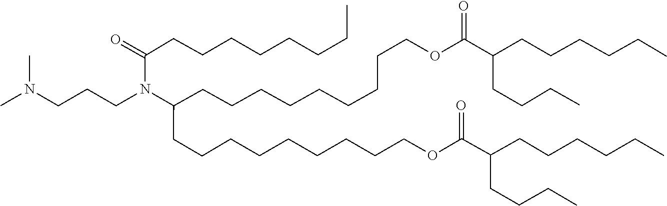 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] | A |
| II-18 | 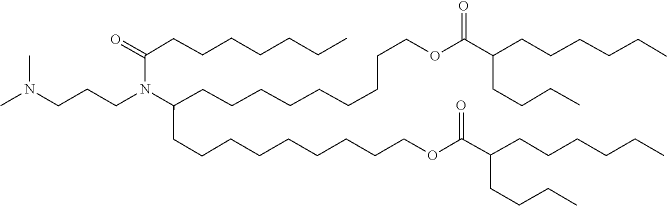 [SMILES: CCCCCCCC(=O)N(CCCN(C)C)C(CCCCCCCCCOC(=O)C(CCCC)CCCCCC)CCCCCCCCCOC(=O)C(CCCC)CCCCCC] | D |
| II-19 | 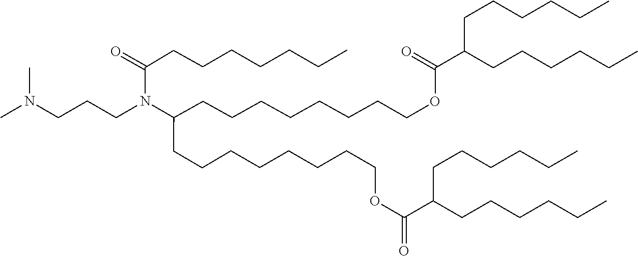 [SMILES: CCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-20 | 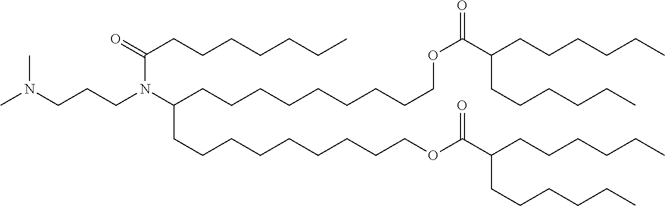 [SMILES: CCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-21 | 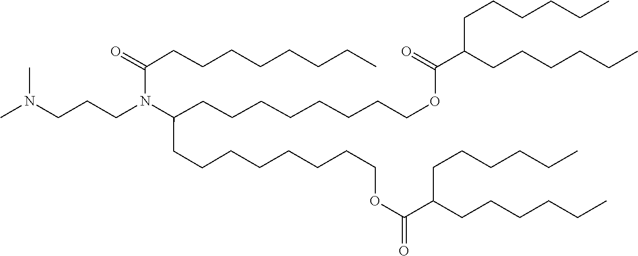 [SMILES: CCCCCCCCC(=O)N(CCCN(C)C)C(CCCCCCCCOC(=O)C(CCCCCC)CCCCCC)CCCCCCCCOC(=O)C(CCCCCC)CCCCCC] | D |
| II-22 | 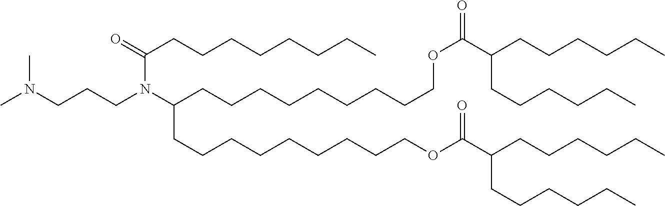 [SMILES: CCCCCCCCC(=O)N(CCCN(C)C)C(CCCCCCCCCOC(=O)C(CCCCCC)CCCCCC)CCCCCCCCCOC(=O)C(CCCCCC)CCCCCC] | D |
| II-23 | 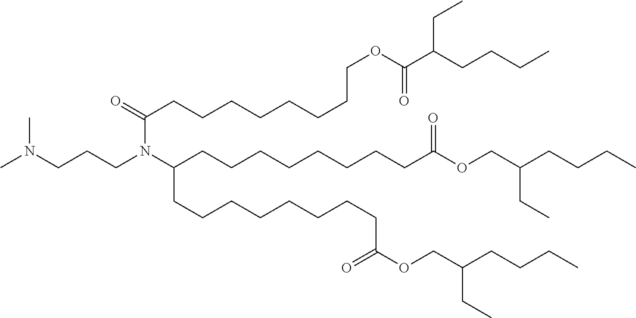 [SMILES: CCCCC(CC)COC(=O)CCCCCCCCC(CCCCCCCCC(=O)OCC(CC)CCCC)N(CCCN(C)C)C(=O)CCCCCCCCOC(=O)C(CC)CCCC] | D |
| II-24 | 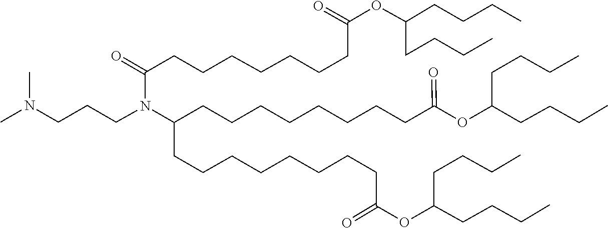 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] | D |
| II-25 | 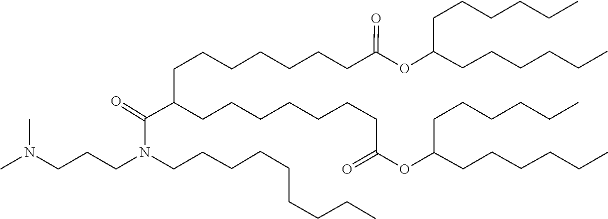 [SMILES: CCCCCCCCCN(CCCN(C)C)C(=O)C(CCCCCCCC(=O)OC(CCCCCC)CCCCCC)CCCCCCCC(=O)OC(CCCCCC)CCCCCC] | E |
| II-26 | 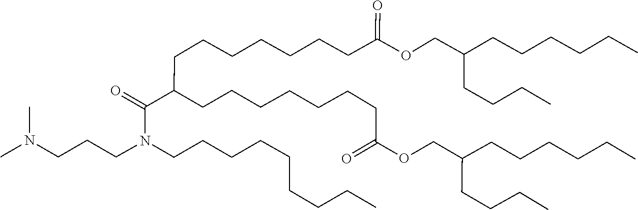 [SMILES: CCCCCCCCCN(CCCN(C)C)C(=O)C(CCCCCCCC(=O)OCC(CCCC)CCCCCC)CCCCCCCC(=O)OCC(CCCC)CCCCCC] | E |
| II-27 | 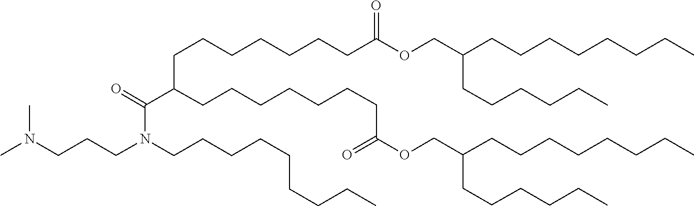 [SMILES: CCCCCCCCCN(CCCN(C)C)C(=O)C(CCCCCCCC(=O)OCC(CCCCCC)CCCCCCCC)CCCCCCCC(=O)OCC(CCCCCC)CCCCCCCC] | E |
| II-28 | 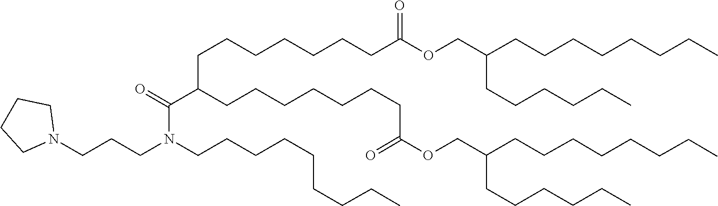 [SMILES: CCCCCCCCCN(CCCN1CCCC1)C(=O)C(CCCCCCCC(=O)OCC(CCCCCC)CCCCCCCC)CCCCCCCC(=O)OCC(CCCCCC)CCCCCCCC] | E |
| II-29 | 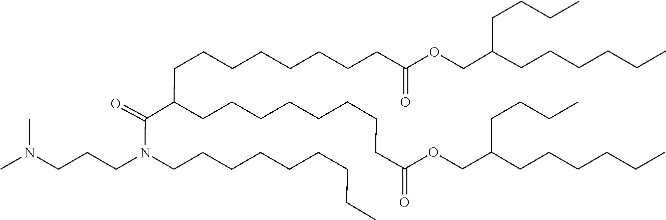 [SMILES: CCCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-30 | 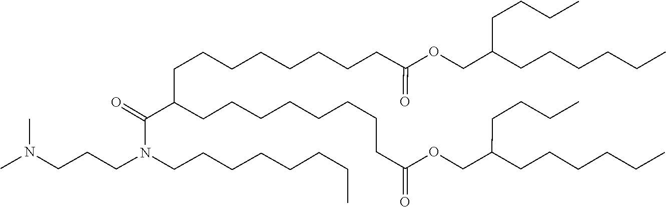 [SMILES: CCCCCCCCN(CCCN(C)C)C(=O)C(CCCCCCCCC(=O)OCC(CCCC)CCCCCC)CCCCCCCCC(=O)OCC(CCCC)CCCCCC] | E |
| II-31 | 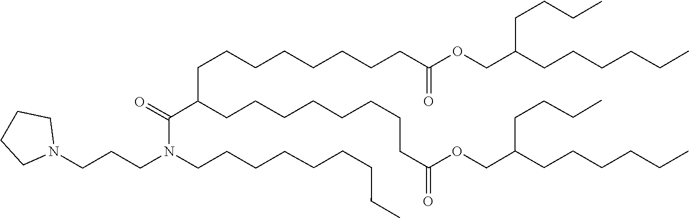 [SMILES: CCCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-32 | 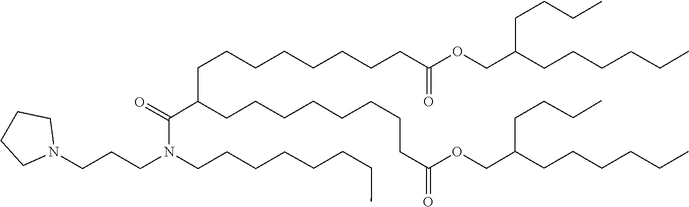 [SMILES: CCCCCCCCN(CCCN1CCCC1)C(=O)C(CCCCCCCCC(=O)OCC(CCCC)CCCCCC)CCCCCCCCC(=O)OCC(CCCC)CCCCCC] | E |
| II-33 | 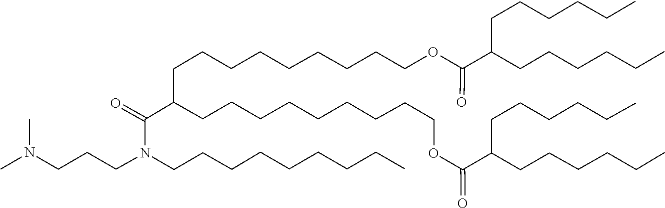 [SMILES: CCCCCCCCCN(CCCN(C)C)C(=O)C(CCCCCCCCCOC(=O)C(CCCCCC)CCCCCC)CCCCCCCCCOC(=O)C(CCCCCC)CCCCCC] | E |
| II-34 | 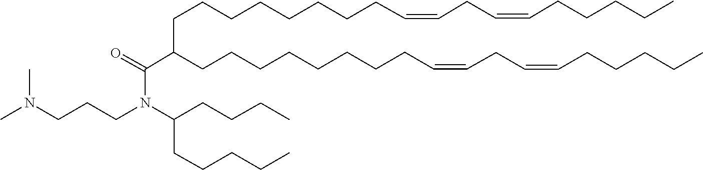 [SMILES: CCCCC/C=C\C/C=C\CCCCCCCCC(CCCCCCCC/C=C\C/C=C\CCCCC)C(=O)N(CCCN(C)C)C(CCCC)CCCCC] | E |
| II-35 | 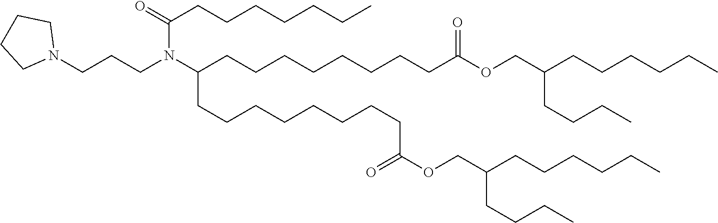 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] | |
| II-36 | 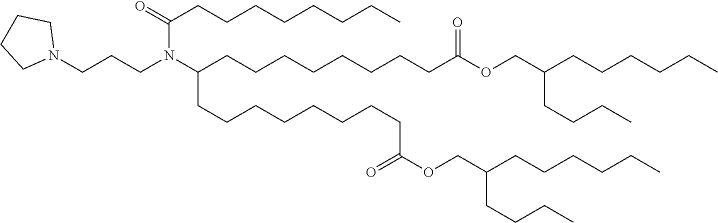 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] | D |

In some embodiments, the LNPs comprise a lipid of Formula (II), a nucleoside-modified RNA and one or more excipient selected from neutral lipids, steroids and pegylated lipids. In some embodiments the lipid of Formula (II) is compound II-9. In some embodiments the lipid of Formula (II) is compound II-10. In some embodiments the lipid of Formula (II) is compound II-11. In some embodiments the lipid of Formula (II) is compound II-12. In some embodiments the lipid of Formula (II) is compound II-32.

In some other embodiments, the cationic lipid component of the LNPs has the structure of Formula (III):

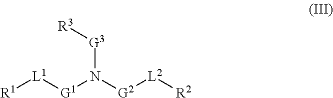

or a pharmaceutically acceptable salt, tautomer, prodrug or stereoisomer thereof, wherein.
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L or L2 is —O(C═O)—, —(C═O)—, —C(═O)—, —O—, —S(O)—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C1-C12 alkylene or C1-C12 alkenylene;
G³ is C1-C24 alkylene, C1-C24 alkenylene, C3-C8 cycloalkylene, C₃-C₈ cycloalkenylene;
R^{a} is H or C1-C12 alkyl;
R¹ and R² are each independently C6-C24 alkyl or C6-C24 alkenyl;
R³ is H, OR′, CN, —C(═O)OR⁴, —OC(═O)R⁴ or —NR⁵C(═O)R⁴;
R⁴ is C1-C12 alkyl;
R⁵ is H or C1-C6 alkyl; and
x is 0, 1 or 2.

In some of the foregoing embodiments of Formula (III), the lipid has one of the following structures (IIIA) or (IIIB):

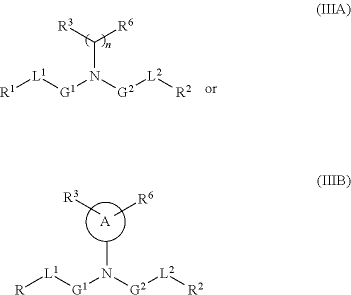

wherein:
A is a 3 to 8-membered cycloalkyl or cycloalkylene ring;
R⁶ is, at each occurrence, independently H, OH or C1-C24 alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

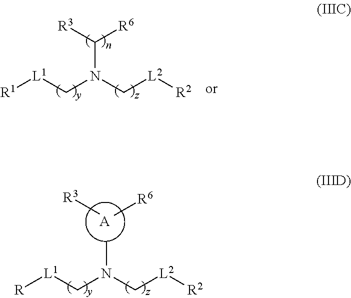

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

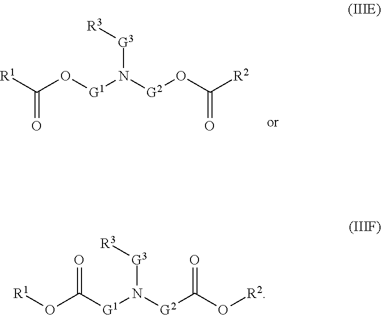

In some of the foregoing embodiments of Formula (III), the lipid has one of the following structures (IIIG), (IIIH), (IIII), or (IIIJ):

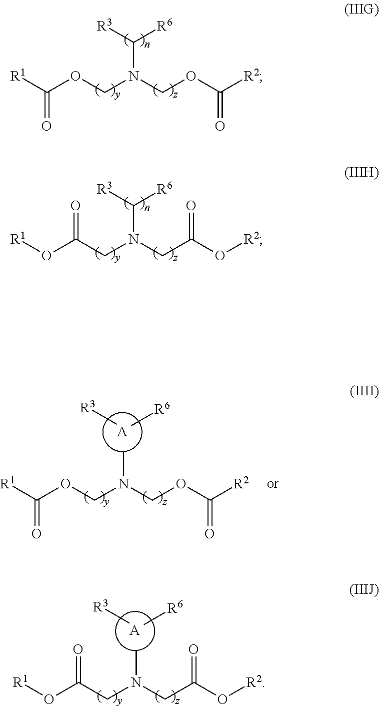

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R6 is H. In other of the foregoing embodiments, R6 is C1-C24 alkyl. In other embodiments, R6 is OH.

In some embodiments of Formula (III), G3 is unsubstituted. In other embodiments, G3 is substituted. In various different embodiments, G3 is linear C1-C24 alkylene or linear C1-C24 alkenylene.

In some other foregoing embodiments of Formula (III), R1 or R2, or both, is C6-C24 alkenyl. For example, in some embodiments, R¹ and R² each, independently have the following structure:

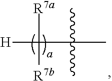

wherein:
R^{7a} and R^{7b} are, at each occurrence, independently H or C1-C12 alkyl; and
a is an integer from 2 to 12,
   wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R7a is H. For example, in some embodiments, R7a is H at each occurrence. In other different embodiments of the foregoing, at least one occurrence of R7b is C1-C8 alkyl. For example, in some embodiments, C1-C8 alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

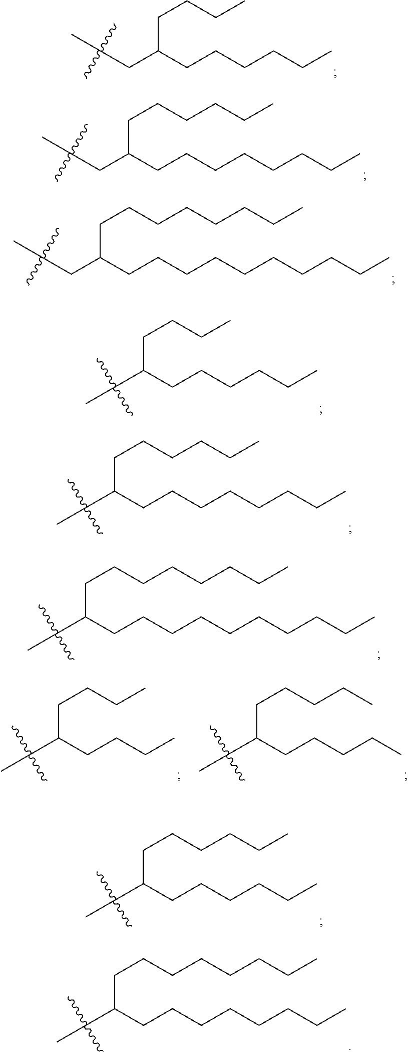

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In various different embodiments, the cationic lipid of Formula (III) has one of the structures set forth in Table 3 below.

TABLE 3
Representative Compounds of Formula (III)

| | | Prep. |
|---|---|---|
| No. | Structure | Method |
| III-1 | 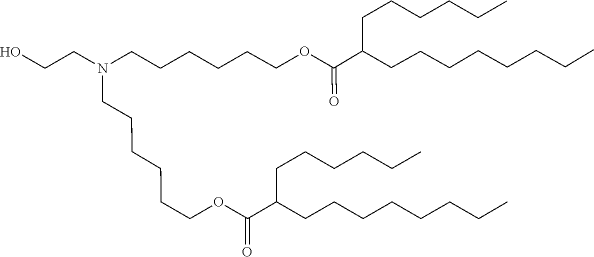 [SMILES: CCCCCCCCC(CCCCCC)C(=O)OCCCCCCN(CCO)CCCCCCOC(=O)C(CCCCCC)CCCCCCCC] | F |
| III-2 | 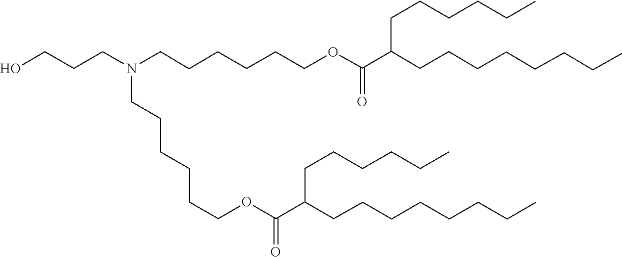 [SMILES: CCCCCCCCC(CCCCCC)C(=O)OCCCCCCN(CCCO)CCCCCCOC(=O)C(CCCCCC)CCCCCCCC] | F |
| III-3 | 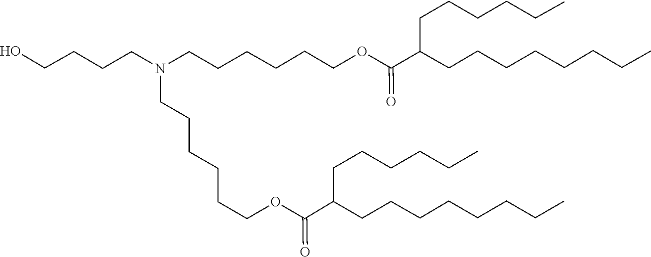 [SMILES: CCCCCCCCC(CCCCCC)C(=O)OCCCCCCN(CCCCO)CCCCCCOC(=O)C(CCCCCC)CCCCCCCC] | F |
| III-4 | 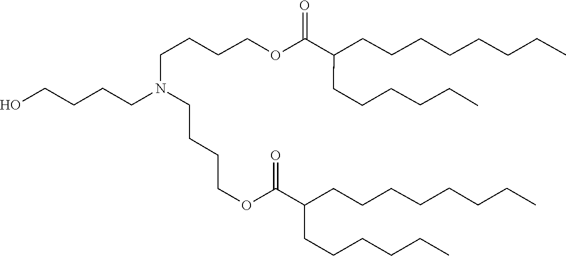 [SMILES: CCCCCCCCC(CCCCCC)C(=O)OCCCCN(CCCCO)CCCCOC(=O)C(CCCCCC)CCCCCCCC] | F |
| III-5 | 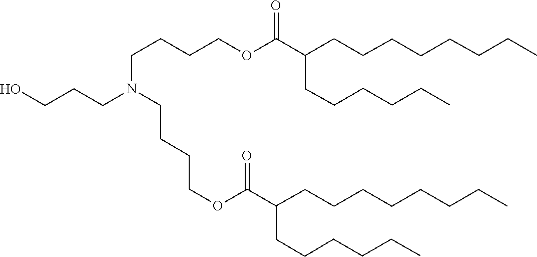 [SMILES: CCCCCCCCC(CCCCCC)C(=O)OCCCCN(CCCO)CCCCOC(=O)C(CCCCCC)CCCCCCCC] | F |
| III-6 | 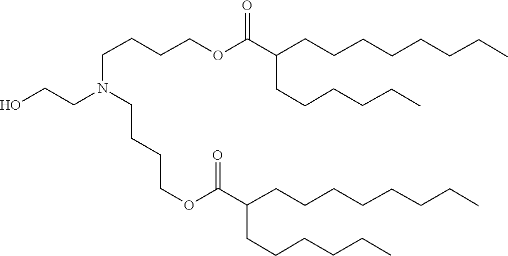 [SMILES: CCCCCCCCC(CCCCCC)C(=O)OCCCCN(CCO)CCCCOC(=O)C(CCCCCC)CCCCCCCC] | F |
| III-7 | 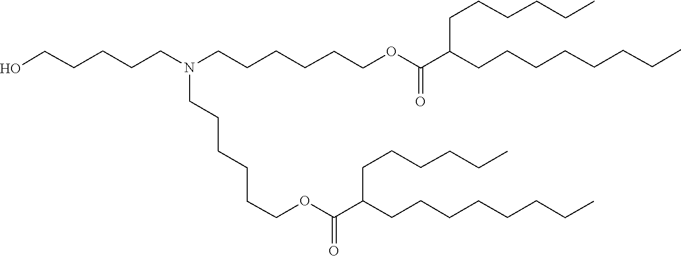 [SMILES: CCCCCCCCC(CCCCCC)C(=O)OCCCCCCN(CCCCCO)CCCCCCOC(=O)C(CCCCCC)CCCCCCCC] | F |
| III-8 | 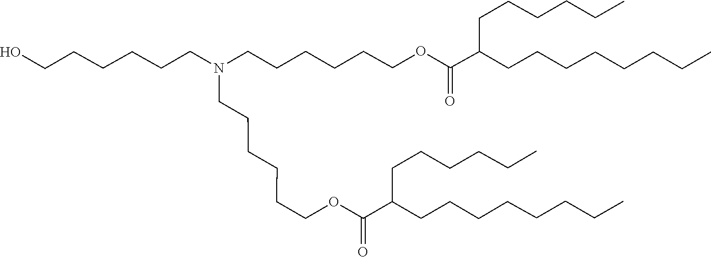 [SMILES: CCCCCCCCC(CCCCCC)C(=O)OCCCCCCN(CCCCCCO)CCCCCCOC(=O)C(CCCCCC)CCCCCCCC] | F |
| III-9 | 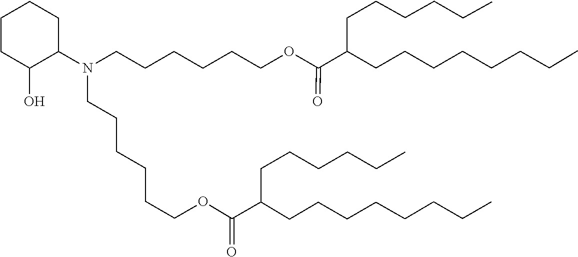 [SMILES: CCCCCCCCC(CCCCCC)C(=O)OCCCCCCN(CCCCCCOC(=O)C(CCCCCC)CCCCCCCC)C1CCCCC1O] | F |
| III-10 | 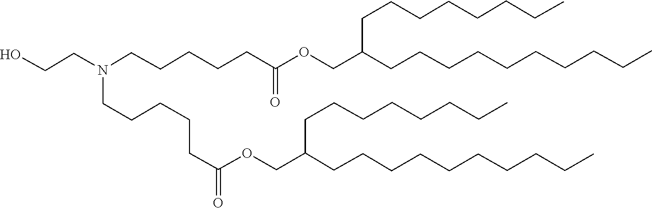 [SMILES: CCCCCCCCCCC(CCCCCCCC)COC(=O)CCCCCN(CCO)CCCCCC(=O)OCC(CCCCCCCC)CCCCCCCCCC] | F |
| III-11 | 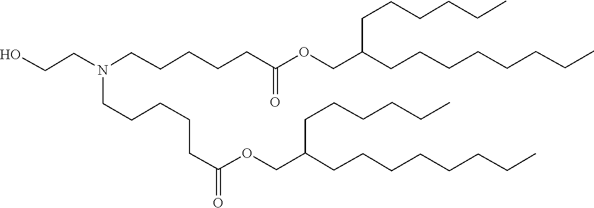 [SMILES: CCCCCCCCC(CCCCCC)COC(=O)CCCCCN(CCO)CCCCCC(=O)OCC(CCCCCC)CCCCCCCC] | F |
| III-12 | 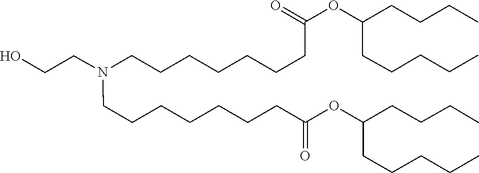 [SMILES: CCCCCC(CCCC)OC(=O)CCCCCCCN(CCO)CCCCCCCC(=O)OC(CCCC)CCCCC] | F |
| III-13 | 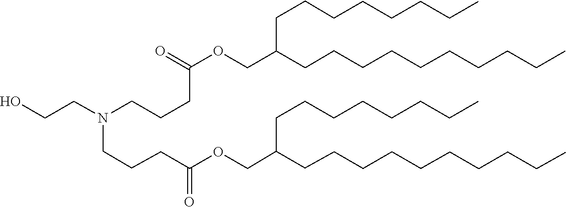 [SMILES: CCCCCCCCCCC(CCCCCCCC)COC(=O)CCCN(CCO)CCCC(=O)OCC(CCCCCCCC)CCCCCCCCCC] | F |
| III-14 | 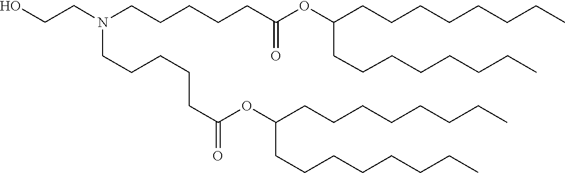 [SMILES: CCCCCCCCC(CCCCCCCC)OC(=O)CCCCCN(CCO)CCCCCC(=O)OC(CCCCCCCC)CCCCCCCC] | F |
| III-15 | 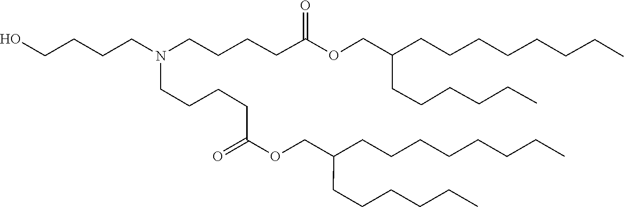 [SMILES: CCCCCCCCC(CCCCCC)COC(=O)CCCCN(CCCCO)CCCCC(=O)OCC(CCCCCC)CCCCCCCC] | F |
| III-16 | 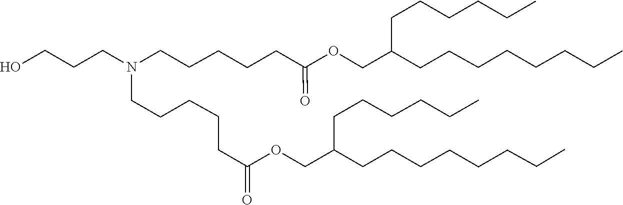 [SMILES: CCCCCCCCC(CCCCCC)COC(=O)CCCCCN(CCCO)CCCCCC(=O)OCC(CCCCCC)CCCCCCCC] | F |
| III-17 | 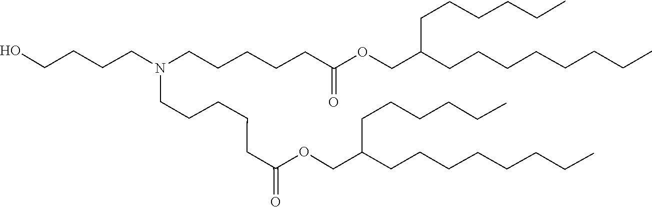 [SMILES: CCCCCCCCC(CCCCCC)COC(=O)CCCCCN(CCCCO)CCCCCC(=O)OCC(CCCCCC)CCCCCCCC] | F |
| III-18 | 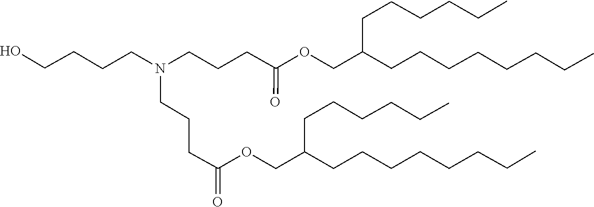 [SMILES: CCCCCCCCC(CCCCCC)COC(=O)CCCN(CCCCO)CCCC(=O)OCC(CCCCCC)CCCCCCCC] | F |
| III-19 | 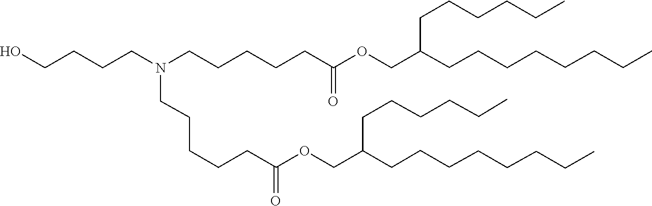 [SMILES: CCCCCCCCC(CCCCCC)COC(=O)CCCCCN(CCCCO)CCCCCC(=O)OCC(CCCCCC)CCCCCCCC] | F |
| III-20 | 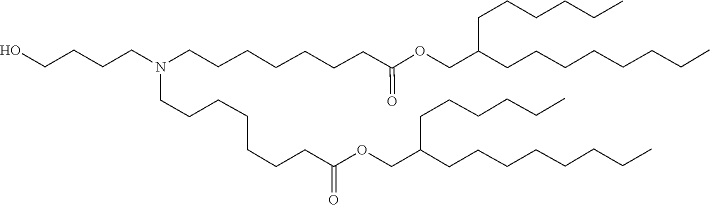 [SMILES: CCCCCCCCC(CCCCCC)COC(=O)CCCCCCCN(CCCCO)CCCCCCCC(=O)OCC(CCCCCC)CCCCCCCC] | F |
| III-21 | 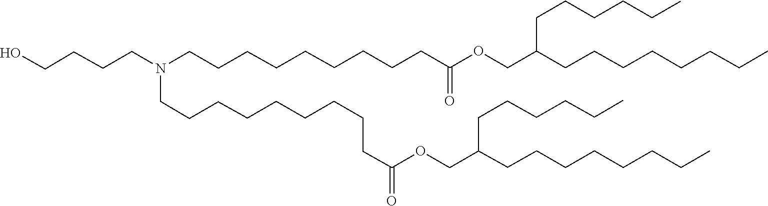 [SMILES: CCCCCCCCC(CCCCCC)COC(=O)CCCCCCCCCN(CCCCO)CCCCCCCCCC(=O)OCC(CCCCCC)CCCCCCCC] | F |
| III-22 | 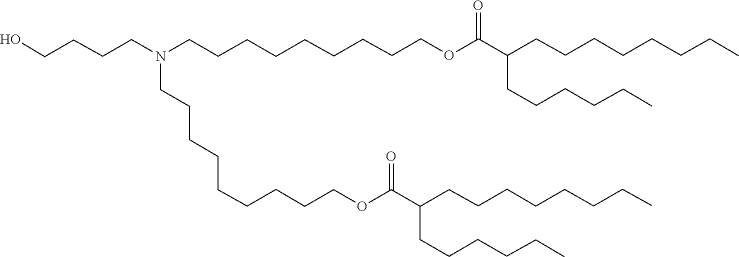 [SMILES: CCCCCCCCC(CCCCCC)C(=O)OCCCCCCCCCN(CCCCO)CCCCCCCCCOC(=O)C(CCCCCC)CCCCCCCC] | F |
| III-23 | 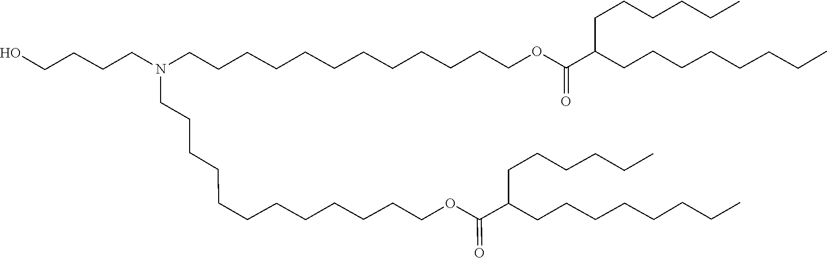 [SMILES: CCCCCCCCC(CCCCCC)C(=O)OCCCCCCCCCCCCN(CCCCO)CCCCCCCCCCCCOC(=O)C(CCCCCC)CCCCCCCC] | F |
| III-24 | 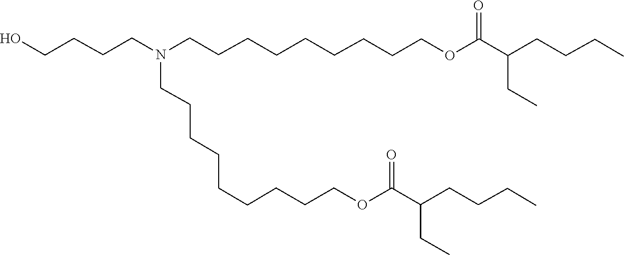 [SMILES: CCCCC(CC)C(=O)OCCCCCCCCCN(CCCCO)CCCCCCCCCOC(=O)C(CC)CCCC] | F |
| III-25 | 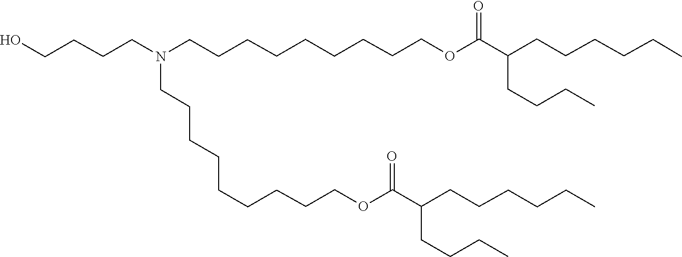 [SMILES: CCCCCCC(CCCC)C(=O)OCCCCCCCCCN(CCCCO)CCCCCCCCCOC(=O)C(CCCC)CCCCCC] | F |
| III-26 | 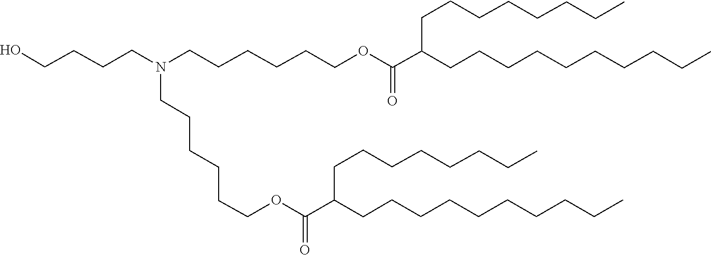 [SMILES: CCCCCCCCCCC(CCCCCCCC)C(=O)OCCCCCCN(CCCCO)CCCCCCOC(=O)C(CCCCCCCC)CCCCCCCCCC] | F |
| III-27 | 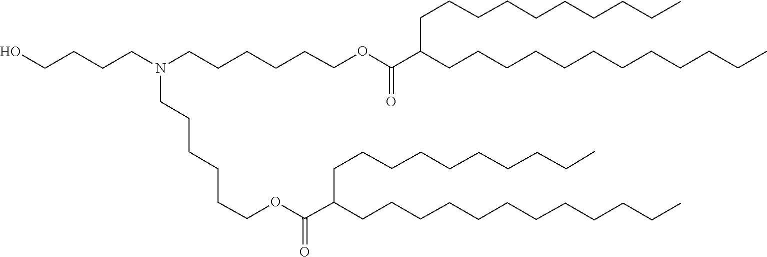 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] | F |
| III-28 | 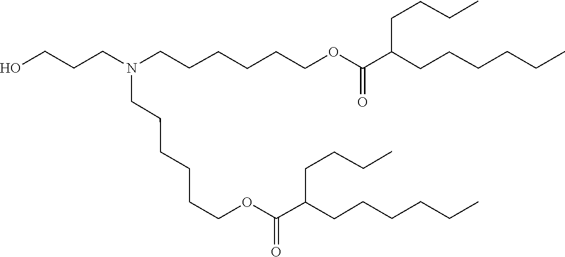 [SMILES: CCCCCCC(CCCC)C(=O)OCCCCCCN(CCCO)CCCCCCOC(=O)C(CCCC)CCCCCC] | F |
| III-29 | 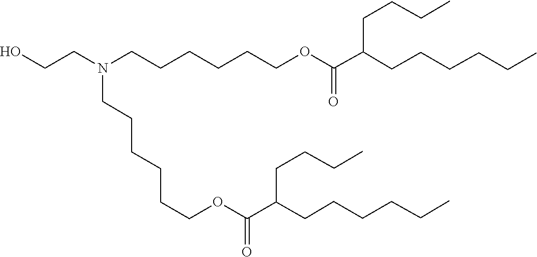 [SMILES: CCCCCCC(CCCC)C(=O)OCCCCCCN(CCO)CCCCCCOC(=O)C(CCCC)CCCCCC] | F |
| III-30 | 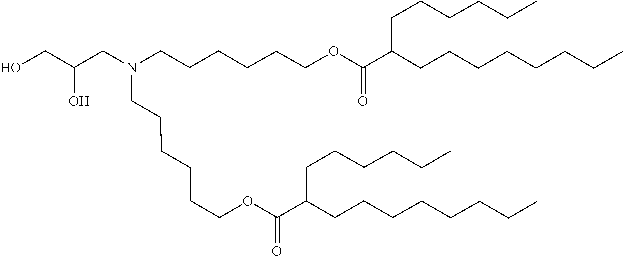 [SMILES: CCCCCCCCC(CCCCCC)C(=O)OCCCCCCN(CCCCCCOC(=O)C(CCCCCC)CCCCCCCC)CC(O)CO] | F |
| III-31 | 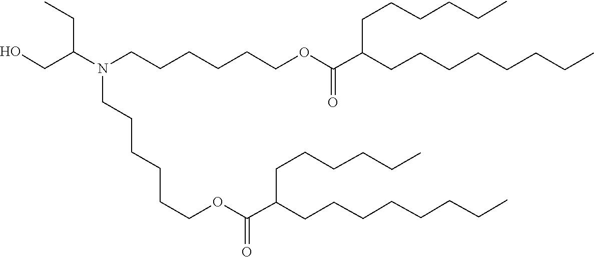 [SMILES: CCCCCCCCC(CCCCCC)C(=O)OCCCCCCN(CCCCCCOC(=O)C(CCCCCC)CCCCCCCC)C(CC)CO] | F |
| III-32 | 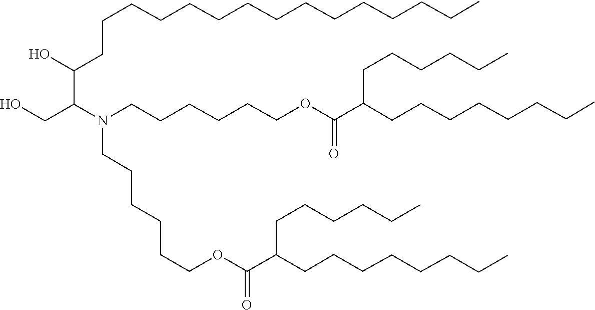 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] | F |
| III-33 | 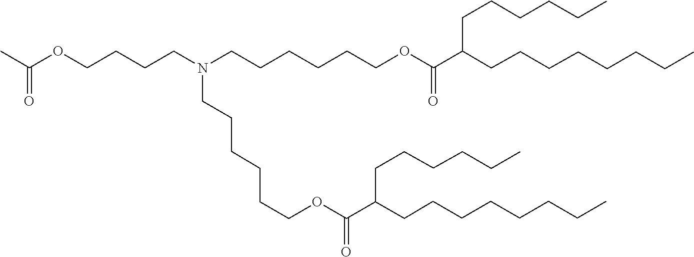 [SMILES: CCCCCCCCC(CCCCCC)C(=O)OCCCCCCN(CCCCCCOC(=O)C(CCCCCC)CCCCCCCC)CCCCOC(C)=O] | F |
| III-34 | 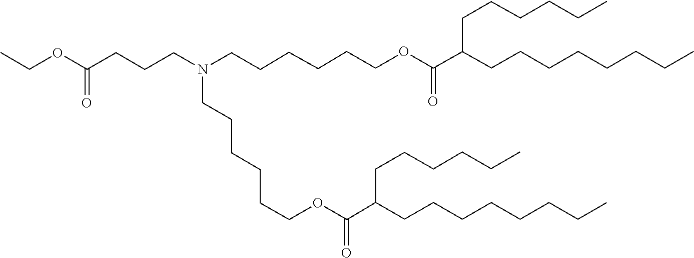 [SMILES: CCCCCCCCC(CCCCCC)C(=O)OCCCCCCN(CCCCCCOC(=O)C(CCCCCC)CCCCCCCC)CCCC(=O)OCC] | F |
| III-35 | 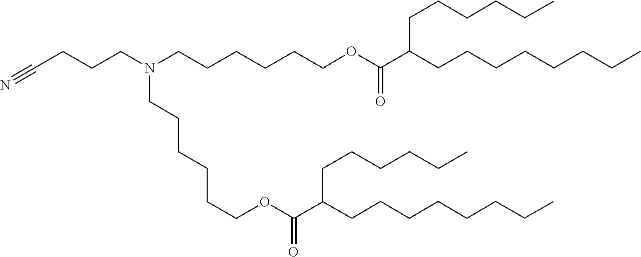 [SMILES: CCCCCCCCC(CCCCCC)C(=O)OCCCCCCN(CCCC#N)CCCCCCOC(=O)C(CCCCCC)CCCCCCCC] | F |
| III-36 | 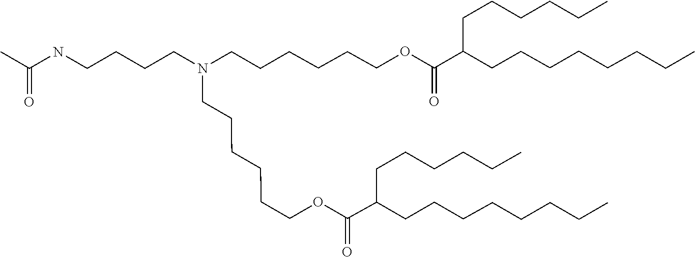 | F |

In some embodiments, the LNPs comprise a lipid of Formula (III), a nucleoside-modified RNA and one or more excipient selected from neutral lipids, steroids and pegylated lipids. In some embodiments the lipid of Formula (III) is compound III-3. In some embodiments the lipid of Formula (III) is compound III-7.

In some embodiments, the cationic lipid is present in the LNP in an amount from about 30 to about 95 mole percent. In one embodiment, the cationic lipid is present in the LNP in an amount from about 30 to about 70 mole percent. In one embodiment, the cationic lipid is present in the LNP in an amount from about 40 to about 60 mole percent. In one embodiment, the cationic lipid is present in the LNP in an amount of about 50 mole percent. In one embodiment, the LNP comprises only cationic lipids.

In some embodiments, the LNP comprises one or more additional lipids which stabilize the formation of particles during their formation.

Suitable stabilizing lipids include neutral lipids and anionic lipids.

The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1.

In various embodiments, the LNPs further comprise a steroid or steroid analogue. A “steroid” is a compound comprising the following carbon skeleton:

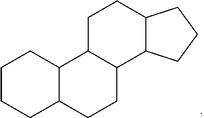 [SMILES: C1CCC2C(C1)CCC1C3CCCC3CCC21]

In some embodiments, the steroid or steroid analogue is cholesterol. In some of these embodiments, the molar ratio of the cationic lipid (e.g., lipid of Formula (I)) to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In some embodiments, the LNP comprises glycolipids (e.g., monosialoganglioside GM1). In some embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In some embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the cationic lipid to the pegylated lipid ranges from about 100:1 to about 25:1.

In some embodiments, the LNPs comprise a pegylated lipid having the following structure (IV):

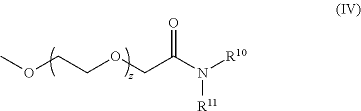

or a pharmaceutically acceptable salt, tautomer or stereoisomer thereof, wherein:
R¹⁰ and R¹¹ are each independently a straight or branched, saturated or unsaturated alkyl chain containing from 10 to 30 carbon atoms, wherein the alkyl chain is optionally interrupted by one or more ester bonds; and
z has mean value ranging from 30 to 60.

In some of the foregoing embodiments of the pegylated lipid (IV), R¹⁰ and R¹¹ are not both n-octadecyl when z is 42. In some other embodiments, R¹⁰ and R¹¹ are each independently a straight or branched, saturated or unsaturated alkyl chain containing from 10 to 18 carbon atoms. In some embodiments, R¹⁰ and R¹¹ are each independently a straight or branched, saturated or unsaturated alkyl chain containing from 12 to 16 carbon atoms. In some embodiments, R¹⁰ and R¹¹ are each independently a straight or branched, saturated or unsaturated alkyl chain containing 12 carbon atoms. In some embodiments, R¹⁰ and R¹¹ are each independently a straight or branched, saturated or unsaturated alkyl chain containing 14 carbon atoms. In other embodiments, R¹⁰ and R¹¹ are each independently a straight or branched, saturated or unsaturated alkyl chain containing 16 carbon atoms. In still more embodiments, R¹⁰ and R¹¹ are each independently a straight or branched, saturated or unsaturated alkyl chain containing 18 carbon atoms. In still other embodiments, R¹⁰ is a straight or branched, saturated or unsaturated alkyl chain containing 12 carbon atoms and R¹¹ is a straight or branched, saturated or unsaturated alkyl chain containing 14 carbon atoms.

In various embodiments, z spans a range that is selected such that the PEG portion of (II) has an average molecular weight of about 400 to about 6000 g/mol. In some embodiments, the average z is about 45.

In other embodiments, the pegylated lipid has one of the following structures:

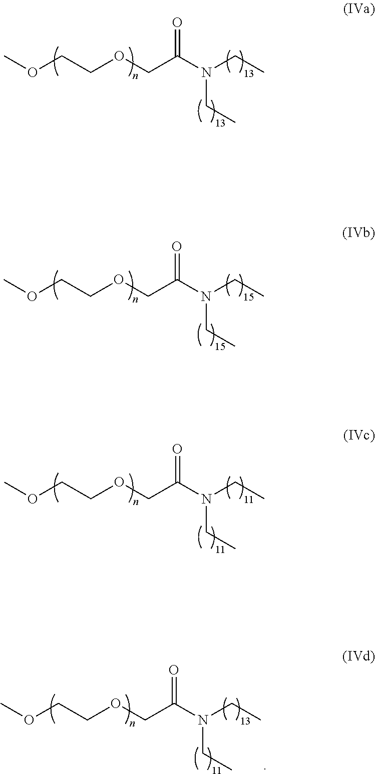

wherein n is an integer selected such that the average molecular weight of the pegylated lipid is about 2500 g/mol.

In some embodiments, the additional lipid is present in the LNP in an amount from about 1 to about 10 mole percent. In one embodiment, the additional lipid is present in the LNP in an amount from about 1 to about 5 mole percent. In one embodiment, the additional lipid is present in the LNP in about 1 mole percent or about 1.5 mole percent.

In some embodiments, the LNPs comprise a lipid of Formula (I), a nucleoside-modified RNA, a neutral lipid, a steroid and a pegylated lipid. In some embodiments the lipid of Formula (I) is compound 1-6. In different embodiments, the neutral lipid is DSPC. In other embodiments, the steroid is cholesterol. In still different embodiments, the pegylated lipid is compound IVa.

In some embodiments, the LNP comprises one or more targeting moieties, which are capable of targeting the LNP to a cell or cell population. For example, in one embodiment, the targeting moiety is a ligand, which directs the LNP to a receptor found on a cell surface.

In some embodiments, the LNP comprises one or more internalization domains. For example, in one embodiment, the LNP comprises one or more domains, which bind to a cell to induce the internalization of the LNP. For example, in one embodiment, the one or more internalization domains bind to a receptor found on a cell surface to induce receptor-mediated uptake of the LNP. In some embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo.

Other exemplary LNPs and their manufacture are described in the art, for example in U.S. Patent Application Publication No. US20120276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

The following Reaction Schemes illustrate methods to make lipids of Formula (I), (II) or (III).

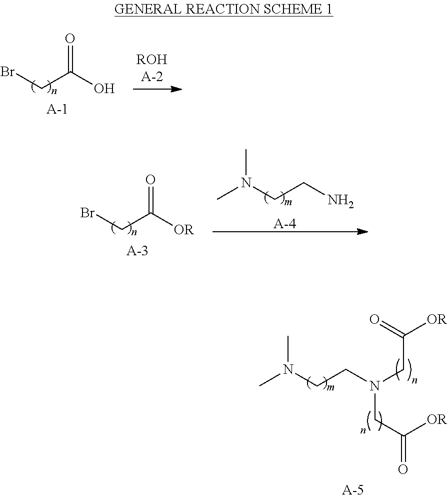

Embodiments of the lipid of Formula (I) (e.g., compound A-5) can be prepared according to General Reaction Scheme 1 (“Method A”), wherein R is a saturated or unsaturated C1-C24 alkyl or saturated or unsaturated cycloalkyl, m is 0 or 1 and n is an integer from 1 to 24. Referring to General Reaction Scheme 1, compounds of structure A-I can be purchased from commercial sources or prepared according to methods familiar to one of ordinary skill in the art. A mixture of A-1, A-2 and DMAP is treated with DCC to give the bromide A-3. A mixture of the bromide A-3, a base (e.g., N,N-diisopropylethylamine) and the N,N-dimethyldiamine A-4 is heated at a temperature and time sufficient to produce A-5 after any necessarily workup and or purification step.

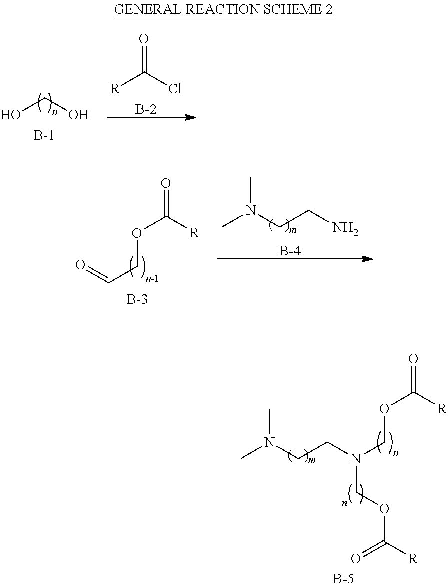

Other embodiments of the compound of Formula (I) (e.g., compound B-5) can be prepared according to General Reaction Scheme 2 (“Method B”), wherein R is a saturated or unsaturated C1-C24 alkyl or saturated or unsaturated cycloalkyl, m is 0 or 1 and n is an integer from 1 to 24. As shown in General Reaction Scheme 2, compounds of structure B—I can be purchased from commercial sources or prepared according to methods familiar to one of ordinary skill in the art. A solution of B-1 (1 equivalent) is treated with acid chloride B-2 (1 equivalent) and a base (e.g., triethylamine). The crude product is treated with an oxidizing agent (e.g., pyridinum chlorochromate) and intermediate product B-3 is recovered. A solution of crude B-3, an acid (e.g., acetic acid), and N,N-dimethylaminoamine B-4 is then treated with a reducing agent (e.g., sodium triacetoxyborohydride) to obtain B-5 after any necessary work up and/or purification.

It should be noted that although starting materials A-1 and B-1 are depicted above as including only saturated methylene carbons, starting materials which include carbon-carbon double bonds may also be employed for preparation of compounds which include carbon-carbon double bonds.

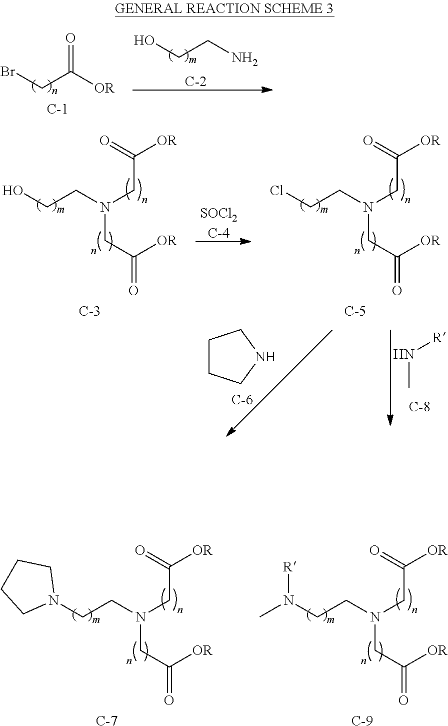

Different embodiments of the lipid of Formula (I) (e.g., compound C-7 or C9) can be prepared according to General Reaction Scheme 3 (“Method C”), wherein R is a saturated or unsaturated C1-C24 alkyl or saturated or unsaturated cycloalkyl, m is 0 or 1 and n is an integer from 1 to 24. Referring to General Reaction Scheme 3, compounds of structure C-1 can be purchased from commercial sources or prepared according to methods familiar to one of ordinary skill in the art.

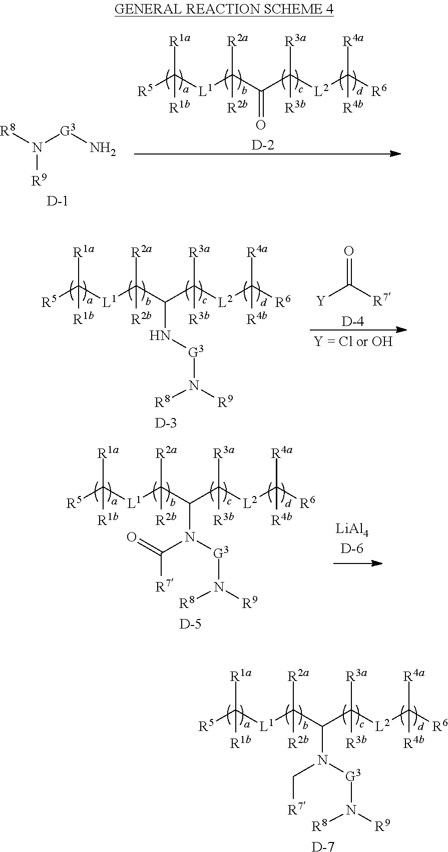

Embodiments of the compound of Formula (II) (e.g., compounds D-5 and D-7) can be prepared according to General Reaction Scheme 4 (“Method D”), wherein R^{1a}, R^{1b}, R^{2a}, R^{2b}, R^{3a}, R^{3b}, R^{4a}, R^{4b}, R⁵, R⁶, R⁸, R⁹, L¹, L², G¹, G², G³, a, b, c and d are as defined herein, and R^{7′} represents R⁷ or a C3-C19 alkyl. Referring to General Reaction Scheme 1, compounds of structure D-1 and D-2 can be purchased from commercial sources or prepared according to methods familiar to one of ordinary skill in the art. A solution of D-1 and D-2 is treated with a reducing agent (e.g., sodium triacetoxyborohydride) to obtain D-3 after any necessary work up. A solution of D-3 and a base (e.g. trimethylamine, DMAP) is treated with acyl chloride D-4 (or carboxylic acid and DCC) to obtain D-5 after any necessary work up and/or purification. D-5 can be reduced with LiAlH4 D-6 to give D-7 after any necessary work up and/or purification.

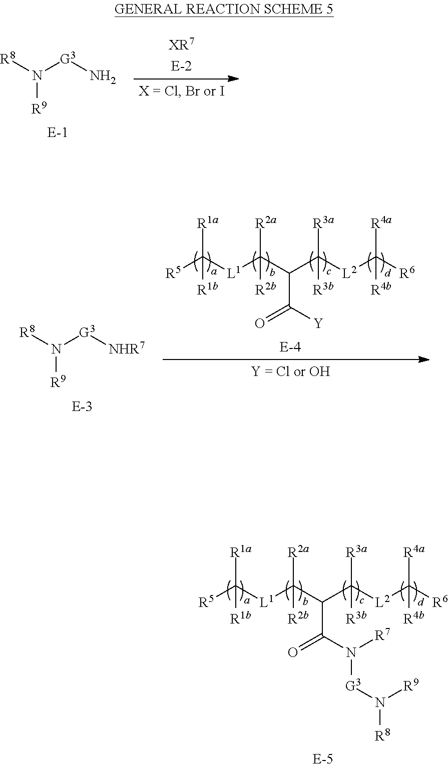

Embodiments of the lipid of Formula (II) (e.g., compound E-5) can be prepared according to General Reaction Scheme 5 (“Method E”), wherein R^{1a}, R^{1b}, R^{2a}R^{2b}, R^{3a}, R^{3b}, R^{4a}, R^{4b}, R⁵, R⁶, R⁷, R⁸, R⁹, L¹, L², G³, a, b, c and d are as defined herein. Referring to General Reaction Scheme 2, compounds of structure E-1 and E-2 can be purchased from commercial sources or prepared according to methods familiar to one of ordinary skill in the art. A mixture of E-1 (in excess), E-2 and a base (e.g., potassium carbonate) is heated to obtain E-3 after any necessary work up. A solution of E-3 and a base (e.g. trimethylamine, DMAP) is treated with acyl chloride E-4 (or carboxylic acid and DCC) to obtain E-5 after any necessary work up and/or purification.

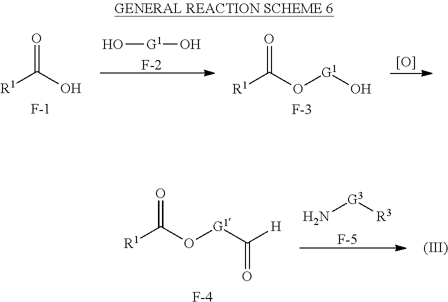

General Reaction Scheme 6 provides an exemplary method (Method F) for preparation of Lipids of Formula (III). G1, G3, R1 and R3 in General Reaction Scheme 6 are as defined herein for Formula (III), and G1′ refers to a one-carbon shorter homologue of G1. Compounds of structure F-1 are purchased or prepared according to methods known in the art. Reaction of F-1 with diol F-2 under appropriate condensation conditions (e.g., DCC) yields ester/alcohol F-3, which can then be oxidized (e.g., PCC) to aldehyde F-4. Reaction of F-4 with amine F-5 under reductive amination conditions yields a lipid of Formula (III).

It should be noted that various alternative strategies for preparation of lipids of Formula (III) are available to those of ordinary skill in the art. For example, other lipids of Formula (III) wherein L¹ and L² are other than ester can be prepared according to analogous methods using the appropriate starting material. Further, General Reaction Scheme 6 depicts preparation of a lipids of Formula (III), wherein G1 and G2 are the same; however, this is not a required aspect of the invention and modifications to the above reaction scheme are possible to yield compounds wherein G1 and G2 are different.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R¹¹ (where R¹¹ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers. In some embodiments, the formulation may comprise dry particles which comprise the active ingredient and which have a diameter in the range from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some instances having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Scaffold Compositions

In one aspect, the present invention provides a composition comprising a scaffold comprising one or more nucleic acid molecules encoding neuroprotective proteins, or fragments or variants thereof, as described herein. For example, in one embodiment, the scaffold comprises one or more nucleoside modified RNA encoding one or more neuroprotective proteins, or fragments or variants thereof, as described herein. In one embodiment, the scaffold comprises one or more LNPs encapsulating one or more nucleoside modified RNA encoding one or more neuroprotective proteins, or fragments or variants thereof, as described herein. Exemplary scaffold compositions include, but are not limited to, hydrogels, electrospun scaffolds, and combinations thereof. In certain embodiments, the scaffold is biocompatible. In certain embodiments, the scaffold is biodegradable. In certain embodiments, the scaffold comprises one or more cells embedded within the scaffold or cultured along the surface of the scaffold. For example, in one embodiment, the scaffold comprises periodontal ligament cells, or precursors thereof.

In certain embodiments, the scaffold comprises one or more extracellular matrix material and/or blends of naturally occurring extracellular matrix material, including but not limited to collagen, fibrin, fibrinogen, thrombin, elastin, laminin, fibronectin, hyaluronic acid, chondroitin 4-sulfate, chondroitin 6-sulfate, dermatan sulfate, heparin sulfate, heparin, and keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. The scaffolds can further comprise one or more carbohydrates such as chitin, chitosan, alginic acids, and alginates such as calcium alginate and sodium alginate. These materials may be isolated from plant products, humans or other organisms or cells or synthetically manufactured. In some embodiments, the scaffolds can further comprise decellularized or demineralized tissue. In some embodiments, the scaffolds can further comprise synthetic peptides Also contemplated are crude extracts of tissue, extracellular matrix material, or extracts of non-natural tissue, alone or in combination. Extracts of biological materials, including but are not limited to cells, tissues, organs, and tumors may also be included.

In one embodiment, the scaffold comprises a polymer. Suitable polymers include but are not limited to: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO) and polyorthoesters or any other similar synthetic polymers that may be developed that are biologically compatible. Polymers with cationic moieties can also be used, such as poly(allyl amine), poly(ethylene imine), poly(lysine), and poly(arginine). The polymers may have any molecular structure including, but not limited to, linear, branched, graft, block, star, comb, and dendrimer structures.

In various embodiments, the scaffolds can include one or more therapeutics. The therapeutics can be natural or synthetic drugs, including but not limited to: analgesics, anesthetics, antifungals, antibiotics, anti-inflammatories, nonsteroidal anti-inflammatory drugs (NSAIDs), antimicrobials, antiseptics, antivirals, a colored or fluorescent imaging agent, corticoids (such as steroids), enzymes, growth factors, hormones, minerals, nutritional supplements, vitamins, and the like. The therapeutic agent may also be other small organic molecules, naturally isolated entities or their analogs, organometallic agents, chelated metals or metal salts, peptide-based drugs, or peptidic or non-peptidic receptor targeting or binding agents. In some embodiments, the scaffolds can further comprise a delivery vehicle, such as nanoparticles, microparticles, liposomes, viral and non-viral transfection systems.

In one embodiment, the scaffold comprises a cell or cell population. The cell or cell population can include any cell that contributes to spinal cord injury repair. differentiated and undifferentiated stem cells. In some embodiments, the population of cells is at least partially derived from a subject's own tissue. In some embodiments, the population of cells is at least partially derived from another subject within the same species as the treated subject. In some embodiments, the population of cells is at least partially derived from a mammalian species that is different from the subject. For example, the cells may be derived from organs of mammals such as humans, monkeys, dogs, cats, mice, rats, cows, horses, pigs, goats and sheep.

In certain embodiments, the cell or cell population is genetically modified. In certain embodiments, the cells are genetically modified to express one or more of the neuroprotective proteins described herein. In one embodiment, the cells are modified to comprise a nucleic acid molecule encoding the one or more neuroprotective proteins described herein. For example, in one embodiment, the cells are modified to comprise one or more nucleoside modified RNA encoding one or more neuroprotective proteins, or fragments or variants thereof.

### Treatment Methods

The present invention provides methods of inducing spinal cord injury repair in a subject comprising administering an effective amount of a composition comprising one or more isolated nucleic acids encoding one or more neuroprotective proteins, or fragments or variants thereof. In one embodiment, the method treats or prevents spinal cord injury in a subject.

In one embodiment, the composition is administered to a subject having spinal cord injury. In one embodiment, the composition is administered to a subject at risk for spinal cord injury. In certain embodiments, the subject has a spinal cord injury, or is at risk for having a spinal cord injury, that may be caused by any number of conditions or diseases.

In one embodiment, the method comprises administering a composition comprising one or more nucleoside-modified nucleic acid molecules encoding one or more neuroprotective proteins, or fragments or variants thereof. In one embodiment, the method comprises administering a composition comprising a first nucleoside-modified nucleic acid molecule encoding a first neuroprotective proteins, or a fragment or a variant thereof, and a second nucleoside-modified nucleic acid molecule encoding a second neuroprotective proteins, or a fragment or a variant thereof.

In one embodiment, the method comprises administering one or more compositions, each composition comprising one or more nucleoside-modified nucleic acid molecules encoding one or more neuroprotective proteins, or fragments or variants thereof. In one embodiment, the method comprises administering a first composition comprising one or more nucleoside-modified nucleic acid molecules encoding one or more neuroprotective proteins, or fragments or variants thereof; and administering a second composition comprising one or more nucleoside-modified nucleic acid molecules encoding one or more neuroprotective proteins, or fragments or variants thereof.

In some embodiments, the method of the invention allows for sustained expression of the one or more neuroprotective proteins, described herein, for at least several days following administration. In some embodiments, the method of the invention allows for sustained expression of the one or more neuroprotective proteins, described herein, for at least 2 weeks following administration. In some embodiments, the method of the invention allows for sustained expression of the one or more neuroprotective proteins, described herein, for at least 1 month following administration. However, the method, in some embodiments, also provides for transient expression, as in some embodiments, the nucleic acid is not integrated into the subject genome.

In some embodiments, the method comprises administering nucleoside-modified RNA, which provides stable expression of the one or more neuroprotective proteins described herein. In some embodiments, administration of nucleoside-modified RNA results in little to no innate immune response, while inducing periodontal tissue and/or bone regeneration.

In some embodiments, the method provides sustained spinal cord injury repair. For example, in some embodiments, the method provides sustained spinal cord injury repair for more than 2 weeks. In some embodiments, the method provides sustained spinal cord injury repair for 1 month or more. In some embodiments, the method provides sustained spinal cord injury repair for 2 months or more. In some embodiments, the method provides sustained spinal cord injury repair for 3 months or more. In some embodiments, the method provides sustained spinal cord injury repair for 4 months or more. In some embodiments, the method provides sustained spinal cord injury repair for 5 months or more. In some embodiments, the method provides sustained spinal cord injury repair for 6 months or more. In some embodiments, the method provides sustained spinal cord injury repair for 1 year or more.

In one embodiment, a single administration of the composition induces sustained spinal cord injury repair for 1 month or more, 2 months or more, 3 months or more, 4 months or more, 5 months or more, 6 months or more, or 1 year or more.

Administration of the compositions of the invention in a method of treatment can be achieved in a number of different ways, using methods known in the art. In one embodiment, the method of the invention comprises systemic administration of the subject, including for example enteral or parenteral administration. In some embodiments, the method comprises oral delivery of the composition. In another embodiment, the method comprises intravenous delivery of the composition. In one embodiment, the method comprises injection of the composition into the spina column and/or spinal cord of a subject. In one embodiment, the method comprises administration of a scaffold composition to the spinal column and/or spinal cord of a subject.

It will be appreciated that the composition of the invention may be administered to a subject either alone, or in conjunction with another agent.

The therapeutic and prophylactic methods of the invention thus encompass the use of pharmaceutical compositions encoding one or more neuroprotective proteins, or fragments or variants thereof, described herein to practice the methods of the invention. The pharmaceutical compositions useful for practicing the invention may be administered to deliver a dose of from 1 ng/kg/day and 100 mg/kg/day. In one embodiment, the invention envisions administration of a dose, which results in a concentration of the compound of the present invention from 10 nM and 10 μM in a mammal.

Typically, dosages which may be administered in a method of the invention to a mammal, such as a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 0.1 μg to about 10 mg per kilogram of body weight of the mammal. In some embodiments, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months, several years, or even less frequently, such as every 10-20 years, 15-30 years, or even less frequently, such as every 50-100 years. The frequency of the dose will be readily apparent to the skilled artisan and will depend upon any number of factors, such as, but not limited to, the type and severity of the disease being treated, the type and age of the mammal, etc.

In one embodiment, the invention includes a method comprising administering one or more compositions encoding one or more neuroprotective proteins, or fragments or variants thereof, described herein. In some embodiments, the method has an additive effect, wherein the overall effect of the administering the combination is approximately equal to the sum of the effects of administering each neuroprotective protein. In other embodiments, the method has a synergistic effect, wherein the overall effect of administering the combination is greater than the sum of the effects of administering each neuroprotective protein or adjuvant.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Methods

mRNA-LNP production: Codon-optimized enhanced green fluorescent protein (eGFP) and human interleukin 10 (hIL-10) were synthesized and cloned into the mRNA production plasmid as described (Freyn et al., 2020). mRNA production and LNP encapsulation was performed as described (Freyn et al., 2020). Briefly, mRNAs were transcribed to contain 101 nucleotide-long poly(A) tails. m1Ψ-5′-triphosphate (TriLink) instead of UTP was used to generate modified nucleoside-containing mRNA. Capping of the in vitro transcribed mRNAs was performed co-transcriptionally using the trinucleotide cap1 analog, CleanCap (TriLink). mRNA was purified by cellulose (Sigma-Aldrich) purification, as described (Baiersdorfer et al., 2019). All mRNAs were analyzed by agarose gel electrophoresis and were stored frozen at −20° C. Cellulose-purified m1Ψ-containing RNAs were encapsulated in LNPs using a self-assembly process as previously described wherein an ethanolic lipid mixture of ionizable cationic lipid, phosphatidylcholine, cholesterol and polyethylene glycol-lipid was rapidly mixed with an aqueous solution containing mRNA at acidic pH (Maier et al., 2013). The mean hydrodynamic diameter of mRNA-LNPs was −80 nm with a polydispersity index of 0.02-0.06 and an encapsulation efficiency of ˜95%. mRNA-LNPs were stored at −80° C. Two or three batches from each mRNA-LNP formulations were used in these studies and variability in vaccine efficacy were not observed.

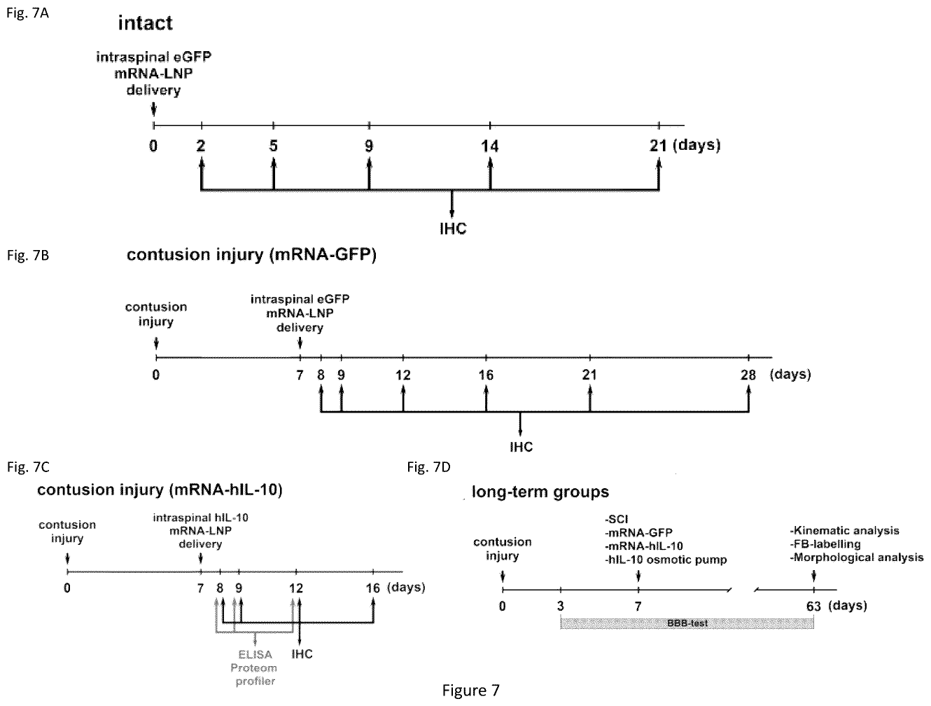

Spinal cord injury model and delayed intraspinal delivery of mRNA-LNP complexes: All animal surgery procedures and animal care were performed according to the Animal Care and Use Committee guideline. A rat contusion model of SCI was performed as described previously (Pajer et al., 2019, 2020). Sprague-Dawley female rats were anesthetized using (ketamine hydrochloride [Ketavet, 110 mg/kg body weight]; xylazine [Rompun, 12 mg/kg body weight]) and sterile precautions. A dorsal laminectomy was performed at the T10 vertebral level, the dura mater remained intact and the spinal cord was contused using an Infinity Horizon impactor (IH-0400, PSI LLC), applying 150 kdyn force. The superficial back muscle and the skin were sutured in layers. For postoperative animal care, saline (0.9%; 5 ml) to prevent dehydration and meloxicam (Metacam; 0.5 mg/kg body weight, Boehringer Ingelheim Vetmedica) were administrated. Their bladders were manually expressed three times a day until reflexive function was observed. At 7 days after the injury, 3.0 μg of mRNA-LNPs (eGFP-mRNA or hIL-10-mRNA) in Dulbecco's Phosphate Buffered Saline (PBS) were injected into animals intraspinally (5 l) with Hamilton pipette. Injured control animals (SCI group) received only saline (5 l). Experimental schematic of short and long term studies are shown in FIG. 7.

Administration of hIL-10 via osmotic pump: one week after the contusion injury, a miniosmotic pump (Alzet Osmotic Pumps, Cupertino, CA; type 1002, 100 μL volume, actively pumping for 2 weeks) filled with hIL-10 (4 μg/mL working concentration, all from R&D Systems, Minneapolis, MN) was placed subcutaneously in the dorsal region. A silicone tube (Degania Silicone Ltd, Kibbutz Degania, Israel, 0.3 mm in internal diameter) extended from the minipump to the spinal cord, and its distal end was inserted into the contusion cavity (Pajer et al., 2019). The tube was fixed to the surrounding musculature with 8-0 sutures (Ethilon) to avoid moving in or out of the spinal cord (Pajer et al., 2019).

Histo- and immunohistochemistry: After 20 minutes air-drying, the sections were permeabilized with 0.5% Triton X-100 in PBS for 5 minutes and blocked for 1 h at 24° C. with 5% BSA in PBS. Primary antibodies and lectin were used overnight at 4° C. as follows: chicken anti-GFP (1:1000, ab13970, Abcam), rabbit anti-IL-10 (1:400, ab34843, Abcam), rabbit anti-GFAP (1:500, 7260, Abcam), goat anti-GFAP (1:500, ab53554, Abcam), rabbit anti-TUBB3 (1:500, ab18207, Abcam), mouse anti-TUBB3 (1:500, ab7751, Abcam) and biotinylated Griffonia Simplicifolia isolectin B4 (GSA-IB4, 1:200, B1205, Vector Laboratories). The following secondary antibodies were used: biotinylated goat anti-rabbit IgG (1:200, BA-1000, Vector Laboratories). The immune reaction was completed by Alexa Fluor 488 goat anti-chicken (1:600, A11039, Thermo Fisher Scientific), Alexa Fluor 488 goat anti-rabbit (1:600, A11008, Thermo Fisher Scientific), Alexa Fluor 488 donkey anti-goat (1:600, A11055, Thermo Fisher Scientific), Alexa Fluor 546 donkey anti-rabbit (1:600, A10040, Thermo Fisher Scientific), Alexa Fluor 594 goat anti-mouse (1:600, A21203, Thermo Fisher Scientific) and Streptavidin Alexa Fluor 488 (1:600, S11223, Thermo Fisher Scientific). The sections were covered using Vectashield mounting medium containing DAPI (1.5 μg/ml; H-1000-10, Vector Laboratories), which labelled the nuclei of the cells. Negative controls for the secondary antibodies were performed by omitting the primary antibodies.

Immunoreactive sections were analyzed using a BX-41 epifluorescent microscope (Olympus) equipped with a DP-74 digital camera and its CellSens software (V1.18, Olympus), a Panoramic MIDI II slide scanner (3D Histech) with Panoramic Scanner 2.1.2 software (3D Histech) and an Olympus Fv-10i-W compact confocal microscope system (Olympus) with Fluoview Fv10i software (V2.1, Olympus).

Image analysis—quantification of GSA-B4 expression: for quantifying GSA-B4 reactivities, analysis was performed according to Pajer and coworkers (Pajer et al., 2019 and 2020). Two parasagittal sections (150 μm apart from each other) containing the lesion area were analysed, 8, 9 and 12 days after injury in SCI, mRNA-GFP and mRNA-hIL-10 group. Microphotographs were taken using an Olympus BX-41 epifluorescence microscope equipped with a DP-74 digital camera and the whole spinal cord section area including the lesion area and a 2 mm long extension of the tissue rostrally and caudally from the cavity ends was analysed using ImageJ software. The background intensity of unstained samples was individually subtracted from the intensity of treated sections. To correct for interanimal variations in the immunolabeling efficiency, the intensity of the immunolabeled tissue was normalized to the same section (uninjured area) 4 mm rostral to tip of the lesion area. Data were expressed as fold increase immunointensity normalized to uninjured value.

Proteom profiler arrays: the rat spinal cord fractions were homogenized in PBS with protease inhibitor (Sigma). After homogenization Triton X-100 (Sigma) was added to a final concentration of 1%. The samples were frozen for −75° C., thaw, and centrifuged at 10,000×g for 5 minutes. The supernatant was collected and the total protein concentration was determined using Pierce BCA Protein Assay Kit (Thermo Scientific). The blood samples were allowed to colt for 1 hour at room temperature and at 4° C. overnight. The sera were collected after centrifugation the blood at 1000×g for 5 minutes. The sample (spinal cord and serum) cytokine and chemokine production was determined using Proteome Profiler Rat Cytokine Array Kit, Panel A (R&D Systems). For the parallel determination of the relative levels of selected rat cytokines and chemokines, 390 μg total protein of spinal cord homogenization and 600 μl serum were used on each membrane. The spinal cord (130-130 μg total protein) samples and sera (200-200 μl) were pooled form 3-3 animals/group. The assay was performed following the manufacturer's instructions. The chemiluminescent signals from the bound cytokines present in the spinal cord and sera were detected using the LI-COR Odyssey Imaging System and analysed with Image Studio Software.

Human interleukin-10 enzyme-linked immunosorbent assay: The rat samples' human IL-10 content was evaluated using a human IL-10 ELISA Kit (Sigma). The spinal cord samples were diluted to 2 mg/ml total protein concentration; the sera were diluted two fold; and the samples were tested in duplicate. The experiment was performed following the factory sandwich ELISA instructions. Briefly, the diluted rat spinal cord and sera samples were run in 100 μl. After 2.5 hours incubation at room temperature the plate was washed and covered with Detection Antibody for one hour. The washing step was repeated, after which the Streptavidin Solution was added for 45 minutes. After washing the wells with 300 μl Wash Buffer 4 times, the TMB Substrate Solution was added, then, after 30 minutes incubation the reaction was stopped by adding the Stop Solution. The absorbance was measured at 450 nm.

Retrograde labelling and quantitative assessment of retrogradely labelled neurons: Retrograde labelling was performed as described previously (Pajer et al., 2019, 2020). Briefly, the L2-4 spinal segments were explored 9 weeks after the injury. At the level of L3 spinal segment a right hemisection was performed. Fast Blue (FB) crystals (Chemimart GmbH, Berlin, Germany) were placed into the gap and the wound was closed. Seven days after the application, the animals were reanesthetized and perfused transcardially. Transversal sections (30 μm thick) taken from the motor cortex, brainstem and spinal cord (C2, C6, T1 and T5 spinal segments) were cut in a cryostat (Leica CM-1850, Leica GmbH, Germany) and mounted onto gelatinized slides. Every transversal section from the T5, T1, C6 and C2 spinal segments and every 5th or every from the brainstem 10th coronal section from the brain were used, respectively.

Morphometric analysis of lesion area and spared tissue: Every four transverse section from the T8-L1 segments containing the lesion cavity was stained with cresyl-violet (1% aqueous cresyl-violet solution, C-1791, Sigma-Aldrich) (n=4 in each group). The border between the intact tissue and the lesion cavity composed of small cysts was defined. The whole cystic cross-sectional area (lesion cavity area) at the level of the epicentre was determined as follows: the number of pixels of the reference area (1 mm²) and that of the cystic area was computed through the use of the NIH ImageJ analysis software (imagej.nih.gov/ij). The pixel number of the cystic area was divided by that of the reference area and the result was expressed in percentage of lesion area compared to intact value.

The number of pixels of the spared tissue was measured at the epicentre (0) and 0.4, 0.8, 1.2, and 1.6 mm rostrally and caudally from it. Identical spinal cord segments of intact animals were used as reference values. The amount of spared tissue in the long term groups was given as percentage of intact spinal cord values.

Open field test for locomotor recovery: In the long term survival groups (9 weeks), motor function recovery was evaluated by BBB test (Basso et al., Journal of Neurotrauma, 1995 February; 12(1):1-21) at three days after the injury and once a week for 9 weeks. Two observers, unaware of experimental procedures, tested the animals in an open field (150×100 cm) for 4 min at a similar time of day for each testing.

Video-based motor functional analysis: In the 9th postoperative week a multi-parametric kinematic analysis was carried out with a custom-made system (Bellik et al., Scientific Reports 2020 Dec. 29; 10(1):22414, Torok et al., Journal Neuroscience Methods, 2021 Oct. 30: 109398). The method allows the measurement of different joint angles in different moments of the step cycle. To achieve this, two high speed cameras (one from lateral and one from rear aspect) and a mirror system were implemented surrounding a runway where the animals could walk into only one direction. The knee flexion, the ankle flexion, the knee lifting, the ankle lifting, and toe-off angle (TOA) parameters were recorded from lateral aspect together with the metatarsus-surface angle (MSA) and tibia-surface angle (TSA) observed from rear-view (Bellik et al., Scientific Reports 2020 Dec. 29; 10(1):22414, Torok et al., Journal Neuroscience Methods, 2021 Oct. 30: 109398).

Statistical analysis: student's t-test was used to compare two groups and one-way ANOVA with LSD post-hoc test to compare more than two groups. BBB scores were analysed using repeated measures ANOVA with LSD post-hoc test. The level of significance was set at p<0.05, and all error bars represent as standard error of mean (SEM).

### Results

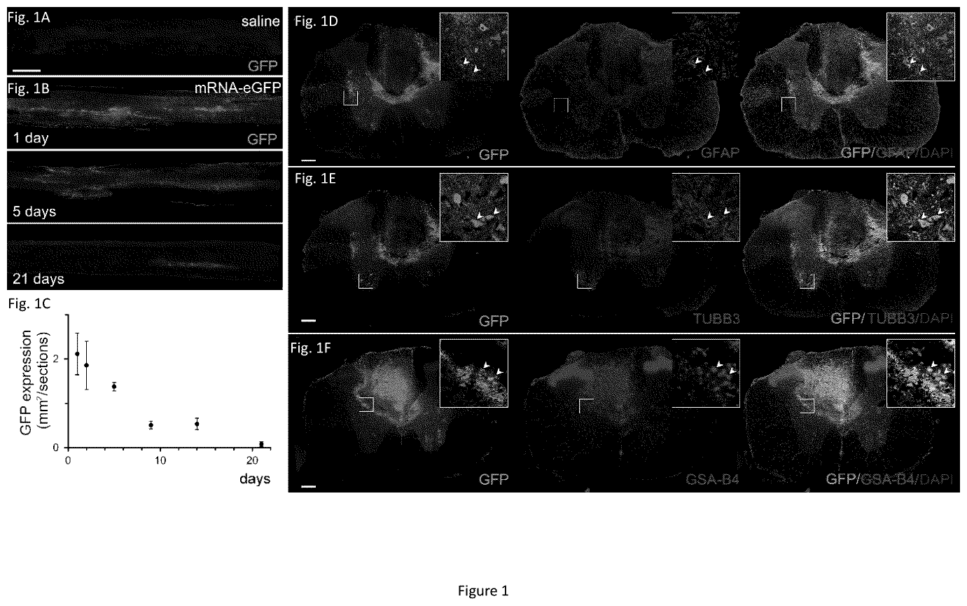

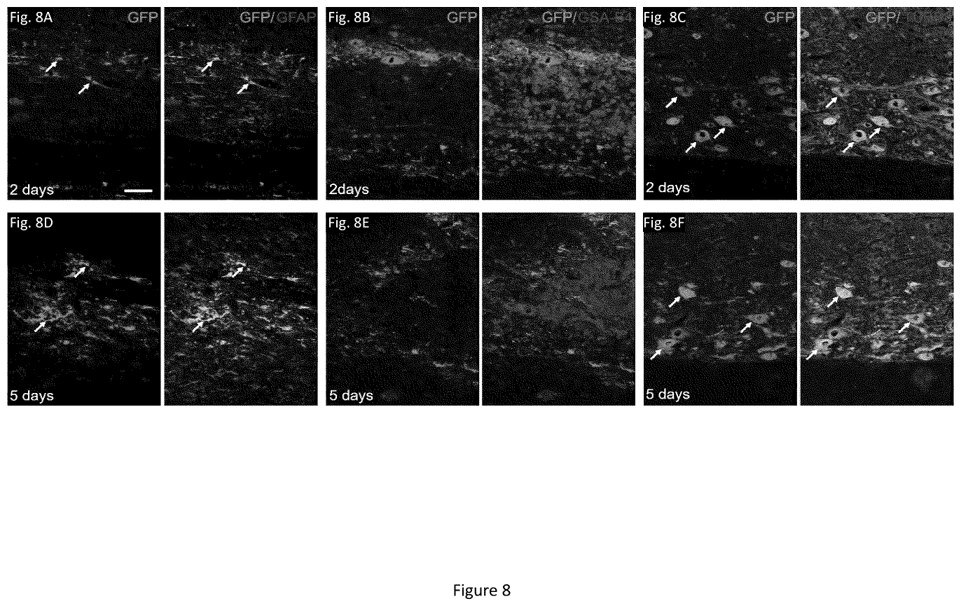

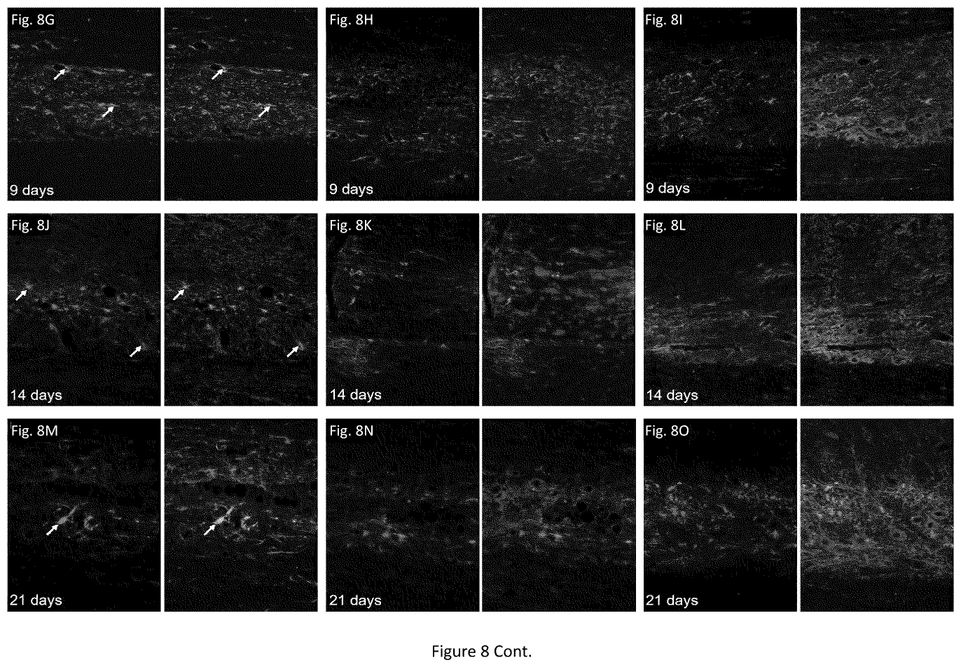

Intraspinal delivery of eGFP mRNA-LNPs resulted in transient protein production in intact spinal cord: Pardi et al. showed that a single injection of low doses of LNP-formulated firefly luciferase-encoding mRNA (0.1-5 μg) translated at high levels for up to 10 days depending on the dose and the site of the delivery in mice (Pardi et al., 2015). To examine the duration and distribution of protein production from mRNA-LNPs in the central nervous system, 3 μg eGFP mRNA-LNPs were administered into the intact rat spinal cord at the T10 vertebral level. Strong immunofluorescent signal was measured rostrally and caudally from the injection site for 5 days after injection (FIGS. 1A and 1B). FIG. 1A shows no eGFP expression in intact rat spinal cord without intraspinal administration of mRNA-LNP encoding eGFP. FIG. 1B shows eGFP expression in parasagittal section of rat spinal cord 1, 5 and 21 days after the intraspinal application of mRNA-LNPs. The highest amount of eGFP protein was detected 1 day post injection and eGFP signal could be measured for up to 21 days as shown in FIG. 1C. FIG. 1C shows quantification of the fluorescence signal of eGFP in rat spinal cord injected with 3.0 μg mRNA-LNPs. The eGFP protein was mainly detected in astrocytes (GFAP-positive cells) and neurons (TUBB3-positive cells) in the affected segment (FIGS. 1D and 1F). Nearby the stub channel, the microglia/macrophages (GSA-B4-positive cells) expressed eGFP 1 day after injection (FIG. 1E). Interestingly, eGFP was only produced by astrocytes and neurons 2 and 5 days after the mRNA-LNP administration (FIGS. 8A-8F). At later time points eGFP expression of neurons ceased but remained in astrocytes up to 21 days (FIGS. 8G-8O).

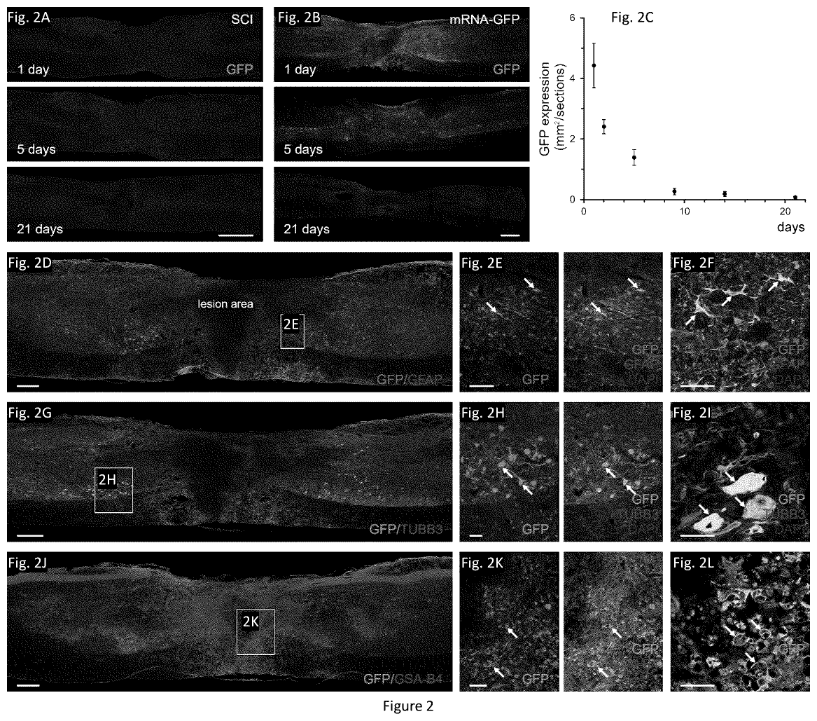

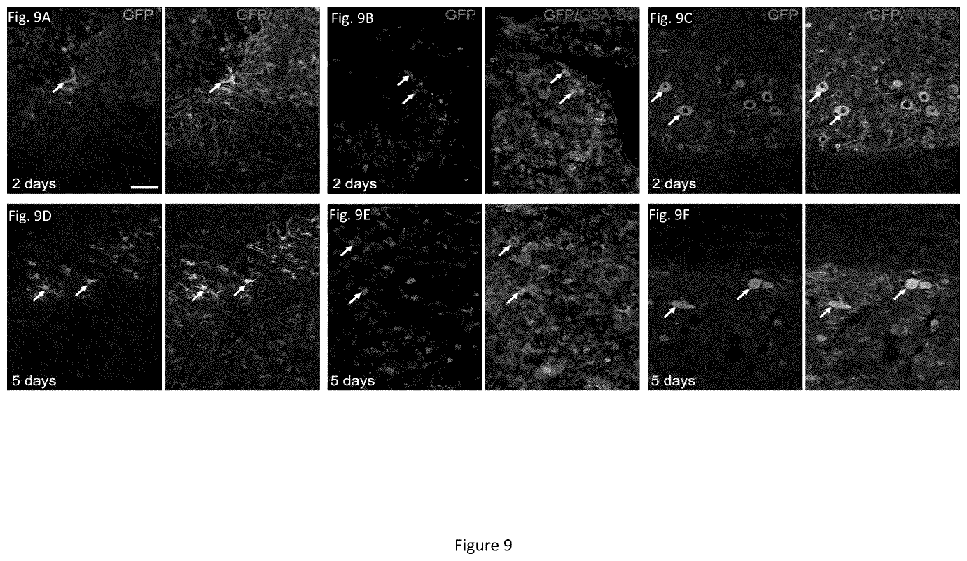

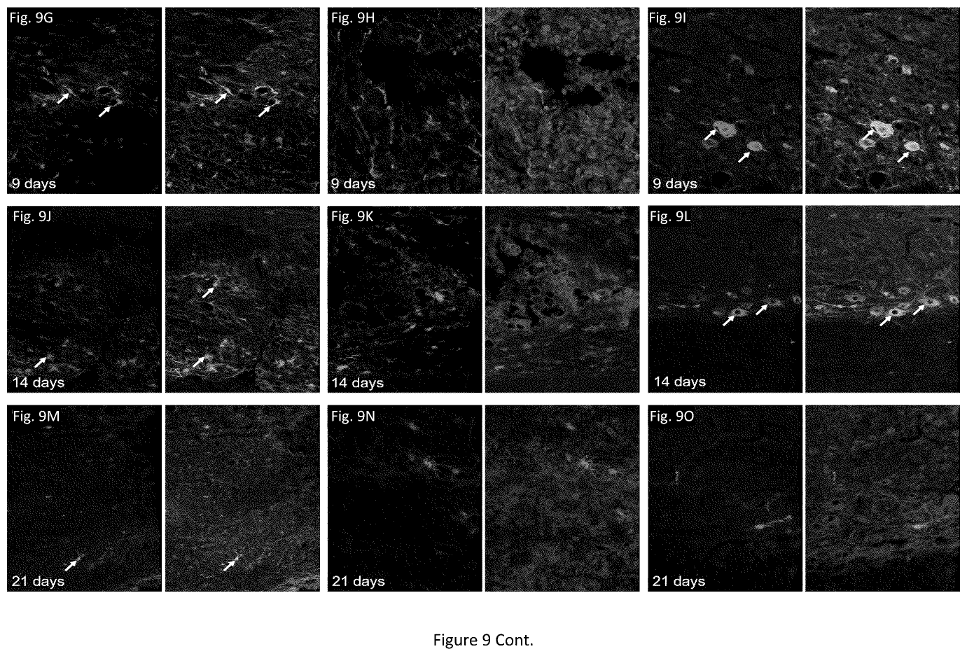

Protein expression after delayed intraspinal delivery of eGFP-mRNA-LNPs in injured spinal cord: The expression kinetics of eGFP in an injured spinal cord was examined. In injured spinal cord the eGFP expression was not detectable (FIG. 2A). Strong eGFP immunofluorescence signal was detected at the lesion site and rostrally and caudally from the end of the lesion following intraspinal delivery of mRNA-LNP encoding eGFP (FIG. 2B). The highest eGFP expression was measured at 1 days after the injection of mRNA-LNPs followed by a slow decrease in protein production up to 21 days (FIG. 2B). To identify the cells transfected by mRNA-LNPs, parasagittal spinal cord tissue sections were stained for GFAP, TUBB3 and GSA-B4 to identify astrocytes (GFAP), neurons (TUBB3) and microglia/macrophages (GSA-B4), as well as for eGFP. GFAP stains showed overlap with eGFP mainly around the lesion site (FIGS. 2D-2F). TUBB3-positive neurons and their processes expressed strongly eGFP rostrally and caudally from the lesion (FIGS. 2G-2I), while eGFP/GSA-B4-positive cells were mainly detected in the epicentre of the lesion (FIGS. 2J-2L). At 2 and 5 days after mRNA-LNP delivery, astrocytes, neurons and GSA-B4-positive cells expressed eGFP (FIGS. 9A and 9B). However, on day 9 and 14 after mRNA-LNP injection only neurons and astrocytes showed eGFP expression (FIGS. 9C and 9E), while on day 21 exclusively few astrocytes showed co-localization with eGFP protein (FIG. 9F). These results clearly showed that intraspinal administration of mRNA-LNPs into the injured spinal cord resulted in active and transient translation of mRNA to protein.

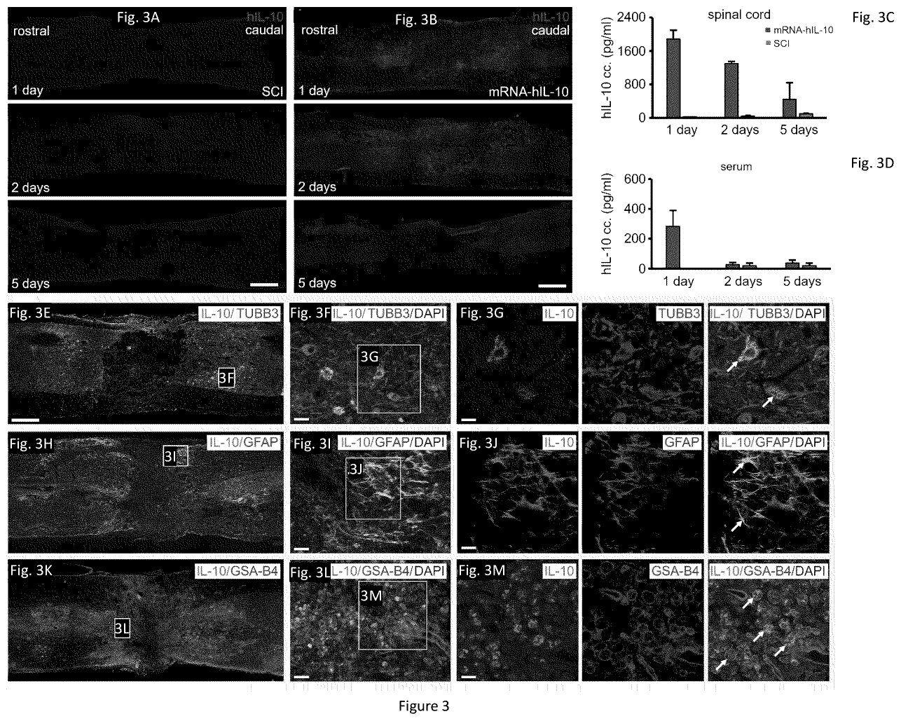

Expression kinetics of LNP-formulated hIL-10 mRNA translation in injured spinal cord: Based on the results above, anti-inflammatory hIL-10 expression was tested using delayed intraspinal delivery of hIL-10 mRNA-LNPs following spinal cord contusion injury in rats. Parasagittal sections of spinal cord were stained with hIL-10 specific antibody and strong immunofluorescence was detected rostrally and caudally from the injection site within the first 2 days and weak expression 5 days after the injection of mRNA-LNPs encoding hIL-10 (FIGS. 3A and 3B). The highest expression was detected on day 1 after the injection and there was a steady decline until day 5 in the spinal cord (FIG. 3C). Interestingly, hIL-10 was detected in the serum 1 day after intraspinal administration of mRNA but at later time points hIL-10 was barely evincible (FIG. 3D).

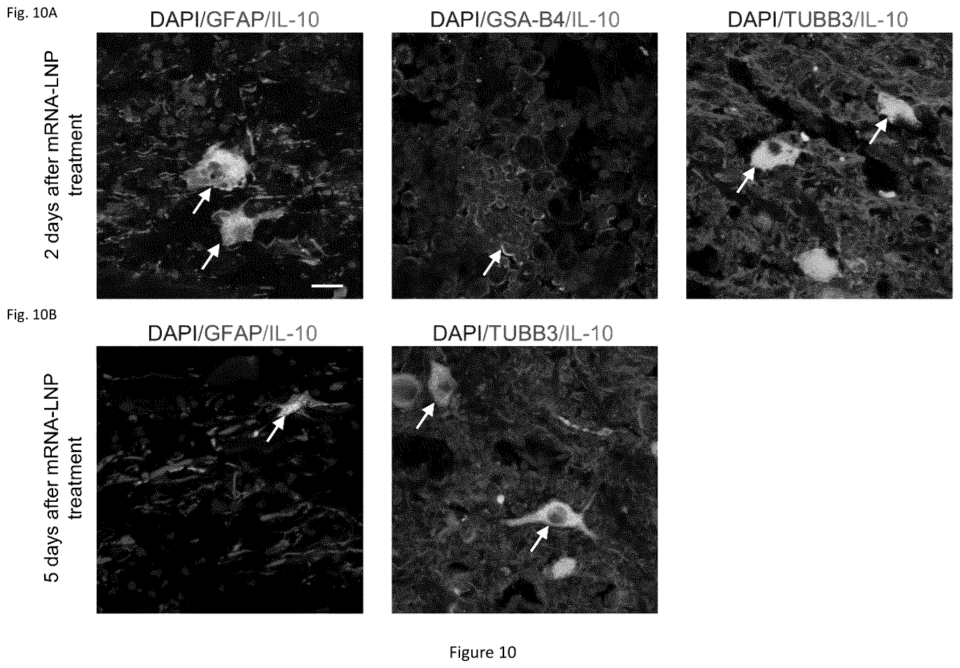

At day 1 after the mRNA-LNP injection, granular expression of hIL-10 was observed in the cell body of neurons in the affected segment (FIGS. 3E-3G). TUBB3-positive processes showed no overlap with hIL-10. Similar granular expression pattern of hIL-10 was observed in the astrocytes around the lesion area, respectively (FIGS. 3H-3J), while number of GSA-B4-positive microglia/macrophages overlapped with hIL-10 within the lesion (FIGS. 3K-3M). At 2 days after mRNA-LNP delivery, astroctyes, neurons and GSA-B4-positive cells strongly expressed hIL-10 (FIG. 10A). Five days after mRNA-LNP administration, hIL-10 protein expression was observed only in cell body of neurons and astrocytes close vicinity to the lesion (FIG. 10B). In contrast to the translation of mRNA encoding eGFP in the injured spinal cord, hIL-10 expression was not detected 9 or 14 days after the intraspinal administration. These results suggest that the resident cells of spinal cord and invading macrophages were transfected by the mRNA-LNPs encoding hIL-10 and least 5 days active protein expression was detected.

Expression of hGDNF, hIL-6, hIL-10 and hMIP-1 Alpha mRNA-LNP in the Injured Spinal Cord

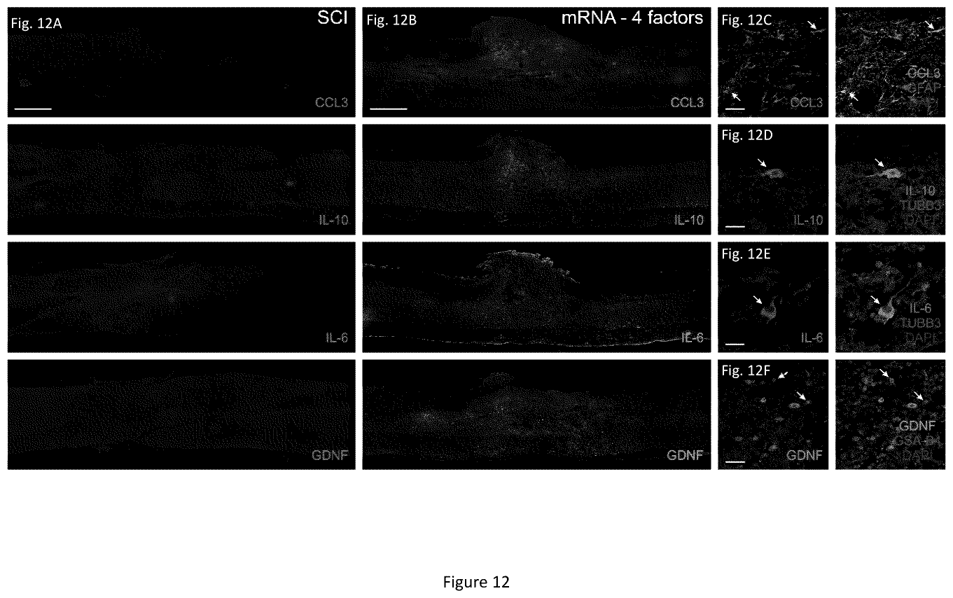

The expression of hGDNF, hIL-6, hIL-10 and hMIP-1 alpha was tested after delayed intralesional delivery of hGDNF, hIL-6, hIL-10 and hMIP-1 alpha mRNA-LNP following spinal cord contusion injury in rats. Paramedian sagittal sections of the spinal cords were immunostained with hGDNF, hIL-6, hIL-10 and hMIP-1 alpha-specific antibodies. None of the 4 factors were detected in injured control animals (SCI group, FIG. 12A) Immunofluorescence signal was detected in the lesion area and rostrally and caudally from the injection site on days 1 after the injection of mRNA-LNP (FIG. 12B). Strong protein expression was detected in astrocytes and neurons (FIGS. 12C-12E). GSA-B4-positive cells produced weakly the factors 1 day after mRNA-LNP administration (FIG. 12F).

These results suggest that the host cells of the spinal cord were transfected by the hGDNF, hIL-6, hIL-10 and hMIP-1 alpha mRNA-LNP leading to active protein synthesis.

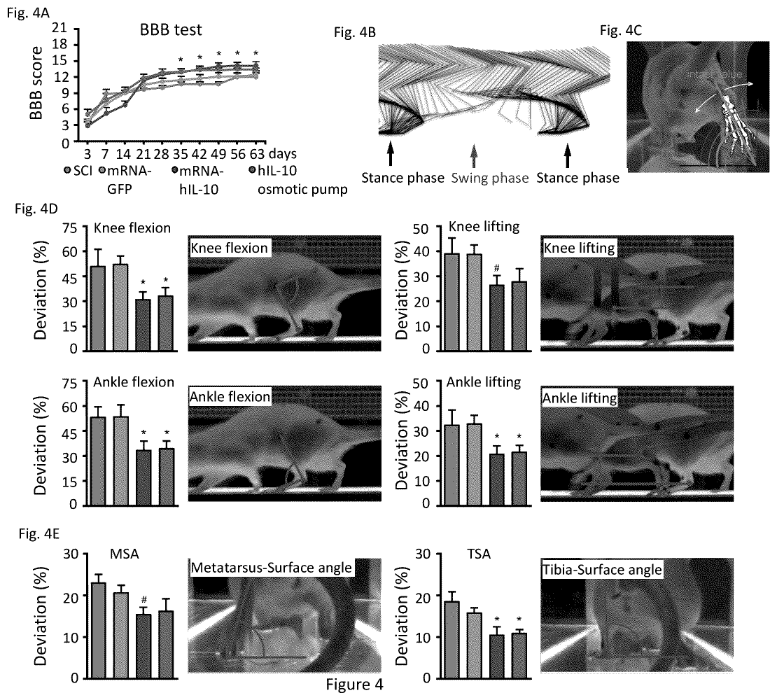

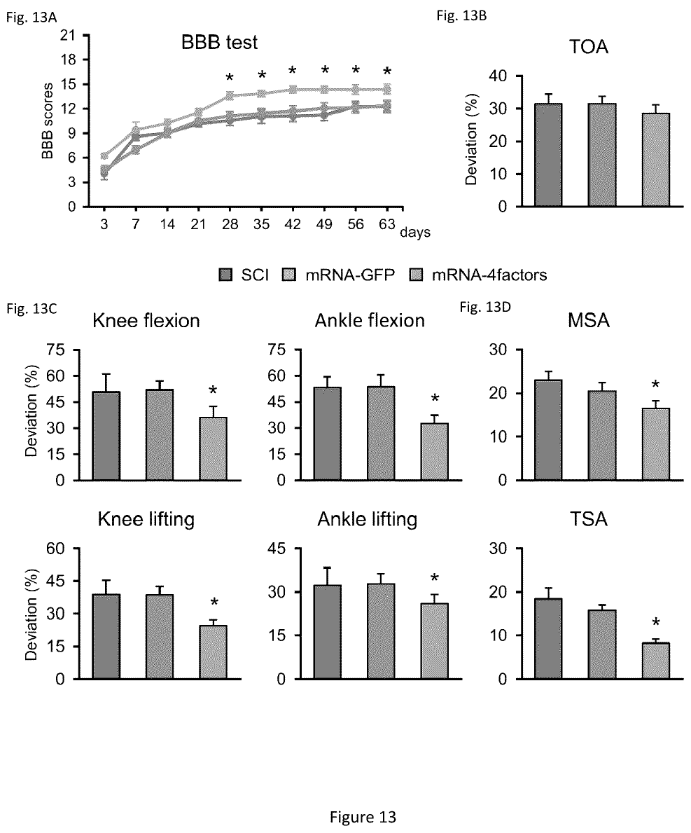

Improved locomotor pattern after treatment with mRNA-LNPs encoding hGDNF, hIL-6, hIL-10, and hMIP-1 alpha protein: Motor function recovery was estimated by visual observation according to BBB locomotor rating scale (Basso et al., 1995). At week 3 after the injury, a craggy increase of BBB score was observed in the hIL-10 mRNA-treated animals and further slightly motor improvement was detected in mRNA-hIL-10 group up to 9 weeks (FIG. 4A). In the other hIL-10 treated group (hIL-10 osmotic pump), animals showed similar locomotor pattern as mRNA hIL-10-treated group. A statistically significant difference was found between hIL-10 treated animals compared to the controls (SCI and mRNA-GFP groups; FIG. 4A). From week 4 after injury, a significant improvement based on the BBB score was observed in the mRNA-4 factors animals followed by a continuous motor improvement up to 9 weeks (FIG. 13A).

The kinematics analysis of treated (mRNA-hIL-10, hIL-10 osmotic pump groups, and 4-factor treated) and control rats (SCI and mRNA-GFP groups) was performed to provide a quantitative information about knee flexion and lifting, ankle flexion and lifting, metatarsus surface angle, tibia surface angle, and TSA at 9 weeks after the injury (FIGS. 4B-4D and 13B-13D). Consistent with the BBB results (9 weeks after injury), the kinematic analysis assessments revealed that hIL-10 treated animals (mRNA-hIL-10 and hIL-10 osmotic pump group) and 4-factor treated animals displayed a significant improvement in all examined parameters compared to control animals (SCI and mRNA-GFP groups) that displayed slight recovery after SCI.

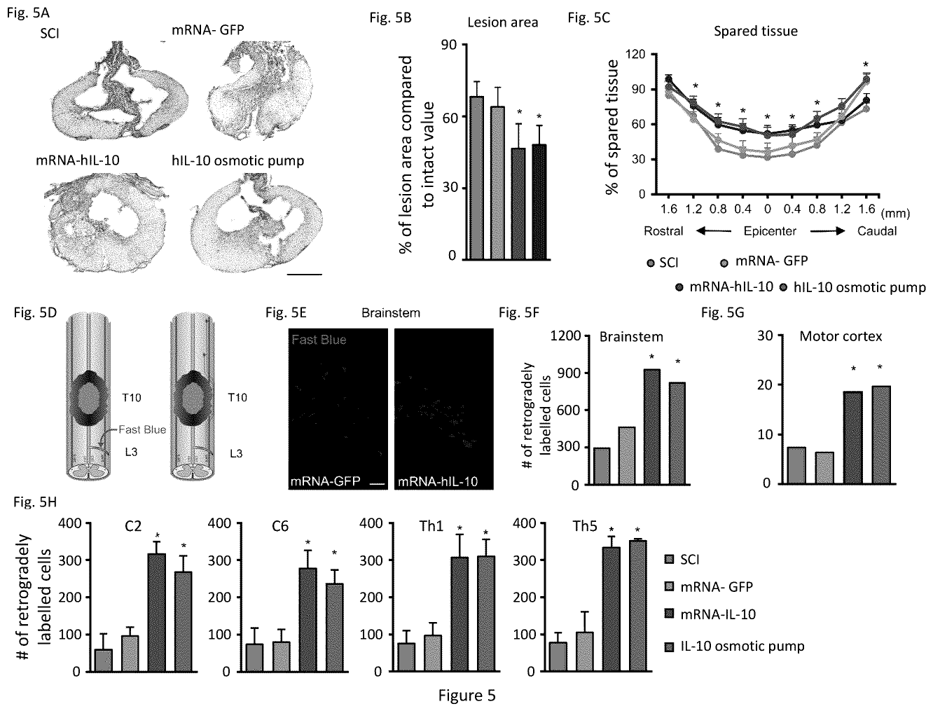

Morphological restoration following intraspinal delivery of mRNA-LNP encoding hIL-10: Morphometric analysis of lesion area at the epicentre and spared tissue were preformed 9 weeks after the injury. Within the lesion area a rostro-caudally extended cavity was observed with cellular debris in SCI groups (FIG. 5A). Administration of hIL-10 (mRNA-hIL-10 and hIL-10 osmotic pump groups) resulted in significantly lesser lesion area at the epicentre of the injury and enhanced significantly greater amount of tissue sparing (FIGS. 5B and 5C).

Next, it was evaluated whether hIL-10 mRNA treatment improves the connections between the segments caudal to the lesion and various cranial parts of the CNS. To study proprio- and supraspinal connections of injured spinal cord, retrograde tracer (Fast Blue) was placed caudally to the injury into the right L³ segment and the numbers of retrogradely labelled neuronal somata in the spinal cord, brainstem and somatomotor cortex were evaluated (Figured 5D and 5E). In mRNA-hIL-10 group significantly higher number of retrogradely labelled cells could be found in the brainstem and in the motor cortex compared to controls (SCI and mRNA-GFP groups; FIGS. 5F and 5G). Significantly higher numbers of FB-labelled propriospinal neurons were found in the Th5, Th1, C6, and C2 spinal segments in animals treated with hIL-10 (mRNA-hIL-10 and hIL-10 osmotic pump group) than in their controls (SCI and mRNA-GFP groups; FIG. 5H). The number of retrogradely labelled neurons in C2 and C6 spinal segment of the mRNA-hIL-10 treated animals was higher than in the hIL-10 osmotic pump group but this difference did not achieve statistical significance.

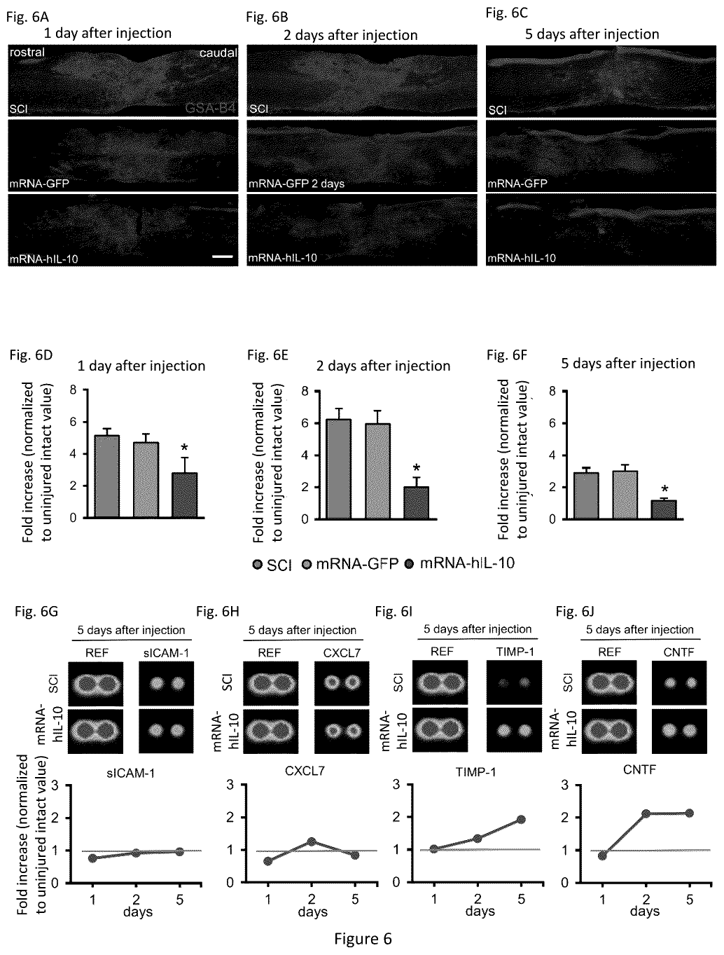

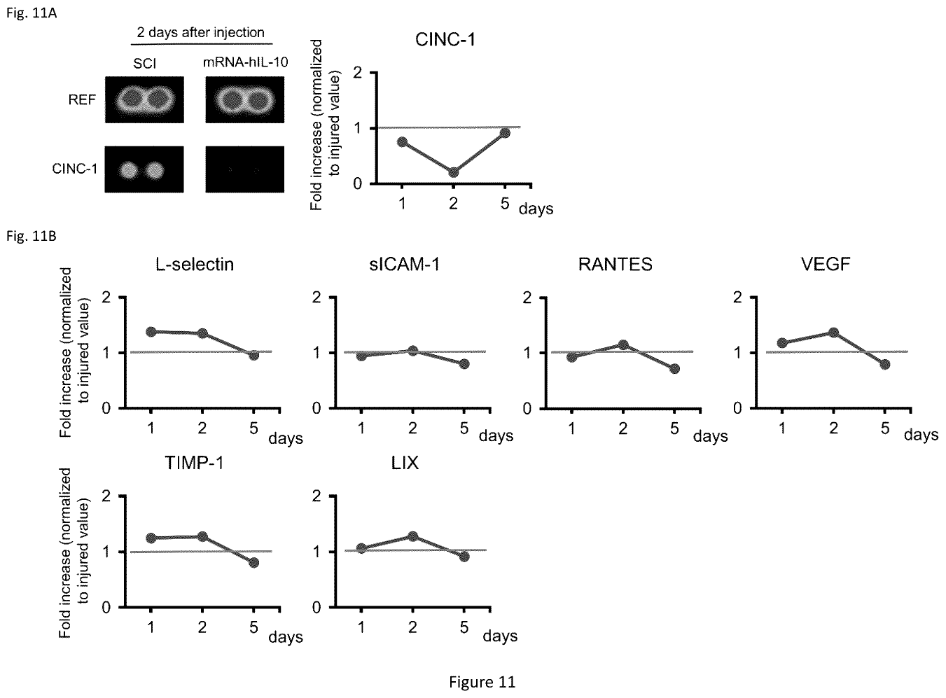

mRNA-LNP encoding hIL-10 treatment decreases the microglia/macrophage reaction and induces cytokine changes in injured spinal cord: LNPs used in many preclinical studies are highly inflammatory. Therefore, it was investigated whether the intraspinal administration of LNP alters the microenvironment of the lesion area. The densities of microglia/macrophages (GSA-B4) were examined and quantified 1, 2 and 5 days after injection around the lesion site. Strong GSA-B4 reactivities were detected in SCI animals. The GSA-B4 expression was highest in the epicentre of lesion and appeared to be considerably weaker rostrally and caudally from lesion site. Similar changes were found in the animals received mRNA-LNP encoding eGFP treatment in all the examined time points (FIGS. 6A and 6B). Interestingly, moderately decreased GSA-B4 reactivity can be observed following intraspinal delivery of mRNA-LNP encoding hIL-10 (FIG. 6C). The quantitative analysis of GSA-B4 reactivity clearly showed that intraspinal administration of mRNA-LNP encoding hIL-10 significantly decreased the GSA-B4 reactivity compared to injured spinal cords of SCI and mRNA-GFP animals in all the examined time points (FIGS. 6D-6F). Because a significant decrease in microglia/macrophages was observed in mRNA-hIL-10 group, it was hypothesized that there is a different in the level of cytokines between injured spinal cords (SCI group) and hIL-10 treated cords (mRNA-hIL-10). Twenty-nine cytokines were evaluated with the Proteome Profiler array and displayed the tendency of cytokine changes in mRNA-hIL-10 group compared with control group (SCIgroup) (FIGS. 6G-6J). sICAM-1 and CXCL7 showed no remarkable changes in all the examined time points (FIGS. 6G and 6H). In contrast, TIMP-1 and CNTF showed trends towards increased at 2 and 5 days after hIL-10-LNP treatment (FIGS. 61 and 6J). Although protein expression of hIL-10 was significantly decreased 5 days after intraspinal administration of hIL-10 mRNA, an increasing trend in TIMP-1 and CNTF appeared as a time-delayed secondary effect. The circulating cytokines were also evaluated within the first 5 days following intraspinal injection of saline and hIL10 mRNA-LNP injection in SCI and mRNA-hIL-10 animals. CINC-1 showed only larger decreasing trend on day 2 compared with SCI group (FIG. 11A). No notable changes of other cytokine level were observed in the mRNA-hIL-10 animals compared to SCI animals (FIG. 11B). These results suggest that intraspinal administration of mRNA-LNP encoding hIL-10 has only minor systemic side effect.

### Discussion

Nucleoside-modified mRNA has numerous advantages. It does not integrate into the host genome, so requires only active translation in the cytoplasm of cells. mRNA is translated transiently in cells and is degraded by normal physiological processes in a relatively short and controllable amount of time. Due to these properties, nucleoside-modified mRNA serves as a new platform for gene therapy vector delivery in injured spinal cord.

The injured spinal cord is unable to regenerate properly due to its unfavourable environment and the inhibitory factors. Nucleoside-modified mRNA was used with a new delivery approach to express therapeutic proteins in injured spinal cord and induce neuroprotection and functional outcome. The mRNA-LNP encoding eGFP expression kinetics in intact and injured spinal cords was investigated to provide evidence for active translation of mRNA. A single injection of low dose (1.0 μg/μl eGFP mRNA-LNPs; injected 3 μg into the spinal cord) was applied into intact and injured rat spinal cords. eGFP-mRNA actively translated up to 21 days in the spinal cords. Neurons in the injured cords expressed GFP up to 14 days, whereas intact spinal cord neurons were only able to express eGFP for 5 days. Without being bound by theory, it is likely that a change in the environment caused by the injury induces this divergent expression pattern. Interestingly, microglia/macrophages were positive for eGFP for only a short time, however, astrocytes were able to express eGFP up to 21 days after intraspinal delivery of mRNA-LNPs.

Based on these results mRNA-LNP encoding hIL-10 was administrated intraspinally (1.0 μg/l hIL-10 mRNA-LNPs; injected 3 μg) 1 week after the injury. hIL-10 was used because it is responsible for ant-inflammatory effect and neuroprotection and ameliorate neuropathic pain. hIL-10-mRNA actively translated to protein in neurons, astrocytes and microglia/macrophages in injured spinal cord for up to 5 days. Similar to the eGFP, neurons, astroctyes and microglia/macrophages expressed hIL-10 within first 2 days while only neurons and astrocytes expressed hIL-10 on day 5. Although the same dose was used as in case of eGFP, IL-10 was expressed for a shorter time. This is perhaps due to the fact that IL-10 mRNA is consists of the a greater number of nucleosides and encodes a human protein, which may affect the rate and timing of expression. At the same time hIL-10 mRNA treatment induced a significant morphological and functional recovery compared to injured rats. Comparing this effect with hIL-10 delivered by an osmotic pump, a slightly better improvement was obtained following hIL-10 mRNA-LNP treatment (FIGS. 4 and 5).

Others have also successfully delivered IL-10, and functional improvements have been noticed through implantation of virus vectors, skewed the macrophage population toward an anti-inflammatory M2 macrophages (Smith, D. R., et al, 2020, Tissue Engineering Part A, 26(11-12):672-682). Delayed intraspinal administration of mRNA-LNP encoding hIL-10 resulted in decreased GSA-B4 reactivity within the first 5 days and showed a time-delayed secondary effect where increased TIMP-1 and CNTF were observed. Others have shown that increased level of circulating TIMP-1 and TIMP-3 may contribute to preservation of the blood-brain barrier (Menge, T. et al., 2012, Science Translational Medicine, 4(161):181ra150). CNTF is a neurotrophic factor that promotes remyelination by grafted or endogenous oligodendrocyte precursor cells after spinal cord injury and decrease myelin loss as well as the severity of functional loss after experimental autoimmune encephalomyelitis (Cao, Q., et al., 2010, Journal of Neuroscience, 30(8):2989-3001; Linker, R. A., et al., 2002, Nature Medicine, 8:620-624). Interestingly, only minor systemic side effect can be mentioned. Only CINC-1 showed a marked systemic decrease 2 days after hIL-10 mRNA-LNP treatment. CINC-1 is expressed by macrophages, neutrophils, and epithelial cells (Wu, X., et al., 1994, Journal of Clinical Investigations, 94(1):337-344). This cytokine is involved in the processes of angiogenesis, inflammation and wound healing (Zhang, Z-J., et al., 2013, Pain, 154(10:2185-2197).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a nucleoside-modified mRNA encapsulated in a lipid nanoparticle wherein the nucleoside-modified mRNA encodes IL10, IL-6, MIP1a, GDNF (glial cell line-derived neurotrophic factor), or a combination thereof.

2. The composition of claim 1, wherein the composition comprises a nucleoside-modified mRNA sequence encoding IL10 and a nucleoside-modified mRNA sequence encoding IL-6.

3. The composition of claim 1, wherein the composition comprises a nucleoside-modified mRNA sequence encoding IL10 and a nucleoside-modified mRNA sequence encoding MIP1a.

4. The composition of claim 1, wherein the composition comprises a nucleoside-modified mRNA sequence encoding IL10 and a nucleoside-modified mRNA sequence encoding GDNF.

5. The composition of claim 1, wherein the composition comprises nucleoside-modified mRNA sequence encoding IL10, a nucleoside-modified mRNA sequence encoding IL-6, a nucleoside-modified mRNA sequence encoding MIP1a, and a nucleoside-modified mRNA sequence encoding GDNF.

6. The composition of any of claims 1-5, wherein the nucleoside-modified mRNA comprises m1Ψ.

7. The composition of claim 1, wherein the nucleoside-modified mRNA encodes human IL10 and wherein the nucleoside-modified mRNA is encoded by the optimized DNA sequence comprising SEQ ID NO: 5.

8. The composition of claim 1, wherein the nucleoside-modified mRNA encodes for human IL10 comprising the amino acid sequence of SEQ ID NO:1.

9. The composition of claim 1, wherein the nucleoside-modified mRNA encodes for human IL6 and wherein the nucleoside-modified mRNA is encoded by the optimized DNA sequence comprising SEQ ID NO: 6.

10. The composition of claim 1, wherein the nucleoside-modified mRNA encodes for human IL6 comprising the amino acid sequence of SEQ ID NO:2.

11. The composition of claim 1, wherein the nucleoside-modified mRNA encodes for human MIP1a and wherein the nucleoside-modified mRNA is encoded by the optimized DNA sequence comprising SEQ ID NO: 7.

12. The composition of claim 1, wherein the nucleoside-modified mRNA encodes for human MIP1a comprising the amino acid sequence of SEQ ID NO:3.

13. The composition of claim 1, wherein the nucleoside-modified mRNA encodes for human GDNF and wherein the nucleoside-modified mRNA is encoded by the optimized DNA sequence comprising SEQ ID NO: 8.

14. The composition of claim 1, wherein the nucleoside-modified mRNA encodes for human GDNF comprising the amino acid sequence of SEQ ID NO:4.

15. The composition of claim 1, wherein the composition comprises:
a nucleoside-modified mRNA sequence encoding human IL10 and wherein the nucleoside-modified mRNA is encoded by the optimized DNA sequence comprising SEQ ID NO: 5;
a nucleoside-modified mRNA sequence encoding human IL-6 and wherein the nucleoside-modified mRNA is encoded by the optimized DNA sequence comprising SEQ ID NO: 6;
a nucleoside-modified mRNA sequence encoding human MIP1a and wherein the nucleoside-modified mRNA is encoded by the optimized DNA sequence comprising SEQ ID NO: 7; and
a nucleoside-modified mRNA sequence encoding human GDNF and wherein the nucleoside-modified mRNA is encoded by the optimized DNA sequence comprising SEQ ID NO: 8.

16. The composition of claim 1, wherein the composition comprises:
nucleoside-modified mRNA sequence encoding human IL10 comprising the amino acid sequence of SEQ ID NO:1;
nucleoside-modified mRNA sequence encoding human IL-6 comprising the amino acid sequence of SEQ ID NO:2;
nucleoside-modified mRNA sequence encoding human MIP1a comprising the amino acid sequence of SEQ ID NO:3; and
nucleoside-modified mRNA sequence encoding human GDNF comprising the amino acid sequence of SEQ ID NO:4.

17. A method of treating a spinal cord injury comprising intra spinally applying a composition wherein the composition comprises a nucleoside-modified mRNA encapsulated in a lipid nanoparticle wherein the nucleoside-modified mRNA encodes IL10, IL-6, MIP1a, GDNF (glial cell line-derived neurotrophic factor), or a combination thereof.

18. The method of claim 17, the composition comprises a nucleoside-modified mRNA sequence encoding IL10 and a nucleoside-modified mRNA sequence encoding IL-6.

19. The method of claim 17, wherein the composition comprises a nucleoside-modified mRNA sequence encoding IL10 and a nucleoside-modified mRNA sequence encoding MIP1a.

20. The method of claim 17, wherein the composition comprises a nucleoside-modified mRNA sequence encoding IL10 and a nucleoside-modified mRNA sequence encoding GDNF.

21. The method of claim 17, wherein the composition comprises a nucleoside-modified mRNA sequence encoding IL10, a nucleoside-modified mRNA sequence encoding IL-6, a nucleoside-modified mRNA sequence encoding MIP1a, and a nucleoside-modified mRNA sequence encoding GDNF.

22. The method of any of claims 17-21, wherein the nucleoside-modified mRNA comprises m1Ψ.

23. The method of claim 17, wherein the nucleoside-modified mRNA encodes human IL10 and wherein the nucleoside-modified mRNA is encoded by the optimized DNA sequence comprising SEQ ID NO: 5.

24. The method of claim 17, wherein the nucleoside-modified mRNA encodes for human IL10 comprising the amino acid sequence of SEQ ID NO:1.

25. The method of claim 17, wherein the nucleoside-modified mRNA encodes for human IL6 and wherein the nucleoside-modified mRNA is encoded by the optimized DNA sequence comprising SEQ ID NO: 6.

26. The method of claim 17, wherein the nucleoside-modified mRNA encodes for human IL6 comprising the amino acid sequence of SEQ ID NO:2.

27. The method of claim 17, wherein the nucleoside-modified mRNA encodes for human MIP1a and wherein the nucleoside-modified mRNA is encoded by the optimized DNA sequence comprising SEQ ID NO: 7.

28. The method of claim 17, wherein the nucleoside-modified mRNA encodes for human MIP1a comprising the amino acid sequence of SEQ ID NO:3.

29. The method of claim 17, wherein the nucleoside-modified mRNA encodes for human GDNF and wherein the nucleoside-modified mRNA is encoded by the optimized DNA sequence comprising SEQ ID NO: 8.

30. The method of claim 17, wherein the nucleoside-modified mRNA encodes for human GDNF comprising the amino acid sequence of SEQ ID NO:4.

31. The method of claim 17, wherein the composition comprises:
a nucleoside-modified mRNA sequence encoding human IL10 and wherein the nucleoside-modified mRNA is encoded by the optimized DNA sequence comprising SEQ ID NO: 5;
a nucleoside-modified mRNA sequence encoding human IL-6 and wherein the nucleoside-modified mRNA is encoded by the optimized DNA sequence comprising SEQ ID NO: 6;
a nucleoside-modified mRNA sequence encoding human MIP1a and wherein the nucleoside-modified mRNA is encoded by the optimized DNA sequence comprising SEQ ID NO: 7; and
a nucleoside-modified mRNA sequence encoding human GDNF and wherein the nucleoside-modified mRNA is encoded by the optimized DNA sequence comprising SEQ ID NO: 8.

32. The method of claim 17, wherein the composition comprises:
a nucleoside-modified mRNA sequence encoding human IL10 comprising the amino acid sequence of SEQ ID NO:1;
a nucleoside-modified mRNA sequence encoding human TL-6 comprising the amino acid sequence of SEQ ID NO:2;
a nucleoside-modified mRNA sequence encoding human MIP1a comprising the amino acid sequence of SEQ ID NO:3; and
a nucleoside-modified mRNA sequence encoding human GDNF comprising the amino acid sequence of SEQ ID NO:4.


SEQUENCE LISTING (8 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 3 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 4 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 5 - DNA, 534 bp, Homo sapiens
  source 1..534: mol_type = other DNA
atgcactcctccgccctgctgtgctgcctggtgctgctgaccggcgtgcgcgcctcccccggccagggcacccagtccgagaactcctgcacccacttccccggcaacctgcccaacatgctgcgcgacctgcgcgacgccttctcccgcgtgaagaccttcttccagatgaaggaccagctggacaacctgctgctgaaggagtccctgctggaggacttcaagggctacctgggctgccaggccctgtccgagatgatccagttctacctggaggaggtgatgccccaggccgagaaccaggaccccgacatcaaggcccacgtgaactccctgggcgagaacctgaagaccctgcgcctgcgcctgcgccgctgccaccgcttcctgccctgcgagaacaagtccaaggccgtggagcaggtgaagaacgccttcaacaagctgcaggagaagggcatctacaaggccatgtccgagttcgacatcttcatcaactacatcgaggcctacatgaccatgaagatccgcaac

SEQ ID NO: 6 - DNA, 636 bp, Homo sapiens
  source 1..636: mol_type = other DNA
atgaactccttctccacctccgccttcggccccgtggccttctccctgggcctgctgctggtgctgcccgccgccttccccgcccccgtgccccccggcgaggactccaaggacgtggccgccccccaccgccagcccctgacctcctccgagcgcatcgacaagcagatccgctacatcctggacggcatctccgccctgcgcaaggagacctgcaacaagtccaacatgtgcgagtcctccaaggaggccctggccgagaacaacctgaacctgcccaagatggccgagaaggacggctgcttccagtccggcttcaacgaggagacctgcctggtgaagatcatcaccggcctgctggagttcgaggtgtacctggagtacctgcagaaccgcttcgagtcctccgaggagcaggcccgcgccgtgcagatgtccaccaaggtgctgatccagttcctgcagaagaaggccaagaacctggacgccatcaccacccccgaccccaccaccaacgcctccctgctgaccaagctgcaggcccagaaccagtggctgcaggacatgaccacccacctgatcctgcgctccttcaaggagttcctgcagtcctccctgcgcgccctgcgccagatg

SEQ ID NO: 7 - DNA, 276 bp, Homo sapiens
  source 1..276: mol_type = other DNA
atgcaggtgtccaccgccgccctggccgtgctgctgtgcaccatggccctgtgcaaccagttctccgcctccctggccgccgacacccccaccgcctgctgcttctcctacacctcccgccagatcccccagaacttcatcgccgactacttcgagacctcctcccagtgctccaagcccggcgtgatcttcctgaccaagcgctcccgccaggtgtgcgccgacccctccgaggagtgggtgcagaagtacgtgtccgacctggagctgtccgcc

SEQ ID NO: 8 - DNA, 633 bp, Homo sapiens
  source 1..633: mol_type = other DNA
atgaagctgtgggacgtggtggccgtgtgcctggtgctgctgcacaccgcctccgccttccccctgcccgccggcaagcgcccccccgaggcccccgccgaggaccgctccctgggccgccgccgcgcccccttcgccctgtcctccgactccaacatgcccgaggactaccccgaccagttcgacgacgtgatggacttcatccaggccaccatcaagcgcctgaagcgctcccccgacaagcagatggccgtgctgccccgccgcgagcgcaaccgccaggccgccgccgccaaccccgagaactcccgcggcaagggccgccgcggccagcgcggcaagaaccgcggctgcgtgctgaccgccatccacctgaacgtgaccgacctgggcctgggctacgagaccaaggaggagctgatcttccgctactgctccggctcctgcgacgccgccgagaccacctacgacaagatcctgaagaacctgtcccgcaaccgccgcctggtgtccgacaaggtgggccaggcctgctgccgccccatcgccttcgacgacgacctgtccttcctggacgacaacctggtgtaccacatcctgcgcaagcactccgccaagcgctgcggctgcatc